(19) United States
(12) Patent Application Publication
(10) Pub. No.: US 2016/0125132 A1
(43) Pub. Date: May 5, 2016

(54) DETERMINING NUCLEIC ACID CONCENTRATION BY COUNTING NUCLEIC ACID COPIES

(71) Applicants: John SANTALUCIA, (US); Gregory J. BOGGY, (US); Eric B. Anderson, Ann Arbor, MI (US); James W. McCollum, (US); DNA SOFTWARE, INC., Ann Arbor, MI (US)

(72) Inventors: John SantaLucia, Grosse Pointe Woods, MI (US); Gregory J. Boggy, Ann Arbor, MI (US); Eric B. Anderson, Ann Arbor, MI (US); James W. McCollum, Northville, MI (US)

(21) Appl. No.: 14/435,310

(22) PCT Filed: Oct. 15, 2013
(86) PCT No.: PCT/US13/65141

§ 371 Date: Apr. 13, 2015

Related U.S. Application Data
(60) Provisional application No. 61/714,185, filed on Oct. 15, 2012.
(60) Provisional application No. 61/763,457, filed on Feb. 11, 2013.
(60) Provisional application No. 61/793,753, filed on Mar. 15, 2013.

(51) Int. Cl. G06F 19/24; C12Q 1/68

(52) U.S. Cl. CPC G06F 19/24; C12Q 1/686

(57) ABSTRACT

Provided herein is technology relating to quantifying the concentration of nucleic acids and particularly, but not exclusively, to methods for modeling data from a single quantitative amplification reaction, e.g., a PCR assay, to determine the initial concentration of a DNA target. In alternative embodiments, additional unknowns may be used, e.g., parameters for Bn, which is the concentration of probe at cycle n (and which changes in TAQMAN assays due to cleavage of the probe), EC for efficiency of probe cleavage, PDC for apparent probe dissociation constant, etc.

The present application claims priority to U.S. Provisional Application Ser. No. 61/714,185, filed Oct. 15, 2012; 61/763,457, filed Feb. 22, 2013; and 61/793,753, filed Mar. 15, 2013, each of which is incorporated herein by reference.

### FIELD OF INVENTION

Provided herein is technology relating to the determination of target DNA concentration and particularly, but not exclusively, to methods for modeling data from nucleic acid detection reactions, such as quantitative PCR assays, to determine the number of nucleic acid target molecules in a sample.

### BACKGROUND

Quantitative PCR (qPCR) is a laboratory technique for measuring the concentration of a target DNA sequence. Using quantification cycle (C_{q}) standard curve quantification of qPCR data (see, e.g., Higuchi R, Fockler C, Dollinger G, Watson R, “Kinetic PCR Analysis: Real-time Monitoring of DNA Amplification Reactions”, Bio-Technology 11: 1026-1030 (1993)), one can obtain nucleic acid concentration in a sample with high accuracy over a wide dynamic range. In this technique, a calibration curve for each amplification target is first prepared. A serial dilution of a known concentration of target nucleic acid is prepared, each diluted sample is subjected to PCR, and the accumulation of product is detected in real time, i.e., during the course of the amplification, generally by the detection of a signal (e.g., fluorescence) that accumulates in proportion to the amount of amplified material. Each sample is characterized by its C_{q}, the cycle at which the fluorescence signal increases above the quantification threshold. An unknown sample of interest (e.g., a sample suspected of containing the same target material in an unknown amount) is subjected to PCR under the same conditions, its C_{q} is measured, and the C_{q} calibration curve for that target is then used to determine the initial copy number for the unknown sample.

Another widely-used and accurate method is “digital PCR” (Vogelstein, B; Kinzler K W (1999), Proc Natl Acad Sci U S A. 96 (16): 9236-41), in which a sample is diluted to the point where, on average, a single molecule is present in each reaction in a large number of replicates, such that Poisson analysis can be used to determine the number of target molecules present in the initial sample.

The C_{q} standard curve and digital PCR methods of quantification are both time, labor, and reagent intensive. Further, the Cq standard curve method limits the through-put of qPCR, since it is performed on each qPCR plate, thereby limiting the number of wells that can be devoted to unknown samples. The C_{q} standard curve method requires that the desired target is available in pure form and in known quantities and that a series of precisely diluted standards of that target are available, which involves a complex liquid handling procedure, and in the digital PCR method, it is necessary to first determine the proper dilution to perform Poisson analysis (Simant Dube, Jian Qin, Ramesh Ramakrishnan (2008) PlosOne, 3 (8), e2876, pp. 1-9, incorporated herein by reference), and then to perform that dilution reproducibly by performing complex liquid handling.

In theory, the shape of a qPCR data curve can be used directly to quantify DNA concentration by fitting a model to qPCR data; however, current empirical model-based quantification methods are not as reliable as standard curve quantification. For example, empirical model-fitting methods, such as sigmoidal or exponential curve-fitting (see, e.g., Liu W H, Saint D A, “A new quantitative method of real time reverse transcription polymerase chain reaction assay based on simulation of polymerase chain reaction kinetics”, Analytical Biochemistry 302: 52-59 (2002); Liu W H, Saint D A, “Validation of a quantitative method for real time PCR kinetics”, Biochemical and Biophysical Research Communications 294: 347-353 (2002); Rutledge R G, “Sigmoidal curve-fitting redefines quantitative real-time PCR with the prospective of developing automated high-throughput applications”, Nucleic Acids Research 32: e178 (2004); Smith M V, Miller C R, Kohn M, Walker N J, Portier C J, “Absolute estimation of initial concentrations of amplicon in a real-time RT-PCR process”, BMC Bioinformatics 8: 409 (2007); Rutledge R G, Stewart D, “A kinetic-based sigmoidal model for the polymerase chain reaction and its application to high-capacity absolute quantitative real-time PCR”, BMC Biotechnology 8: 47 (2008)), each of which is incorporated herein by reference in its entirety, fail to quantify reliably qPCR data because they are unable to describe accurately the amplification efficiency in early cycles of qPCR, where the fluorescence signal is dominated by noise. The model-predicted behavior in these early cycles depends on assumptions about amplification efficiency implicit in the model. For example, fitting qPCR data with an exponential curve implies that amplification efficiency observed in the log-linear region of the qPCR curve is constant through all early PCR cycles, while fitting with a sigmoidal curve implies that early cycle amplification efficiency follows a sigmoidal trend. Because these assumptions are not consistent with the mechanism of PCR, empirical model predictions are less reliable than predictions made by other models.

Still other modeling methods include those presented by Namkoong, et al. in U.S. Pat. No. 8,340,919; Archer in U.S. Pat. No. 8,346,485, and the stepwise kinetic equilibrium models described by Cobbs (BMC Bioinformatics 2012 13:203), each of which is incorporated herein by reference in its entirety for all purposes.

Ideally, a qPCR assay would only require measurements on a single undiluted sample, and with a small number of replicates. As has been suggested by others, the shape of a single qPCR amplification curve should be sufficient to determine uniquely the initial DNA concentration in a sample (see, e.g., Liu (2002) (I and II); Rutledge (2004); Smith (2007); and Rutledge (2008), supra). In practice, however, the available single-assay qPCR analysis techniques have been less accurate than the gold standard technique of standard curve calibration.

### SUMMARY

Embodiments of the invention comprise methods and systems for determining the amount of a target nucleic acid, or for comparing relative amounts of target nucleic acids, comprising:
a) providing quantitative amplification data set(s) from target nucleic acid in a sample;
b) providing a mass action kinetic model of exponential amplification comprising parameters F(D₀), pdK, Pₙ, F_{b}, and Fₘ, wherein F(D₀) represents the amount of signal from double-stranded DNA at cycle 0, which is proportional to D₀, the amount of double stranded DNA at cycle 0; pdK is a constant related to the rate constant for primer hybridization divided by the rate constant determining the rate of DNA amplicon reannealing during a quantitative amplification reaction, Pₙ is the concentration of primer for cycle n; F_{b} represents a constant background fluorescence present in the data; and Fₘ represents the slope of the background fluorescence present in the data, and optionally comprising parameters Bₙ and/or EC, wherein Bₙ, is the concentration of probe at cycle n and, EC for efficiency of probe cleavage; and
c) fitting said quantitative data set with said mass action kinetic model of a quantitative amplification reaction to determine a value F(D₀) for said target nucleic acid;
e) determining a value for F′(D₀), wherein said value F′(D₀) is proportional to D₀, the amount of target nucleic acid in said sample.

In alternative embodiments, additional unknowns may be used, e.g., parameters for Bₙ, which is the concentration of probe at cycle n (and which changes in TAQMAN assays due to cleavage of the probe), EC for efficiency of probe cleavage, PDC for apparent probe dissociation constant, etc.

In certain embodiments, the quantitative amplification data set comprises data from an amplification reaction comprising a forward primer and a reverse primer, wherein the initial concentration of the forward primer, FP₀, is or is not the same as the initial concentration of the reverse primer, RP₀, and wherein said mass action kinetic model of exponential amplification comprises the parameters FP_{N} and RP_{N}, wherein FP_{N} is the concentration of forward primer for cycle n, and RP_{N} is the concentration of reverse primer for cycle n.

In some embodiments, wherein FP_{N} at cycle N is determined according to the equation:

FP_{N}=FP_{N-1}−(LS_{N}−LS_{N-1})−(ASₙ−AS_{N-1})

and RP_{N} at cycle N is calculated according to the equation:

RP_{N}=RP_{N-1}−(LA_{N}−LA_{N-1})−(AA_{N}−AA_{N-1})

In some embodiments of the inventive methods and systems, determining a value for F′(D₀) comprises determining a value for signal at an intrinsic reference point F_{reference} in said quantitative amplification data set, and in certain embodiments, the value for F′(D₀) is determined from the value of F(D₀) according to the equation:

$${F}^{′} ( {D}_{0} ) = \frac{F ( {D}_{0} )}{{F}_{reference}}$$

In some embodiments, the quantitative amplification data set of a target describes a curve, and wherein F_{reference} is selected from the group consisting of Fₘₐₓ_{_}_{curvature}, and Fₘₐₓ_{_}_{second}_{_}_{derivative}, F_{inflection}, Fₘᵢₙ_{_}_{second}_{_}_{derivative}, Fₘᵢₙ_{_}_{curvature}, and Fₘᵢₙ, wherein Fₘₐₓ_{_}_{curvature} is the fluorescence at a point of maximum curvature of said curve, Fₘₐₓ_{_}_{second}_{_}_{derivative} is the fluorescence at the point of the maximum of the second derivative F_{inflection} is the fluorescence at the inflection point in a curve of said quantitative amplification data set, Fₘᵢₙ_{_}_{second}_{_}_{derivative} is the fluorescence at the point of the minimum of the second derivative, Fₘᵢₙ_{_}_{curvature} is the fluorescence at a point of minimum curvature of said curve, and Fₘₐₓ is the fluorescence at saturation of curve, and In certain embodiments, F_{reference} is F_{inflection}.

In some embodiments, the methods further comprise a step of determining a value for D₀, wherein D₀ is proportional to the value F′(D₀) according to Equation 40:

D₀=U·F′(D₀) (40)

wherein U is a universal constant.

In some embodiments, the methods and systems, providing said quantitative amplification data set comprising exposing said one or more samples to amplification reaction reagents for quantitative amplification under conditions wherein said target nucleic acid is amplified and wherein quantitative amplification data is collected. In some embodiments, the said quantitative amplification data set is a truncated quantitative amplification data set, and in some embodiments, the truncated quantitative amplification data set is provided by exposing said one or more samples to amplification reaction reagents under conditions wherein said target nucleic acid is amplified and wherein quantitative amplification data is collected, wherein collection of quantitative amplification data is terminated at a threshold fluorescence value to generate said truncated quantitative amplification data sets.

In some embodiments, the quantitative amplification data set from said target nucleic acid comprise a plurality of replicate quantitative amplification data sets for target nucleic acid. In certain embodiments, a mean fitting error component sigma_mean_F′(D₀) is determined from the replicate quantitative amplification data set for said target nucleic acid.

In certain embodiments a median F′(D₀) is determined from at least two replicate quantitative amplification data sets for said target nucleic acids.

In some embodiments of the methods and systems of the invention, the quantitative amplification data set comprises fluorescence data, and background fluorescence parameters comprise background fluorescence constant F_{b}, and background fluorescence slope Fₘ.

In certain preferred embodiments, the quantitative amplification reaction is a polymerase chain reaction, and said quantitative amplification data set is a quantitative PCR data set.

In some embodiments of the methods and systems of the invention, the mass action kinetic model of quantitative amplification comprises the relation:

$${D}_{n} = {D}_{n - 1} + k ln ( 1 + \frac{{D}_{n - 1}}{k} ) ,$$

wherein Dₙ represents an amount of DNA in a sample at the end of an nth cycle of a quantitative amplification reaction, and k is a constant. In certain embodiments, fluorescence from DNA at cycle n F′(Dₙ) is calculated from fluorescence observed during the nth cycle of said polymerase chain reaction (Fₙ), and wherein Fₙ=F′(Dₙ)+Fₘ*n+F_{b}.

In certain embodiments of the methods and systems of the invention, a value for U is determined using a process comprising:
i) providing a target nucleic acid in a quantitative amplification reaction mixture of volume V, wherein a single copy of said target nucleic acid is present in said mixture;
ii) exposing said mixture to conditions under which said target nucleic acid is amplified to produce a quantitative amplification data set,
iii) determining values for F(D₀=1 copy) and F_{reference} from said quantitative amplification dataset;
iv) calculating a value for F′(D₀=1 copy), wherein

$${F}^{′} ( {D}_{0} ) = \frac{F ( {D}_{0} )}{{F}_{reference}}$$

v) calculating U, wherein

$${D}_{reference} = U \times V = \frac{1}{{F}^{′} ( {D}_{1 copy} )} \qquad ( 32 )$$

and
vi) optionally, computing a fraction of delayed onset.

In certain preferred embodiments, a DNA copy number for said target nucleic acid at cycle 0 is calculated according to Equation 33:

D₀=U×V×F′(D₀) (33)

In some embodiments, the methods and systems comprise determining the presence or absence of delayed onset of amplification of one or more target strands.

In some embodiments, the providing step comprises providing a plurality of replicate mixtures, each comprising a zero, one or a plurality of copies of said target nucleic acid in a quantitative amplification reaction mixture, wherein said exposing comprises exposing said plurality of replicate mixture of volume V to said conditions such that a plurality of replicate quantitative amplification data sets is produced; and wherein said determining values for F(D₀=1 copy) comprises averaging one or more data points from said plurality of replicate quantitative amplification data sets, and F_{reference} is one of the six intrinsic reference points.

In certain embodiments, the invention provides a system comprising a microprocessor configured to perform any of the methods provided by the present invention. For example, in certain embodiments, the system:
i) fits quantitative amplification data sets with a mass action kinetic model of exponential amplification comprising parameters F(D₀), pdK, Pₙ, F_{b}, and Fₘ, wherein F(D₀) represents the amount of signal from single or double-stranded DNA at cycle 0, which is proportional to D₀, the amount of double stranded DNA at cycle 0; pdK is a constant related to the rate constant for primer hybridization divided by the rate constant for amplicon reannealing of sense and antisense strands; Pₙ is the concentration of primer for cycle n; F_{b} represents a constant background fluorescence present in the data; and Fₘ represents the slope of the background fluorescence present in the data, and optionally comprising parameters Bₙ, PDC, and/or EC, wherein Bₙ, is the concentration of probe at cycle n, PDC is the apparent probe dissociation constant, and, EC is the efficiency of probe cleavage; and
ii) calculates a value for F′(D₀), wherein said value F′(D₀) is proportional to D₀, the amount of target nucleic acid in said sample.

In some embodiments, said microprocessor is further configured to determine a value for signal at an intrinsic reference point F_{reference} in said quantitative amplification data set, and wherein the value for F′(D₀) is determined from the value of F(D₀) according to the equation:

$${F}^{′} ( {D}_{0} ) = \frac{F ( {D}_{0} )}{{F}_{reference}}$$

In preferred embodiments, said quantitative amplification data set describes a curve, and wherein F_{reference} is selected from the group consisting of Fₘₐₓ_{_}_{curvature}, and Fₘₐₓ_{_}_{second}_{_}_{derivative}, F_{inflection}, Fₘᵢₙ_{_}_{second}_{_}_{derivative}, Fₘᵢₙ_{_}_{curvature}, and Fₘₐₓ, wherein Fₘₐₓ_{_}_{curvature} is the fluorescence at a point of maximum curvature of said curve, Fₘₐₓ_{_}_{second}_{_}_{derivative} is the fluorescence at the point of the maximum of the second derivative, F_{inflection} is the fluorescence at the inflection point in a curve of said quantitative amplification data set, Fₘᵢₙ_{_}_{second}_{_}_{derivative} is the fluorescence at the point of the minimum of the second derivative, Fₘᵢₙ_{_}_{curvature} is the fluorescence at a point of minimum curvature of said curve, and Fₘₐₓ is the fluorescence at saturation of curve.

In some embodiments, said microprocessor is further configured to calculate a value for D₀, wherein D₀ is proportional to the value F′(D₀) according to Equation 40:

D₀=U·F′(D₀) (40)

In preferred embodiments, said sample analysis functionality comprises a real-time PCR apparatus.

In some embodiments, the system further comprises functionality to output a result reporting a value for D₀ or a value derived from the value for D₀. In certain embodiments, said value derived from the value for D₀ comprises a concentration of target nucleic acid in a volume, or a value for a number of target nucleic acid copies in a sample. In particularly preferred embodiments, the result is understandable by a human.

The present invention also finds application in determining the relative amounts of a plurality of target nucleic acids, in a method comprising:
a) providing quantitative amplification data sets from at least two target nucleic acids in one or more samples;
b) providing a mass action kinetic model of exponential amplification comprising parameters F(D₀), pdK, Pₙ, F_{b}, and Fₘ, wherein F(D₀) represents the amount of signal from double-stranded DNA at cycle 0, which is proportional to D₀, the amount of double stranded DNA at cycle 0; pdK is a constant related to the rate constant for primer hybridization divided by the rate constant determining the rate of DNA amplicon reannealing during a quantitative amplification reaction; P₀ is the concentration of primer for cycle n; F_{b} represents a constant background fluorescence present in the data; and Fₘ represents the slope of the background fluorescence present in the data and optionally comprising parameters Bₙ and/or EC, wherein Bₙ, is the concentration of probe at cycle n and, EC for efficiency of probe cleavage;
c) fitting each of said quantitative data sets with said mass action kinetic model of a quantitative amplification reaction to determine a value F(D₀^{A}) for a target nucleic acid A, and a value F(D₀^{B}) for a target nucleic acid B;
d) calculating from F(D₀^{A}) and F(D₀^{B}) an intrinsically referenced value F(D₀^{A}) for a target nucleic acid A, and an intrinsically referenced value F′(D₀^{B}) for a target nucleic acid B, wherein said value F′(D ₒ^{A}) is proportional to D₀^{A}, the amount of target nucleic acid A, and said value F′(D₀^{B}) is proportional to D₀^{B}, the amount of target nucleic acid B in said one or more samples;
d) determining a ratio D₀^{A}/D₀^{B} to determine the relative amounts of target nucleic acid A and target nucleic acid B in said one or more samples.

In some embodiments, the quantitative amplification data sets comprise data from amplification reactions each comprising a forward primer and a reverse primer, wherein the initial concentration of the forward primer, FP₀, is or is not the same as the initial concentration of the reverse primer, RP₀, and wherein said mass action kinetic model of exponential amplification comprises the parameters FP_{N} and RP_{N}, wherein FP_{N} is the concentration of forward primer for cycle n, and RP_{N} is the concentration of reverse primer for cycle n.

In some embodiments, FP_{N} at cycle N is calculated according to the equation:

FP_{N}=FP_{N-1}−(LS_{N}−LS_{N-1})−(AS_{N}−AS_{N-1})

and RP_{N} at cycle N is calculated according to the equation:

RP_{N}=RP_{N-1}−(LA_{N}−LA_{N-1})−(AA_{N}−AA_{N-1}).

In some embodiments, the ratio D₀^{A}/D₀^{B} is determined according to the Equation 39:

$$\frac{{D}_{o}^{A}}{{D}_{o}^{B}} = \frac{U • {F}^{′} ( {D}_{o}^{A} )}{U • {F}^{′} ( {D}_{o}^{B} )} = \frac{{F}^{′} ( {D}_{o}^{A} )}{{F}^{′} ( {D}_{o}^{B} )} \qquad ( 39 )$$

In some embodiments, target nucleic acid A and target nucleic acid B have the same nucleic acid sequence, while in other embodiments, target nucleic acid A and target nucleic acid B have different nucleic acid sequences.

In some embodiments, target nucleic acid A and target nucleic acid B are in the same sample, while in other embodiments, target nucleic acid A and target nucleic acid B are from different samples.

### BRIEF DESCRIPTION OF THE DRAWINGS

These and other features, aspects, and advantages of the present technology will become better understood with regard to the following drawings:

FIG. 1 shows a shows a graphic representation of raw qPCR data (gray diamonds) with the fit (black boxes) from a qPCR copy count determination for a target having exactly one (1) copy at cycle zero.

FIG. 2 provides a flowchart representation of the MAK2 algorithm for fitting quantitative amplification data. Parameters included in the output from the MAK2 algorithm are F(D₀), k, Fₘ, F_{b}, and sigma_F(D₀).

FIG. 3 provides an exemplary plot showing an optimized fit of MAK2 (solid line) to data (points). The inset depicts the full range of data with the MAK2 fit overlaid. The large curve is a blown up view of the small box in the inset.

FIG. 4 provides a graph comparing the decrease in amplification efficiency per cycle with the increase in DNA per cycle. Amplification efficiency and normalized Dn are shown as predicted by MAK2. Note that by cycle 22 (quantification cycle, Cq), the efficiency has already dropped to 60%.

FIGS. 5A and 5B show plots of F′(D₀) vs. K (5A) and K′ (5B), for six target DNAs (GAPDH, LDHA, TFRC, CD40, HPRT, and RPLPO).

FIG. 6A shows a graph comparing the estimated relative concentrations from the MAK2 modeling to the known experimental relative concentrations of GAPDH mRNA in a series of fifteen 3-fold dilutions on GAPDH mRNA with 72 replicates of each dilution. The log-log plot shows experimental relative concentration is on the X-axis and the estimated D₀ value from MAK2 is on the Y-axis. The line at 45° in the plot represents the line of agreement between prediction and known amount. FIG. 6B shows the improved relative quantification by use of the copy counting methods discussed herein.

Note that at the nine highest concentrations, the standard error in D₀ is 10%. The standard error in the mean over 72 replicates for each of the nine highest dilutions is 0.44%. Larger errors at lower concentrations are larger due to low sampling of the Poisson distribution. The three lowest dilutions (i.e. the lowest experimental relative concentrations) have 12, 26, and 62 replicates that have D₀>0, while 60, 46, and 10 replicates have D₀=0, for the respective dilutions.

FIG. 7 shows a graph comparing the estimated D₀ to the known experimental concentrations of GAPDH mRNA in the series of dilutions analyzed in FIG. 3, in which the replicates of each dilution were averaged and using intrinsically referenced F′(D₀).

FIG. 8 shows a graph of the same GAPDH data as shown in FIG. 4, but with the inclusion of the F(D₀)=0 data points (zero target DNA) into the average. The number of replicates with F(D₀)=0 are shown above the data points for the three lowest dilutions.

FIG. 9 shows a graph showing absolute quantification by copy count determination, and applying Poisson analysis to the three lowest dilutions. The raw data for GAPDH are those given in FIG. 4 (i.e. with intrinsic referencing). The absolute quantification has a standard deviation of 11.2% due to the small number of replicates (i.e. 72 replicates) used for the Poisson Analysis.

FIG. 10 shows the graph of FIG. 6B, highlighting the data at the lowest copy count.

FIG. 11 shows an enlarged view of the data points highlighted in FIG. 10.

FIG. 12 shows an enlarged view of the data points highlighted in FIG. 10, showing the effects of delayed onset of amplification.

FIG. 13 shows a graph demonstrating absolute quantification of human heart cDNA (courtesy of Ned Sekinger, Luminex Corp.) cDNA=LDHA. Dataset is 192 replicates, run on OpenArray (Life Technologies, Carlsbad, Calif.).

FIG. 14 compares counting PCR (cPCR) to digital PCR.

FIG. 15 demonstrates the output from the copy counting technology on amplification from a single copy of a target DNA.

FIG. 16 compares features of the technology of the invention with other methods of qPCR quantitative analysis.

FIG. 17 shows examples of applications of embodiments of the technology of the invention.

FIG. 18 shows examples of instruments finding application in embodiments of the technology of the invention.

FIG. 19 shows examples of accuracy determined for embodiments of the technology of the invention.

FIG. 20 shows six intrinsic reference points on a qPCR curve. The approximate locations of the six intrinsic reference points are indicated by arrows. The x-axis is the PCR cycle number. The y-axis is the signal intensity (e.g. fluorescence). Actual PCR data are shown as boxes and the best fit asymmetric log-logistic function is shown as a smooth line.

FIG. 21 shows an example of a Chi squared P distribution from sample data from TFRC PCR (transferrin receptor protein 1), calculated as described.

FIG. 22 shows Poisson distributions for different means.

FIG. 23 shows a table showing Poisson distribution for N=384.

FIG. 24 shows a frequency analysis of TFRC data from 384 wells. Actual F1=3.23. Notice the maximums at 1.6, 3.2, and 6.4 corresponding to 0.5, 1, and 2 copies, but also note that there are other modes at 3.4, 5.2, and 7.2. This frequency graph reveals that such analysis is not reliable.

FIG. 25 shows a plot of Chi-squared P vs. mean for the TFRC data of FIG. 24. In this embodiment, the chi squared values are not monotonic or smooth, but instead are in groups, suggests an embodiment in which a search is conducted in two steps, coarse and fine.

FIG. 26 shows examples of non-Poisson distribution of data from TFRC, which has delayed onset of PCR. P>0.05 is considered valid. In this example, TFRC appears not to follow a Poisson distribution.

FIG. 27 compares copy counting error to Poisson sampling error for N=384.

FIG. 28 shows an illustration of the dynamics of qPCR for MAK2 and MAK3 models. The left panel shows the overall dynamics of qPCR. The right panel is a blow up of the inflection region. The Primer concentration (triangles) clearly decreases with PCR cycle number with a readily apparent effect well below the inflection point. The MAK2 model increases without limit, which is not physical. The “MAK3” three-parameter model (boxes) accounts for the depletion of primer with qPCR cycle number, which appears to be higher than MAK2 at low cycle numbers and then less than MAK2 in the later cycle numbers. This illustrates the distorted shape of the MAK2 curve compared to the more realistic MAK3. The fluorescence from cleavage of the probe (purple x) indicates a pattern in which the fluorescence is less than the MAK2, then greater than MAK2, and finally less than MAK2, indicating the distorted shape of the MAK2 model. The MAK2 model had the K value adjusted to make the MAK2 fit the Fluorescence curve at a Y-value of approximately 3.0E12.

FIG. 29 shows a diagram illustrating the difference between accuracy and precision.

FIG. 30 shows a graph comparing the estimated relative concentrations from the MAK2 modeling to the known experimental relative concentrations of ACTB mRNA from HL-60 in a series of 3-fold dilutions with 192 replicates of each dilution. The log-log plot shows experimental relative concentration is on the X-axis and the estimated D₀ value from MAK2 is on the Y-axis.

FIG. 31 shows a graph comparing droplet digital PCR (ddPCR) results with qPCR copy counting on serially diluted samples. Note the outlier data point for the second dilution, showing that ddPCR is unreliable for that datapoint. The copy counting results are average of 14 replicates.

FIG. 32 shows a graph comparing droplet digital PCR (ddPCR) results with qPCR copy counting, with the second dilution datapoint removed.

FIG. 33 shows a graph comparing a fit curve with original data (Bio-Rad CFX384:TaqMan assay). The number of input copies determined by ddPCR is 153,600; the number determined by qPCR copy counting is 155,774.

FIG. 34 shows a graph comparing a fit curve with original data (Roche LC480:TaqMan assay). The number of input copies determined by ddPCR is 69,200; the number determined by qPCR copy counting is 72,223.

FIG. 35 shows a graph showing detection of a single copy of a target by fitting the curve of a SYBR Green assay/LifeTech QuantStudio. The copy counting software omitted the first three cycles from the fitting.

FIG. 36 shows fitting of a curve and copy counting using data from an inefficient PCR reaction. The number of input copies determined by ddPCR is 945; the number determined by qPCR copy counting is 933.

FIG. 37 shows a graph and a table comparing expected vs. observed Poisson distribution for copy counted data. The sample is HL-60 cDNA, courtesy of Edward Sekinger, Luminex Corporation. The target is ACTB, with the assay run on a Life Technologies QuantStudio Flex 12K system.

FIGS. 38A and 38B show calibration plate results for a human EFGR assay. FIG. 38A: 36 wells of qPCR data were collected on a sample with a mean copy number of 1.543. The data were fit using the primer depletion model and the F′(D₀) values (using the inflection point as the intrinsic reference) were obtained using qPCR CopyCount. The data were then sorted (i.e. ranked) by the magnitude of F′(D₀). The graph plots the F′(D0) vs. the rank (from lowest to highest rank). The results clearly show clusters of F′(D0) values at particular values of F′(D₀). In this example, the F₁ is approximately 1.2E-12, meaning that the points with F′(D₀) within about 30% of 1.2E-12 are in fact PCR wells that had 1 molecule of target DNA at cycle zero. The data within 30% of 2.4E-12 correspond to D₀=2. The clusters at 0.6E-12 and 1.8E-12 correspond to D₀=0.5 and 1.5, respectively. FIG. 38B (N-336): The data are the same as those in Figure XA, except that the F′(D₀) values have been divided by the F₁ (i.e. the D₀ is plotted on the Y-axis). The results clearly show clusters at D₀=0.5, 1.0, 1.5, 2.0, 2.5, 3.0, etc. The half integer D₀ clusters (i.e. D₀=0.5, 1.5, 2.5, etc.) indicate that one copy of DNA has been delayed in its amplification by 1 cycle so that in fact these clusters should be rounded up to the next highest integer (e.g. D₀=0.5 should be rounded to 1, D₀=1.5 should be rounded to 2, etc.). This graph also is the basis for the “Cluster method” for determining U.

FIG. 39 shows a plot of F′(D₀) vs. rank number for human ACTB cDNA, which has little delayed onset (Delayed Onset Fraction ˜2%). F₁ in this example is approximately 1.5E-10. Note that there are few wells with D₀=0.5, 1.5, or 2.5.

### DEFINITIONS

To facilitate an understanding of the present technology, a number of terms and phrases are defined below. Additional definitions are set forth throughout the detailed description.

As used herein, “a” or “an” or “the” can mean one or more than one. For example, “a” cell can mean one cell or a plurality of cells.

As used herein, a “data point” is one datum collected during an experiment. The datum can be a numerical value (e.g., a real number, an integer, etc.) or a quality (e.g., blue, hot, off, on, long, short, etc.). Recording an observable property of an experimental system at discrete time intervals produces a time-ordered series of data points associated with a change in the properties of the experimental system as a function of time or some other characteristic that changes.

As used herein, the terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

As used herein, the term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex, which is transcribed by a cell in its natural state into a “sense mRNA.” Thus, an “antisense” sequence is a sequence having the same sequence as the non-coding strand in a DNA duplex. The term “antisense RNA” refers to a RNA transcript that is complementary to all or part of a target primary transcript or mRNA.

As used herein, “amplification” is a special case of in vitro nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, some thermostable polymerases (e.g., Taq, Pfu, etc.), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989), incorporated herein by reference in its entirety, for all purposes).

As used herein, the term “amplifiable nucleic acid” refers to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, or containing natural or artificial modified nucleotides, which is capable of acting as a point of initiation of synthesis when placed under conditions in which synthesis of a primer extension product complementary to a nucleic acid strand is induced (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The ideal lengths of the primers will depend on many factors, including the PCR annealing temperature, solution conditions (including the monovalent and divalent salts, and buffer additives such as DMSO, glycerol, formamide, TMAC, and betaine).

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195; 4,683,202; and 4,965,188 that describe a method for increasing the concentration of a segment of a target sequence in a mixture of DNA without cloning or purification. See also H. A. Erlich (ed.), PCR Technology, Stockton Press (1989). This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, typically by heating, and the primers then annealed to their complementary sequences within the target molecule, typically by cooling. Following annealing, the primers are extended with a polymerase so as to form a new pair of complementary strands. The steps of denaturation, primer annealing, and polymerase extension can be repeated many times, typically by temperature cycling (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” These amplified segments are sometimes referred to as “amplicons”.

With PCR, it is possible to amplify a single copy of a specific target sequence in DNA to a level detectable by several different methodologies (e.g., hybridization with a labeled probe; incorporation of biotinylated primers followed by avidin-enzyme conjugate detection; binding of a fluorescent dye to the amplified product; incorporation of ³²P- or fluorescently-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product”, “PCR fragment”, “amplification product”, and “amplicon” refer to the resultant polynucleotides after two or more cycles of the PCR steps of denaturation, annealing, and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, droplet, or other container with the reaction volume, etc.).

The term “real time” as used herein in reference to detection of nucleic acid amplification or signal amplification refers to the detection or measurement of the accumulation of products or signal in the reaction while the reaction is in progress, e.g., during incubation or thermal cycling. Such detection or measurement may occur continuously, or it may occur at a plurality of discrete points during the progress of the amplification reaction, or it may be a combination. For example, in a polymerase chain reaction, detection (e.g., of fluorescence) may occur continuously during all or part of thermal cycling, or it may occur transiently, at one or more points during one or more cycles. In some embodiments, real time detection of PCR is accomplished by determining a level of fluorescence at the same point (e.g., a time point in the cycle, or temperature step in the cycle) in each of a plurality of cycles, or in every cycle. Real time detection of amplification may also be referred to as detection “during” the amplification reaction.

Two types of fluorescent reporting methods are commonly used in real time detection of PCR assays. Detection can comprise use of intercalating or groove binding dyes, such as ethidium bromide, SYBR Green, SYTO, EvaGreen, etc., which bind to any double-stranded DNA generated during the PCR reaction and emit enhanced fluorescence. For homogeneous detection, intercalating or groove binding dyes are added to the PCR reagents prior to thermocycling. Although intrinsically nonspecific, DNA melt curves can be used to check melting temperature and thereby identity of the amplification products.

Another method of real time detection of amplification comprises the use of fluorogenic probes. Fluorogenic probes are generally designed to hybridize to a region of the target sequence that is between the primers, such that successful probe hybridization confirms that the intended target sequence was amplified. Such methods are advantageous in that they are less susceptible than dye-based methods to artifact signal from the amplification of primer-dimer and other incorrect amplicons. Most probe-based reporting systems use fluorescent resonance energy transfer (FRET) or static or dynamic quenching or a similar system of interactive labels as the basis of detection. In common embodiments, such as the TAQMAN and other 5′ nuclease detection assays, a 5′ nuclease cleaves the probe that has hybridized to amplification product to separate a fluorophore from a quencher molecule, thereby increasing detectable fluorescence as a reflection of the accumulation of amplicon during the amplification reaction. In some assay designs the probes are not cleaved, but change conformation in the presence of the correct target or amplicon such that the change in probe conformation separates the fluorophore from the quencher molecule (such as a molecular beacon, scorpion probe, or amplifluor probe) or separates two complementary strands with fluorophore on one strand and the other strand with a quencher (as in the bimolecular variant of scorpion probes).

As used herein, the terms “quantitative PCR” and “qPCR” are used interchangeably and refer to polymerase chain reactions conducted under conditions in which the change in the amount of amplicon can be determined during the reaction, preferably at each cycle, e.g., by real time monitoring of changes in fluorescence, such that the quantity of target material present in the reaction prior to amplification can be determined or estimated, e.g., by comparison to a reference and/or by calculation from measured fluorescence signal.

As used herein, the terms “quantitative amplification reaction” refers to an amplification reaction, e.g., polymerase chain reaction, ligase chain reaction (LCR), etc., in which the reaction is a cyclic reaction in which primer hybridization (or ligation probe hybridation) competes with re-annealing of single strands to form double-stranded DNA (as in LCR and PCR).

The term “amplification plot” as used in reference to a thermal cycling amplification reaction refers to the plot of signal that is indicative of amplification, e.g., fluorescence signal versus cycle number. When used in reference to a non-thermal cycling amplification method, an amplification plot generally refers to a plot of the accumulation of signal as a function of time.

The term “baseline” as used in reference to an amplification plot refers to the detected signal, e.g., fluorescence coming from assembled amplification reactions at prior to incubation or, in the case of PCR, in the initial cycles, in which there is little signal from the amplicon DNA.

The term “Cₜ” or “threshold cycle” and “C_{q}” or “quantification cycle” have the same meaning, and as used herein in reference to real time detection during an amplification reaction that is thermal cycled refers to the fractional cycle number at which the detected signal (e.g., fluorescence) passes a defined threshold.

As used herein, the term “reverse-transcriptase PCR” or “RT-PCR” refers to a type of PCR where the starting material is mRNA. The starting mRNA is enzymatically converted to complementary DNA or “cDNA” using a reverse transcriptase enzyme. The cDNA is then used as a template for a PCR reaction.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), flash media, and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “sample” is used in its broadest sense. For example, in some embodiments, it includes a specimen or culture (e.g., microbiological culture), whereas in other embodiments, it is meant to include both biological and environmental samples (e.g., suspected of comprising a target sequence, gene or template). In some embodiments, a sample may include a specimen of synthetic origin. Samples may be unpurified or may be partially or completely purified or otherwise processed.

The present invention is not limited by the type of biological sample used or analyzed. The present invention is useful with a variety of biological samples including, but are not limited to, tissue (e.g., heart, lung, tumor or other biopsy tissue), cells (e.g., blood cells, muscle cells, tumor cells, etc.), gas, bodily fluid (e.g., blood or portion thereof, serum, plasma, urine, semen, saliva, etc), or solid (e.g., stool) samples obtained from a human (e.g., adult, infant, or embryo) or animal (e.g., cattle, poultry, mouse, rat, dog, pig, cat, horse, and the like). In some embodiments, biological samples may be plants, food, and/or feed products and/or ingredients such as dairy items, vegetables, meat and meat by-products, grain products, and/or agricultural waste. Biological samples may be obtained from all of the various families of domestic animals, as well as feral or wild animals.

Environmental samples include but are not limited to soil, water (e.g., freshwater or seawater), algae, lichens, geological samples, air containing materials containing nucleic acids, crystals, and industrial samples, as well as samples obtained from food and dairy processing instruments, apparatus, equipment, utensils, disposable and non-disposable items.

As used herein, the term “quantitative amplification data set” refers to the data obtained during quantitative amplification of the target sample, e.g., target DNA. In the case of quantitative PCR, the quantitative amplification data set is a collection of fluorescence values obtained during amplification, e.g., during a plurality of, or all of the PCR cycles. Data for quantitative PCR is not limited to data collected at any particular point in a PCR cycle, and fluorescence may be measured at a discrete point in each cycle or continuously throughout each cycle.

As used herein, the term “truncating” refers to the process of removing data points from a quantitative amplification data set, e.g., for the purpose of providing a set of data points for fitting by the MAK2, primer depletion, probe depletion or other models described herein. A quantitative amplification data set is truncated to include some or all of the informative data points (e.g., data points that follow the exponential-type trend of PCR), and to exclude some or all data points that do not follow the exponential-type trend of PCR (e.g., excessively noisy data in early cycles, outlier data points in the middle of the PCR, or data in the saturation phase of PCR (e.g., late cycles). In some embodiments, truncating comprises truncating a quantitative amplification data set to a cycle n either above or below the inflection point, but usually below the curvature minimum point of the qPCR curve.

As used herein, the term “truncated quantitative amplification data set” refers to the data set that results from truncating the quantitative amplification data set. The truncated quantitative amplification data set is the set of data points that is fitted with the MAK2 model or the primer or probe depletion models. A truncated quantitative amplification data set may be produced, e.g., by removing data points from a collected quantitative amplification data set, or by ceasing collection of data from a quantitative amplification reaction, e.g., by stopping the reaction or turning off a detector.

As used herein, the term “mass action kinetic model” refers to a model derived from the mass action kinetics of a reaction. Such models involve the use of ordinary differential equations to model the changes in concentration of reaction species over the course of a reaction.

As used herein, the phrase “mass action kinetic model of exponential amplification comprising parameters F(D₀)and k” refers to the MAK2 mass action kinetic model of PCR, in which the parameter F(D₀) represents the fluorescence associated with D₀, the target DNA concentration at cycle 0 of PCR, and the parameter k represents the characteristic PCR constant that determines the rate of DNA accumulation during PCR. Some embodiments of mass action kinetic modeling comprise different or additional parameters. For example, as used herein, the MAK3 model comprises the MAK2 parameter and includes primer depletion as a third parameter. In other embodiments a mass action kinetic model includes the MAK2 parameters and additional includes probe depletion as a parameter, or both primer and probe depletion parameters.

As used herein, the term “fitting” refers to the process of optimizing the fit of a mathematical function, such as the MAK2 model and the models that include primer and/or probe depletion, to a set of data. Fitting implies an iterative process, in which parameter values are defined and an objective function (e.g., the sum of squares of residuals) is evaluated. The ultimate goal of fitting is to minimize the value of the objective function by identifying the optimal set of parameter values.

As used herein, the term “fitting said truncated quantitative amplification data set with said mass action kinetic model” refers to fitting the truncated quantitative amplification data set with the MAK2 model of PCR in order to find the optimal parameter values for F(D0) and k. These optimal parameter values describe the behavior of the reaction from which the fitted data were obtained.

As used herein, the term “proportional to the amount of target nucleic acid in said sample” indicates that the value of the parameter that is referred to is correlated with the amount of target nucleic acid in the sample that is being analyzed by MAK2-fitting.

As used herein, the term “threshold fluorescence value” refers to a fluorescence value that, if crossed by the quantitative amplification data, results in an action being taken. For example, a threshold fluorescence value could be defined such that if fluorescence values in the quantitative amplification data increase to a level above this threshold, the quantitative PCR reaction is terminated, or above which data is analyzed or is no longer collected.

As used herein, the term “Nelder-Mead optimization” refers to the process of using the downhill simplex optimization method described by Nelder and Mead (see, e.g., Nelder J A & Mead R. “A simplex method for function minimization”. Computer Journal 7: 308-313 (1965)) to find the optimal fit of a mathematical model to a set of data.

As used herein, the term “Levenberg-Marquardt optimization” refers to use of the Levenberg-Marquardt nonlinear optimization algorithm (see, e.g., Press, W. H., Teukolsky, S. A., Vetterling, W. T., and Flannery, B. P. (1996) Numerical Recipes in C, 2nd Edition, Cambridge U. Press, Cambridge, pp. 683-688) to find the optimal fit of a mathematical model to a set of data. The Levenberg-Marquardt optimization algorithm is the preferred optimization algorithm for finding the least sum of squares for fitting of nonlinear models to data.

### DESCRIPTION OF THE INVENTION

The Description of the Invention comprises the following sections:
I. General description of the invention
II. Mechanistic modeling with MAK2
   II.a. Two parameter model
   II.b Primer depletion model
   II.c Asymmetric primer depletion model
   II.d Probe depletion model
III. Improved F(D₀) determination using Intrinsic Referencing
   III.a. Inflection point
   III.b Max curvature
   III.c Max second derivative
   III.d Max
   III.e Minimum second derivative
   III.f Minimum curvature
IV. Calibration of U
   IV.a. Empirical calibration of U
   IV.b. General calibration of U
   IV.c. Theoretical calibration of U
   IV.d calibration plate methods
V. Counting copies of DNA
   V.a. Fitting the qPCR curve
VI. Experimental Examples
   VI.a Exemplary fitting using MAK2
   VI.b qPCR Copy Counting
   VI.c Two-step Calibration Procedure for determining U
   VI.d Calculation of Copy Counting Calibration Error and Poisson Error
   VI.e Initial estimation of parameters for probe deletion model

### I. GENERAL DESCRIPTION OF THE INVENTION

Embodiments of the invention are described in here and in the Summary, above, which is incorporated here by reference. Although the disclosure herein refers to certain illustrated embodiments, it is to be understood that these embodiments are presented by way of example and not by way of limitation. U.S. Patent Application Ser. No. 61/714,185, filed 15 Oct. 2012, and 61/763,457, filed 22 Feb. 2013 are incorporated herein by reference in their entireties for all purposes. Although polymerase chain reaction is discussed as an exemplary amplification system that is amenable to quantification by copy counting, it is to be understood that the copy counting process can be used in the analysis of any amplification assay system in which a signal (e.g., fluorescence signal), accumulates in an exponential-like or sigmoidal manner as a function of cycle number. The technologies of the invention may be applied to the analysis, for example, of ligase chain reactions, or any cyclical reaction in which primer hybridization competes with re-annealing of single strands to form double-stranded DNA (as in LCR and PCR).

The present invention provides methods of determining absolute concentration of nucleic acids in samples analyzed, e.g., by quantitative amplification. Consider a simple analogy of counting the number of apples in a basket: this can be accomplished by weighing all the apples in the basket, subtracting the weight of the basket, and dividing by the resulting weight of the collection of apples by the weight of one apple. Similarly, the number of copies of a DNA molecule may be counted by measuring the fluorescence of all the copies of the DNA molecule, e.g., in a reaction, subtracting background fluorescence, and dividing the fluorescence of the collection of DNA copies by the fluorescence of a single copy of the DNA.

In many qPCR applications, analysis of a sample is directed to determining the amount of a target nucleic acid as compared to another sample, or the amounts of multiple different nucleic acids within a single sample. Analysis of the proportions of nucleic acids of interest is generally referred to as “relative quantification” of target nucleic acids. While highly useful, relative quantification has limitations compared to absolute quantification. Absolute quantification provides the actual number of DNA copies in the original sample (i.e., D₀). As such, absolute quantification is simpler to interpret biologically than relative quantification. Absolute quantification is essential for some applications, such as determination of viral titer, bacterial load, cancer load, siRNA dosing, or fragment library quantification for next generation sequencing. In addition, many relative quantification methods (such as the delta-delta-C_{T} method) are dependent upon reproducing the exact experimental protocol on different samples, and are thus limited to analyses of same target nucleic acid in different samples. These limitations prevent the use of relative quantification for meta analyses of archived qPCR data sets collected using different assays, on different instruments, with different reagents, collected by different groups at different times, and/or across different targets.

The present technology provides methods of performing a one-time assay calibration, such that absolute quantification can be determined for each and every PCR reaction with precision of <10% (depending upon the user pipetting error, sample preparation errors, etc.) or the Poisson limit (i.e. the square root of the number of copies), whichever is larger. With a small number of replicates (e.g. 16) the error in the absolute copy number of an unknown sample can be reduced to below 1% if suitable calibration has been performed. The method works on different samples, using different fluorophores, different solution conditions, and/or different instruments, e.g. archived qPCR data sets can also be converted using the methods of the present technology to determine absolute copy numbers, thereby increasing the value of existing data sets and enabling meta-analysis studies that were not previously possible.

Absolute quantification of nucleic acids finds use in any application in which relative quantification may be used. Quantification of nucleic acids in amplification reactions finds use in, e.g., analysis of copy number (e.g., copy number variation or “CNV”) of a target gene, gene expression profiling, non-coding RNA quantitation (e.g.,ribosomal RNAs microRNA, siRNA, snRNA, riboswitches, catalytic RNA, etc.), measuring allele frequency in a population, determining percent methylation, screening for aneuploidy and polyploidy (e.g., in prenatal screening or other genetic testing of a locus or gene), SNP genotyping, disease resistance screening, marker-assisted selection, measurement of tumor burden, zygosity screening, etc. See, e.g., Stankiewicz and Lupski, Structural Variation in the Human Genome and its Role in Disease, Annual Review of Medicine Vol. 61: 437-455 (2010), incorporated herein by reference in its entirety for all purposes.

Quantification of target nucleic acid finds particular use in copy number determination and CNV analysis, in which the signals from different target nucleic acid sequences are compared, e.g., to each other, or to control sequences of known quantity (e.g., single copy genes, housekeeping mRNAs). Analysis of copy number is useful, for example, to detect copy number variations due to, e.g., gene amplification, aneuploidy, polyploidy, cell transformation with transgenes, loss of heterozygosity, etc.

Quantification finds use in comparing the amounts of nucleic acids in samples regardless of the source of the samples. For example, in some embodiments, relative quantification is applied to analysis of the number of copies of nucleic acids within a single cell or cell type (e.g., analysis of chromosome numbers in fetal cells in prenatal testing, zygosity testing, single nucleotide polymorphism and allele testing, relative gene expression, etc.) In other embodiments, relative quantitation is applied to the analysis of nucleic acids from different target sources, e.g., in determining relative populations of different organisms in a sample (e.g., different microbes in a fecal or soil sample; different plant strains in a mixture of plant-derived sample matter, e.g., for grain or seed testing and grading).

Numerous agricultural applications are also found, e.g., in quantification of alleles, genes, and/or transgenes in plants and animals, and for determining absence or presence, the gene dosage, zygosity, etc., of any nucleic acid of interest.

### Intrinsic Referencing

One of the problems with working with qPCR data is that each locus in an instrument at which a qPCR reaction is conducted (e.g., each well of a qPCR plate; each tube in a rotor or block; each reaction site on a chip or other surface (e.g., in an array), each aqueous droplet in an emulsion etc.) is its own “instrument” in that each locus has its own illumination intensity, detection geometry, and materials in the optical path. In the past, one method for dealing with variation between reaction loci was to include a passive dye reference, such as ROX dye, in each reaction, and to normalize across a plate to that value. However, such ROX normalization introduces its own error, including disadvantageously correcting for sample volume. In addition, use of ROX correction still does not allow for corrections to be applied across different machines. ROX also has its own fluorescence excitation and emission spectra, which can limit the fluorophores that can be used in singleplex or multiplexed qPCR reactions.

The combined technologies of intrinsic referencing and counting PCR (cPCR) have advantages over other quantitative PCR methods, such as digital PCR, delta-delta-C_{T} method and C_{T} standard curve method, and quantification by UV absorbance. The combination of cPCR with intrinsic referencing, as discussed in more detail below, results in a method that requires no internal or external standards, no special instrumentation (beyond a standard qPCR instrument), is machine independent, requires no dilution series, and can be performed on either a single qPCR reaction, or on replicates that are averaged to reduce the error. The method described herein is also superior in error and robustness compared to alternative curve fitting methods such as: LINREG, method of Rutledge, and others.

Absolute quantification provides the number of copies of DNA in a sample and is simpler to interpret biologically than relative quantification, which provides an indirect quantification (like the C_{T}, or delta-delta-C_{T}, or a percentage change compared to some standard). The cPCR method and qPCR CopyCount and other methods (e.g., qPCR AUTOFIT, (DNA Software, Inc.) have numerous applications including but not limited to: viral titer, NGS fragment library quantification, fetal trisomy diagnostics, determination of copy number variation, rare allele detection, and meta-analysis of archived data sets.

Embodiments of the present invention comprise methods of normalizing fluorescence data. During development of the technology, a method termed “intrinsic referencing,” in which the fluorescence data from qPCR are self-normalized to a particular point on the qPCR curve, was developed. Such intrinsic referencing reduces the standard deviation in relative quantification by more than 3 fold. This improvement in relative quantification precision also finds application in the methods described hereinabove of quantifying the DNA targets by counting copies, termed “counting PCR” or cPCR. Intrinsic referencing removes the machine- and fluorophore-dependence of the measurement.

One aspect of intrinsic referencing comprises finding a point in a qPCR curve that corresponds to a particular number of DNA target copies, regardless of the starting number of copies of target DNA. The number of copies of target DNA at that point does not need to be known, but whatever the number is, a practitioner of the method first finds that point. There are at least six embodiments discovered during the course of developing the technology that are suitable for intrinsic referencing: i) maximum of the curvature; ii) maximum second derivative; iii) inflection point (i.e. the point where second derivative is zero); iv) minimum second derivative; v) minimum of the curvature; and vi) maximum saturation level. In certain preferred embodiments, intrinsic referencing comprises normalizing to the inflection point. Generally, the inflection point method is preferred because it provides the smallest standard deviation.

It is contemplated that, in some embodiments, any multiple of cycle number of the inflection point (or multiple of one of the other reference points discussed above) or multiple of the fluorescence corresponding to the inflection point cycle also finds use in the methods of the present invention. It is further contemplated that any multiple of the six reference points may also be used. It is still further contemplated that the reference point or points need not be limited to an integer, such as a cycle number. For example, in certain embodiments, it is a preferred practice to perform a numerical or analytical optimization procedure to find the fractional cycle point corresponding to the reference.

Methods of normalizing to the inflection point are not limited to any particular embodiment. It is contemplated that there are a number of different ways of determining the inflection point that find application in the technology. In some embodiments, the inflection point is determined by, e.g., numerical fitting of a polynomial to the raw data, then performing analytical determination of inflection point by solving for the point where the second derivative=0. In other embodiments, the point is determined by performing numerical interpolation of the raw data. In certain preferred embodiments, the inflection point is determined by performing best fit Log-logistic function to the raw data, then performing analytical determination of inflection point by solving for the point where the second derivative=0.

The technology is not limited to particular ways of applying the intrinsic referencing to each qPCR curve. For example, in some embodiments, intrinsic referencing is applied to all of the data in the curve, prior to fitting the curve to the model, e.g., the MAK2 model. In certain preferred embodiments, the data are fitted prior to application of the intrinsic referencing.

In some embodiments, data from technical replicates are averaged. It is contemplated that there are numerous way of aggregating the technical replicates of qPCR data into an average that find application in the technology. In some embodiments, outlier data are removed, the remaining data are averaged, and the data is then fitted to the model. In some preferred embodiments, each well or locus is fitted, the outliers are then removed, and the data from the replicates are then averaged.

Some embodiments of the technology comprise calibrating a proportionality constant U, e.g., for new assay designs for counting PCR. In some embodiments, assay calibration is done on a theoretical basis, e.g., the calibration factor is computed from first principles, based on the composition of the amplification reaction. In other embodiments, the calibration is a general approximation based on assumptions about typical reaction composition (e.g., concentration of primers, concentration of probes, typical amplicon length, concentrations of dNTPs, Mg⁺⁺, primers, primer length, presence of minor groove binder (MGB) in the probe, double or single stranded status of the original target nucleic acid, the reaction PdK, etc.). In certain preferred embodiments, the calibration is an empirical assay calibration, e.g., comprising: diluting a sample so as to provide an average of approximately one target copy per well (or reaction locus); performing qPCR (or other quantitative amplification detection) to produce a data set, deducing an average F′(D₀) for one copy of target nucleic acid, and using that value for cPCR analysis of samples having unknown numbers of target nucleic acid copies. In some embodiments, the empirical calibration comprises a two-step procedure comprising initial calibration, followed by amplification and analysis of unknowns.

In some embodiments, the technologies of the present invention comprise fluorescence detection, and are not limited to the manner in which fluorescence is produced in a reaction, or to any particular dye or fluorophore. In some embodiments, the quantitative amplification data sets are derived from probe-based detection such as, “TaqMan,” 5′ nuclease assays, Molecular Beacon probes, scorpion probes, Amplifluor, and the like. Probes may be labeled with any fluorophore, including but not limited to FAM, VIC, JOE, HEX, TexasRed, TET, TAMRA, Cy5, Cy3, Cy3.5, Cy5.5, Cy7, TMR, ROX, NED, PET, ALEXA_350, ALEXA_405, ALEXA_430, ALEXA_488, ALEXA_532, ALEXA_546, ALEXA_555, ALEXA_568, ALEXA_594, ALEXA_633, ALEXA_647, ALEXA_660, ALEXA_680, ALEXA_700, and ALEXA_750.

In some embodiments, intercalator and/or groove-binding dyes may be used to provide fluorescence indicative of amplification. Such dyes include but are not limited to SYBR Green, SYTO-13, Eva Green, MGB, ethidium bromide, and the like.

It is contemplated that detectable signals other than fluorescence also find use in the methods and technologies of the invention. For example, in the detection of radioisotopes, the values for “F” in the calculations and equations provided herein represent detected units of radiation in place of detected units of fluorescence. The technology is applicable to forms of detection and/or measurement including but not limited to electrical conductivity, pH, radioactivity, densitometry, electrochemical, photon absorbance, mass spectrometry, surface plasmon resonance, chemical reactivity, zero mode waveguide, and various labeling technologies including electron spin labels, and neutron absorption label.

Application of the technologies of the invention to analysis of quantitative amplification datasets is not limited to any particular system configuration. For example, in some embodiments, the technologies of the present invention are provided to users for installation on an individual user computer (e.g., provided as an installable file). In other embodiments, the technology is provided as a component integrated with a detection instruments, e.g., a qPCR instrument or other machine. In yet other embodiments, the technologies are provided as a private enterprise installation, e.g., on a server, in a computing facility, or in another configuration of computational resources internal to an enterprise. In yet other embodiments, the technology is provided as an accessible resource, e.g., on a virtual private network, via a web site, or as a cloud-based computation resource, e.g., for public or secure access via the internet using, for example, a corporate computer, personal computer, tablet PC, cell phone or other device. In still other embodiments, the technology is provided to users as a service, e.g., for processing data sets submitted to a central location, wherein output information (e.g., target nucleic acid copy number or concentration, value of F(D₀=one copy), universal constant for the users' instrument, etc.) is returned to the user. In some embodiments, an exemplary workflow is as follows:
A user collects the needed qPCR data and uploads the data to the qPCR cloud-based service;
The service carries out the analysis using the technologies disclosed of the invention, including, e.g., the following:
   Determining the range of data points that are to be analyzed;
   Determining which qPCR wells have a true sample versus noise;
   Carrying out replicate averaging, outlier detection, and statistical analysis, including error analysis;
Results, e.g., absolute DNA copy numbers, are provided to the user, for example, in a .txt or .csv file that can be opened in a spreadsheet (e.g., MS EXCEL) or in a text editor. In some embodiments, a separate analysis file may also be provided, containing the details of the replicate averaging, outlier detection, and/or statistical analysis that were carried out.

In some embodiments, the technologies of the present invention are applied to the product of multiplexed amplification reactions, e.g., in which different amplicons produce signals using different fluorescent dyes. In some embodiments, amplicons amplified in a single reaction are compared to each other, e.g., to measure relative quantification, whereas in some embodiments, different amplicons from a single reaction are quantified without reference to each other.

It is contemplated that the technologies of the invention find application in the improvement of digital PCR (see, e.g., Vogelstein, B; Kinzler K W (1999), Proc Natl Acad Sci U S A. 96 (16): 9236-41). For example, in some embodiments, the counting PCR methods may be applied to digital PCR datasets to improve determination of wells (or other reaction loci) that have data (i.e., those that contained a copy of target nucleic acid) from the loci that do not. In some embodiments, the number of copies of DNA in each well may be determined (e.g., 0, 1, >1) e.g., so that statistics converge faster in the data analysis. Still further, in some embodiments the methods find application in providing an initial determination of sample concentration, such that the number of dilutions necessary for testing is reduced to achieve an optimal concentration range for digital PCR (e.g., so that wells have approximately 0.5 to 3.5 target copies, on average).

### II. MECHANISTIC MODELING WITH MAK2

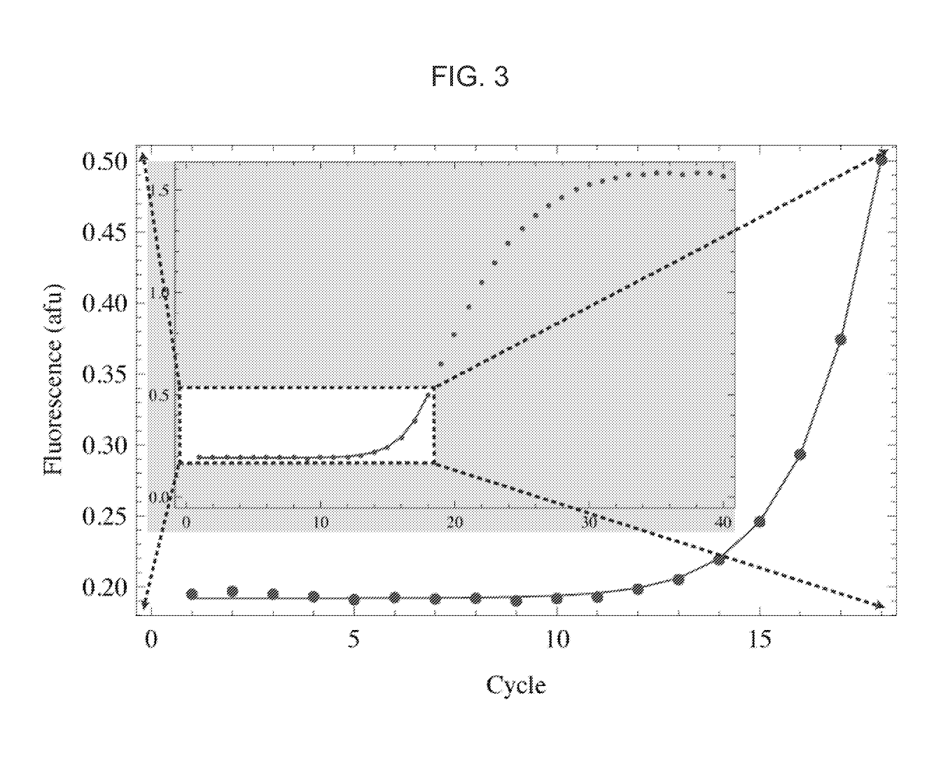

One component of the present technology is use of the mechanistic modeling of qPCR, e.g., the MAK2 modeling of Boggy and Woolf (Boggy G J and Woolf P J, PLoS One 5(8) e12355 (2010), which is incorporated by reference herein in its entirety, including all Supporting Information published therewith, including electronic files Text S1; Figure S1; Table S1; Data set S1; Data set S2; Data set S3 and Data set S4, found at doi:10.1371/journal.pone.0012355.s007; U.S. Patent Appl. Ser. Nos. 61/528,758, filed Aug. 29, 2011 and Ser. No. 13/598,599, filed Aug. 29, 2012, both incorporated herein by reference in their entireties). Unlike other fitting methods, the MAK2 uses a model that has been derived from PCR kinetics to fit qPCR data (i.e., a mechanistic model). The MAK2 fitting used in the present invention has been shown to produce results that have equivalent quality (i.e., with low error) to Cq standard curve quantification, without the need for producing a standard curve for calibration. Further, because the MAK2 model is grounded in the mechanism of PCR, there is theoretical justification for truncating data or reaction before the “plateau phase” of PCR (as diagrammed, e.g., in FIG. 3).

Experiments performed to compare the accuracy of MAK2-fitting to other qPCR quantification methods, including applying quantification methods to qPCR dilution series data indicate that quantification by MAK2-fitting is as reliable as standard curve quantification for a variety of DNA targets and a wide range of concentrations. As such, MAK2 quantification provides improved technologies for designing and analyzing qPCR experiments. For example, MAK2 provides accurate quantification for assays in which construction of a standard curve is impractical, such as portable qPCR assays with limited sample throughput (Boggy and Woolf, supra).

### II.a Two Parameter Model

MAK2 describes the accumulation of amplicon DNA during PCR. The model is derived from the reaction kinetics of the annealing and elongation steps of PCR. MAK2 is expressed as Equation 1:

$${D}_{n} = {D}_{n - 1} + k ln ( 1 + \frac{{D}_{n - 1}}{k} ) \qquad ( 1 )$$

where Dₙ represents the amount of double-stranded DNA (dsDNA) after n cycles of PCR. In Equation 1, Dₙ is recursively dependent on Dₙ₋₁, the amount of D from the previous cycle. F(Dₙ), the fluorescence associated with dsDNA at cycle n, is related to Dₙ by the relation, Dₙ=F(Dₙ)*c, where F(Dₙ) is related to overall fluorescence at cycle n by the relation, Fₙ=F(Dₙ)+Fₘ*n+F_{b}. Fₘ and F_{b} are the slope and intercept of a linear model of the background fluorescence (Boggy and Woolf, supra)

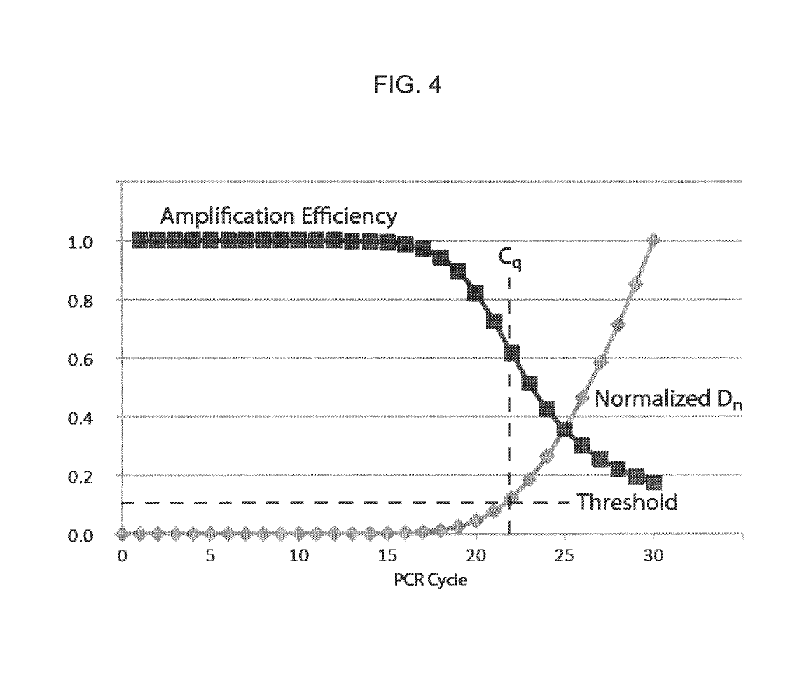

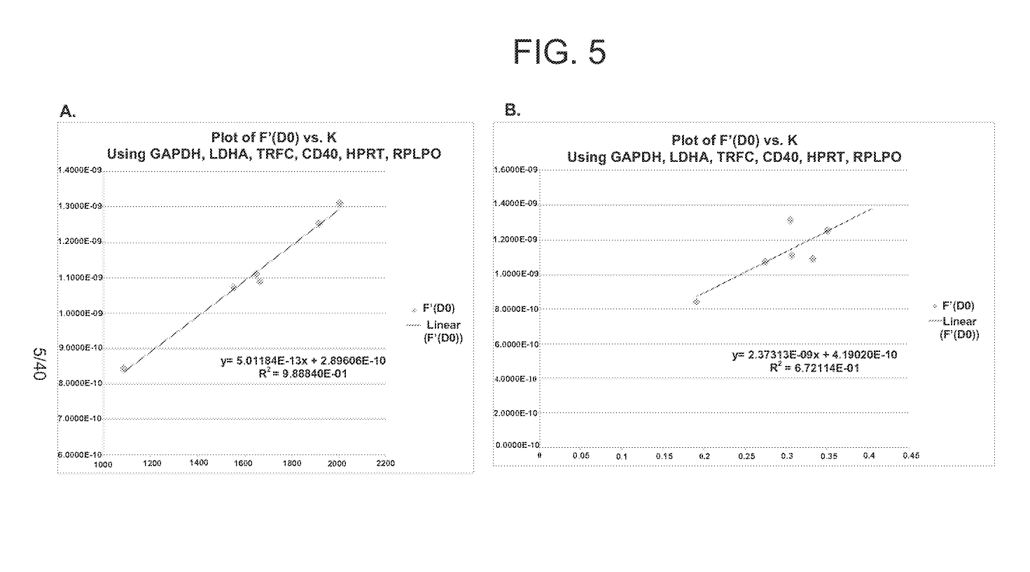

The two parameters in MAK2, D₀ and k, are sufficient to describe complex PCR behavior accurately for early cycles of qPCR, where effects such as primer depletion or polymerase saturation can be neglected. The initial target DNA concentration, D₀, determines the point at which the fluorescence signal rises above noise. The parameter k represents the ratio of primer binding and DNA reannealing rate constants and assumes a fixed primer concentration (i.e. pseudo first order amplicon reannealing rate constant) and dictates how amplification efficiency changes at every cycle with increasing DNA concentration. FIG. 4 illustrates that amplification efficiency is not constant, but that it declines steadily during PCR. Note that amplification efficiency has already declined significantly at the quantification cycle. The constant amplification efficiency value obtained by the Cq standard curve method (Rutledge and Cote, “Mathematics of quantitative kinetic PCR and the application of standard curves”, Nucl. Acids Res., 31:16 (2003), incorporated herein by reference) is actually an “effective amplification efficiency” that incorporates the effects of declining amplification efficiency on a cycle-by-cycle basis. Amplification efficiency on a cycle-by-cycle basis according to the MAK2 model is calculated as described by Boggy and Woolf, supra. As such, the characteristic PCR constant k determines the rate of DNA accumulation during PCR.

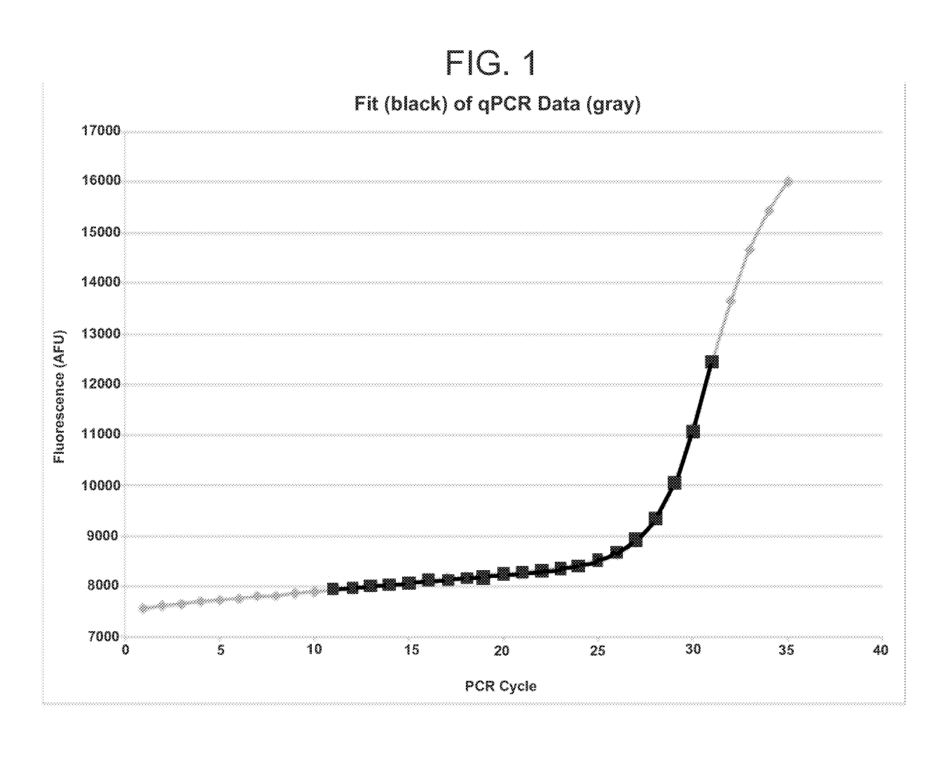

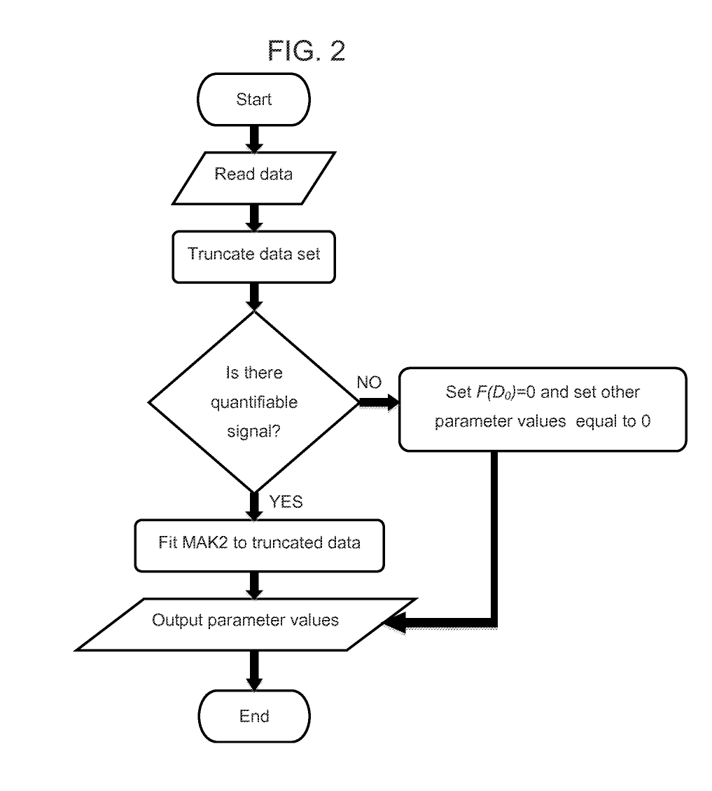

D₀ and k are the only two adjustable parameters that determine Dₙ values at every PCR cycle. These parameters have distinct effects on the shape of the MAK2 curve; changing the value of D₀ shifts the curve right or left, while changing the value of k changes the slope of the curve, as shown in FIG. 1. Fitting qPCR data to a MAK2 curve provides an estimate of fluorescence due to DNA at cycle 0 (F(D₀)). When one knows the conversion factor correlating an amount of fluorescence to an amount of DNA, the DNA concentration at cycle 0 can be inferred.

The MAK2 model and its variants that correct for background fluorescence fit the shape of the qPCR curve to determine with high accuracy the fluorescence from DNA at cycle zero, F(D₀), which is proportional to the absolute number of copies of DNA in the original sample, called D₀, according to the following equation:

D₀=c·F(D₀) (2)

where c is a proportionality constant. For absolute quantification, a value for c must be determined, e.g., by an independent calibration for each qPCR assay design using either the Cq standard curve method or digital PCR, or using the copy counting PCR analysis discussed below. There is often background fluorescence in qPCR data that is independent of the signal associated with accumulation of the amplified target. This background fluorescence is due to fluorescence produced by the reaction system itself (e.g., due to imperfect quenching of the fluorophore of the unhybridized probe molecules) or from fluorescence caused by plastics or other reagents or from cleavage of the fluorophore from the probe by mechanisms that do not involve an exonuclease activity (such as spontaneous cleavage due to hydrolysis). In model-fitting approaches for quantifying qPCR data, the fluorescence is typically assumed to be composed of the signal from DNA and linear background fluorescence. Similarly, for MAK2-fitting of qPCR data, fluorescence is background-adjusted by the parameters F_{b} and Fₘ as follows (Equation 3):

Fₙ=F(Dₙ)+Fₘ·n+F_{b} (3)

where F_{b} represents the constant background fluorescence intercept, Fₘ represents the slope of the background fluorescence and F(Dₙ) is the MAK2-predicted fluorescence due to dsDNA at cycle n. Fₙ is the variable used for fitting qPCR fluorescence data.

In some embodiments, when the MAK2 fitting is done, the qPCR curve is truncated to select the most informative region, and in some preferred embodiments, e.g., in excessively noisy qPCR curves, outlying data (“outliers”) may be filtered out prior to curve fitting. In still further embodiments, qPCR curves are statistically analyzed to provide improved estimates of error in F(D₀). In preferred embodiments, to improve fitting of the MAK2 model to the raw quantitative amplification data, the number of data points prior to the most informative region of the data should be limited so that patterns occurring in the background do not adversely affect the fit of the data and the resulting parameter values. In some embodiments, a certain number of data points prior to the most informative region of data are included in the fit (e.g., 20 data points before the inflection point), so that enough background data is present to provide good estimates of parameters Fₘ and F_{b}, but not so much background data that the fit of the data is compromised. In other embodiments, a method for identifying corrupting background signal can be employed (e.g., by measuring trends in the data) in order to find the best data point to truncate the amplification data set prior to the most informative region of the data.

One of the features of the MAK2 model embodiment is that it assumes the primer concentration is fixed and is not depleted with each cycle of PCR. In reality, however, the primer concentration is finite and is depleted during the PCR and this depletion is one cause of the saturation of PCR. The omission of primer concentration as a factor in the model leads to a shape of the qPCR curve that is not precisely modeled by MAK2, even with adjustment of k. Further, for TaqMan probes (labeled probes digested by the polymerase in each cycle of amplification), the amounts of both primers and probes are depleted with each cycle of PCR. Note that for molecular beacons and duplex dyes (e.g. SYBR green), the probe concentration is constant and unchanging. For the case where the probe is provided at a lower concentration than the primers, which is the usual case, the probe concentration depletes earlier than the primers and such probe depletion is a major cause of reaction saturation. Other factors include consumption of NTPs (which is amplicon-length dependent), enzyme deactivation, and enzyme inhibition, e.g. by pyrophosphate byproducts of amplification. Inclusion of the primer and probe depletion effects may result in better fitting (i.e. smaller fitting residuals) and also less variance in the U parameter for different assays, and less dependence on the number of data points used in the beginning part of the qPCR curve. Thus, in some embodiments, depletion of reaction components such as primer and probe is included in the modeling. Inclusion of primer and probe depletion allows for the fitting of more of the qPCR curve than the MAK2 model, which requires truncation of the data below the inflection point. In contrast, the primer and probe depletion models in certain preferred embodiments truncates the data to include the points from a beginning point (somewhere between cycle 1 and the several cycles below the curvature maximum) up to an ending point (typically above the inflection point and below the minimum second derivative).

MAK2 works from an assumption that the k parameter is the ratio of the pseudo-first-order rate constant for primer hybridization (i.e. assuming the primer concentration is fixed) over the rate constant for amplicon reannealing. This assumption is posited because in the early cycles of PCR the primer is in vast excess and thus the forward rate constant can be approximated as a pseudo-first order rate constant. This is equivalent to assuming that the [primer] does not change with PCR cycle number, which is not correct for later cycles of qPCR. Thus, MAK2 does not apply well near the inflection point and beyond. However, even well before the inflection point, the primer depletion may affect the shape of the qPCR curve. Thus, in some embodiments it is preferred to use a different k, e.g., a k^{new}, alternatively called the “primer depletion K”, pdK, as the ratio of the reaction rate constants (i.e. not the pseudo first order rate constant that is used in k), with pdK separated from the primer concentration. As a result, the observed k is directly proportional to the primer concentration. This was experimentally verified (i.e. doubling the primer concentration doubled the k value in MAK2, but pdK in the primer depletion model does not change upon doubling the primer concentration).

### II.b Primer Depletion Model

Inclusion of Primer Depletion into MAK2:

We will consider primer depletion initially from the perspective of molecule counts, and then additionally from the perspective of measuring and modeling fluorescence.

First, we convert the initial primer concentration into the number of molecules of primer:

P₀=V×N_{A}×[primer] (Eqn. 4)

Where [primer] is given by the user, V is the volume in liters, and N_{A} is Avogadro's number 6.02214E23. P₀ is the number of molecules of Primer at cycle zero. The amount of primer at cycle N, P_{N}, is given by the amount in the previous cycle minus the amount that is extended by the polymerase into an amplicon molecule in cycle N, which is given by D_{N}, −D_{N-1}, as shown below:

P_{N}=P_{N-1}−(D_{N}−D_{N-1}) (Eqn. 5)

P_{N} is the number of primer molecules that remain at cycle N. Next, we include the pdK and the cycle dependent primer concentration into the MAK2 model to give:

$${D}_{n} = {D}_{n - 1} + pdK \times {P}_{N - 1} \times LN ( 1 + \frac{{D}_{N - 1}}{pdK \times {P}_{N - 1}} ) \qquad ( Eqn . 6 )$$

The number of unknowns in this primer depletion variant of MAK2 is 2: D₀ and pdK. The number of molecules of primer at cycle zero, P₀, can be provided by the user. In certain preferred embodiments, the P₀ can be an additional unknown fitted parameter. This recursive equation converts primer molecules into DNA amplicon molecules, thereby modeling the actual process that occurs in PCR catalyzed by the polymerase enzyme. The total DNA in each cycle is the original amount at cycle zero plus the amount from primer extension.

### II.c Asymmetric Primer Depletion Model

In some embodiments, it is useful to account for the separate buildup of sense and antisense strands, and/or account for probe depletion, e.g., in TaqMan 5′ nuclease assays. The discussion above pertains to a single “primer concentration”, e.g., FP₀=RP₀=P₀ computed from Eqn. 4. In some embodiments, separate values for primer concentration may be set, to account for asymmetric PCR.

FP₀=V×N_{A}×[forward primer] (Eqn. 4B)

RP₀=V×N_{A}×[reverse primer] (Eqn. 4C)

where FP₀ and RP₀ are the number of molecules of forward and reverse primers at cycle zero, respectively. In discussing the individual strands of amplification product, the following definitions and abbreviations are used:
GS=Genomic Sense strand DNA=constant=D₀
GA=Genomic Antisense strand DNA=constant=D₀
LS=Linear Sense strand DNA
LA=Linear Antisense strand DNA
AS=Amplicon Sense strand DNA
AA=Amplicon Antisense strand DNA
S_{N}=GS+LS+AS=total Sense strand DNA at cycle N
A_{N}=GA+LA+AA=total Antisense strand DNA at cycle N
Delayed_onset=input from the calibration plate. If a calibration plate is not available, then good defaults are 0.2 for double stranded genomic DNA and 0.1 for single stranded DNA (e.g. cDNA). Limits: 0<=Delayed_onset<1
“Linear” DNA refers to products from extension of primers on the original target DNA, which accumulate at a linear rate.

The following model accounts for the possibility of asymmetric PCR and the effect of delayed onset, e.g., due to behavior of long target DNAs that may influence the efficiency of DNA copying in initial cycles (e.g., due to the incomplete denaturation of long or coiled DNA and strand reannealing). A simple modification of equation 6 allows prediction of the number of copies of amplicon sense strands, AS_{N}, and amplicon antisense strands, AA_{N}, as follows (implementing an assumption that the pdK parameter is the same for both FP and RP and for Linear and Amplicon targets):

$$\begin{matrix}{LS}_{N} = {LS}_{N - 1} + ( 1 - Delayed\_Onset ) \times pdK \times {FP}_{N - 1} \times LN ( 1 + \frac{{GA}_{0}}{pdK \times {FP}_{N - 1}} ) & ( Eqn . 6 B ) \\ {LA}_{N} = {LA}_{N - 1} + ( 1 - Delayed\_Onset ) \times pdK \times {RP}_{N - 1} \times LN ( 1 + \frac{{GS}_{0}}{pdK \times {RP}_{N - 1}} ) & ( Eqn . 6 C ) \\ {AS}_{N} = {AS}_{N - 1} + pdK \times {FP}_{N - 1} \times LN ( 1 + \frac{{LA}_{N - 1} + {AA}_{N - 1}}{pdK \times {FP}_{N - 1}} ) & ( Eqn . 6 D ) \\ {AA}_{N} = {AA}_{N - 1} + pdK \times {RP}_{N - 1} \times LN ( 1 + \frac{{LS}_{N - 1} + {AS}_{N - 1}}{pdK \times {RP}_{N - 1}} ) & ( Eqn . 6 E )\end{matrix}$$

where FP_{N-1} and RP_{N-1} are the number of molecules of forward primer and reverse primer at cycle N−1. Note the A_{N-1} in the S_{N} eqn. 6B and the S_{N-1} in the A_{N} eqn. 6C . In eqns. 6B and 6C the S₀ and A₀ are unknowns. If the target is known to be double stranded (i.e. DS=true), then S₀=A₀=D₀. If the target is single stranded (i.e. DS=false), then GS₀=D₀ and GA₀=0 (or conversely GS₀=0 and GA₀=D₀).

Delayed onset is accounted for by noting that it only applies to the formation of linear amplicons. This assumption is valid because it is the GS-GA duplex that can re-anneal and cause delayed onset by preventing the primers from binding. The reannealing of linear and exponential amplicons is already accounted for in the pdK parameter and the primer depletion with each cycle of PCR. Note that this method for accounting for delayed onset (in equations 6B and 6C) is sufficient when the number of initial copies of genomic DNA (i.e. GS₀ and GA₀) is greater than 5. If these values are less than 5, the experimental result would be stochastic, such that delayed onset would be observed sometimes and not observed at other times. Nonetheless, the implementation given here gives correct average behavior even for small D₀ values.

After LS_{N}, LA_{N}, AS_{N} and AA_{N} are computed, FP_{N} and RP_{N}, (the count of Forward Primer and Reverse Primer at cycle N) are computed, using a generalization of Equation 5

FP_{N}=FP_{N-1}−(LS_{N}−LS_{N-1})−(AS_{N}−AS_{N-1}) (Eqn. 5B)

RP_{N}=RP_{N-1}−(LA_{N}−LA_{N-1})−(AA_{N}−AA_{N-1}) (Eqn. 5C)

### Primer Depletion in Fluorescence Space:

An alternative embodiment to the count-based primer depletion model is to fit in Fluorescence space. The idea here is to express all countable quantities, such as copies of DNA and copies of primer, as their equivalent amount of fluorescence. Such an approach is applicable to the primer depletion model, but not to the probe depletion model (described below), where there is no simple way to convert DNA and primers into equivalent fluorescence quantities.

Equation 6 is converted into fluorescence by realizing that the fluorescence from DNA is proportional to the number of molecules of DNA.

F(D_{N})=D_{N}*(reference fluorescence−background)/(U*V) (Eqn. 7)

where F(D_{N}) is the fluorescence from DNA at cycle N, U is the universal constant for the given assay as described below in section III.a, V is the volume in liters, D_{N} is the number of copies of DNA at qPCR cycle N, background=N*Fₘ+F_{b}, and the reference fluorescence is the value of the fluorescence at the intrinsic reference point (e.g. the inflection point or the max second derivative, or any of the other intrinsic reference points described in section III). Equation 7 is a rearrangement of Equation 33 in section III.a. Equation 7 is converted into total fluorescence by adding in the slope and intercept of the background fluorescence.

Fₜₒₜₐₗ=F_{DNA}(D_{N})+N*Fₘ+F_{b} (Eqn. 8)

For the sake of fitting a curve directly to fluorescence values, the primer depletion relationships can be converted to and modeled in terms of fluorescence values. This is done by converting the primer count to an estimate of the potential fluorescence that would be contributed by extending the primers. First, the relationship between the initial primer count and the initial primer concentration is considered.

P₀=primerConc*N_{A}*V (Eqn. 9)

where P₀ is the initial primer count (i.e. the number of primer molecules at cycle zero), primerConc is the molar concentration of primer and N_{A} is Avogadro's number 6.022E23. Applying the conversion to fluorescence (cf. Eqn 7) yields the initial potential fluorescence (i.e. the fluorescence that would be added by extending all of the initial primers).

Fₚₒₜₑₙₜᵢₐₗ(P₀)=primerConc*N_{A}*(reference fluorescence−background)/U (Eqn. 10)

Eqn 10 sets an upper limit on the fluorescence that could be produced (excluding background), which can be combined with K and the MAK2 equation as follows.

F_{upperbound}=F_{DNA}(D₀)+Fₚₒₜₑₙₜᵢₐₗ(P₀) (Eqn. 11)

Kᵢ=K*Fₚₒₜₑₙₜᵢₐₗ(Pᵢ)=K*(F_{upper bound}−F_{DNA}(Dᵢ)) (Eqn. 12)

F_{DNA}(D_{N})=F_{DNA}(D_{N-1})+K_{N-1}*LN(1+F_{DNA}(D_{N-1})/K_{N-1}) (Eqn. 13)

As before, equation 8 is used to convert the fluorescence from DNA in Equation 13 into total fluorescence at cycle N by adding in the slope and intercept of the background fluorescence.

F_{Total}=N*Fₘ+F_{b}+F_{DNA}(D_{N-1})+K_{N-1}*LN(1+F_{DNA}(D_{N-1})/K_{N-1}) (Eqn. 14)

The inclusion of primer depletion into MAK2 results in a better fit of qPCR curves. MAK2 with Primer depletion is the preferred embodiment for fitting qPCR reactions detected by dye binding (e.g. SYBR, SYTO, EvaGreen). The primer depletion model is also applicable to TaqMan probes, though TaqMan is better modeled by the primer and probe depletion model described below.

### II.d Probe Depletion Model

When we added primer depletion to MAK2, that consideration provided a limitation on the additional fluorescence according to the quantity of primers available for extension. However, that analysis implicitly assumes that the extended primers would produce fluorescence. Probe depletion considers the conversion of probe molecules (e.g. TaqMan probes) into cleaved fluorophore molecules. As the PCR progresses to higher cycle numbers, both the primer and probe concentrations are depleted and thus the shape of the qPCR curve for a TaqMan assay reflect such depletion and thus we describe a new model that accounts for depletion of both primers and probes.

First, we convert the initial probe concentration into the number of molecules of probe:

B₀=V×N_{A}×[probe] (Eqn. 15)

Where [probe] is given by the user, V is the volume in liters, and N_{A} is Avogadro's number 6.02214E23. B₀ is the number of molecules of Probe at cycle zero. Second, the TaqMan probe is cleaved, resulting in a decrease in probe concentration as given by:

B_{N}=B_{N-1}−[BD]_{N}×V×N_{A}×CL (Eqn. 16)

B_{N} is the number of probe molecules that remain at cycle N, and [BD]_{N} is the amount bound of probe to amplicon DNA at cycle N, but before cleavage occurs. CL is the cleavage efficiency (the initial guess is CL=1, but that is allowed to float between 0 to 1 during the fit). For a given cycle, the amount bound is given by the solution of the binding equation, which involves the solution to the quadratic equation (see appendix for derivation).

ax²+bx+c=0 (Eqn. 17)

where x=[BD]. The proper numerical solution to the quadratic equation is described in Numerical Recipes (pp. 183-184). For our case, b is always negative, and the physical (i.e. positive) root is given by:

$$x = \frac{- b - \sqrt{{b}^{2} - 4 ac}}{2 a} or x = \frac{2 c}{- b + \sqrt{{b}^{2} - 4 ac}} \qquad ( Eqns . 18 )$$

where the equation on the right has superior numerical convergence for the case where c is small compared to a, which it usually is. The a_{N}, b_{N}, and c_{N} coefficients from equation 17 for each cycle N in the PCR are given by:

$$\begin{matrix}{a}_{N} = 1 & ( Eqn . 19 ) \\ {b}_{N} = \frac{- ( {D}_{N} - {D}_{N - 1} ) - {B}_{N - 1}}{V \times {N}_{A}} - PDC & ( Eqn . 20 ) \\ {c}_{N} = \frac{( {D}_{N} - {D}_{N - 1} ) \times {B}_{N - 1}}{{( V \times {N}_{A} )}^{2}} & ( Eqn . 21 )\end{matrix}$$

Where PDC is the probe Dissociation constant. PDC=1/Keq, where Keq is an effective equilibrium binding constant. Note that for N=cycle zero, the D_{N-1} in Eqns. 20 and 21 are set to zero (since by definition there is nothing before cycle zero). PDC is a new unknown (so now the model involves three unknowns and thus is called “MAK3”, Mass Action Kinetics model with 3 unknowns) that controls the steepness of the upper bend in the qPCR curve. (A large value results in a very sharp bend and a small value results in a weak bend.) An initial estimate for PDC=1.0E-8, which corresponds to a medium sharpness upper bend. Physically, K_{eq}=1/PDC is an apparent association equilibrium constant for probe binding to the template and is given by:

$${K}_{eq} = {e}^{\frac{- Δ {G}_{T}^{o}}{RT}} \qquad ( Eqn . 22 )$$

Note that in reality, equilibrium probe binding is not typically achieved since sometimes the primer gets fully extended either before the probe binds or (less likely) the probe falls off before the primer extends sufficiently to allow the exonuclease activity. Thus, the K_{eq} parameter is really an apparent equilibrium constant that accounts for the amount of probe binding that occurs in the time before the primer is fully extended. Below the inflection point, the K_{eq} can be kept fixed so that the rest of the unknowns can be solved. In certain embodiments the PDC=1/Keq can be assumed to be zero, which is equivalent to assuming that all non-annealed amplicon sense strand molecules are bound by probe molecules.

Computation of the Fluorescence:

For TaqMan, the observed fluorescence is given by the amount of fluorescence from the previous cycle, plus any probe that is cleaved in the current cycle:

F_{N}=F_{N-1}+(B_{N-1}−B_{N}) (Eqn. 23)

Derivations:

$$B + D ↔ BD$$

$${K}_{eq} = \frac{[ BD ]}{[ B ] [ D ]}$$

$${B}_{tot} = [ B ] + [ BD ]$$

$${D}_{tot} = [ D ] + [ BD ]$$

$${K}_{eq} = \frac{[ BD ]}{( {B}_{tot} - [ BD ] ) ( {D}_{tot} - [ BD ] )} = \frac{x}{( {B}_{tot} - x ) ( {D}_{tot} - x )}$$

$${K}_{eq} {B}_{tot} {D}_{tot} - {K}_{eq} x ( {B}_{tot} + {D}_{tot} ) + {K}_{eq} {x}^{2} - x = 0$$

$${x}^{2} - ( {B}_{tot} + {D}_{tot} + \frac{1}{K} ) x + {B}_{tot} {D}_{tot} = 0$$

$${ax}^{2} + bx + c = 0$$

Note that in the equations above, Bₜₒₜ and Dₜₒₜ are concentrations. Elsewhere herein these values are expressed as numbers of molecules. To covert to concentrations to numbers of molecules, the factor of V*N_{A} is used, as shown in Eqn. 15.

Note that the Dₜₒₜ in the above derivation is not the actual total DNA concentration, but instead is the amount that is not hybridized to a complementary strand (i.e. reannealed amplicon). Fortunately, we have already determined the amount of free DNA that is not hybridized, which is given as:

Dᵢₙₜ=D_{N}−D_{N-1} (Eqn. 24)

If one combined a model oriented to molecular counts, as is described above, with a factor to convert from counts to fluorescence, such a combination might be used as one embodiment to fit with the fluorescence data. On the other hand, as a different embodiment, for the sake of fitting a curve directly to fluorescence values the primer and probe depletion relationships can be converted to and modeled in terms of fluorescence values rather than counts.

The embodiment in terms of fluorescence values rather than molecular counts begins with the earlier implementation of the fluorescence oriented form of modeling primer depletion (without probe depletion). It was observed that one can convert from counts or concentrations to an equivalent potential fluorescence value, such as by using equation 10 for converting from available primer concentration to potential primer fluorescence units.

Fₚₒₜₑₙₜᵢₐₗ(P₀)=primerConc*N_{A}*(reference fluorescence−background)/U (Eqn. 10)

This same relationship, originally expressed for primers, can be used to convert from a probe concentration to a comparable potential fluorescence value for probes. As probes are consumed and converted into fluorescence, this potential fluorescence from probes is consequently depleted until it reaches zero (i.e. there are no more probes to convert from potential fluorescence to actual fluorescence).

For dyes, fluorescence was closely related to the total number of copies of DNA, so the idea of potential fluorescence from primers represented the fluorescence that would result from extending those primers. For TaqMan, even though the fluorescence comes from the cleaving of the TaqMan probes rather than from the quantity of DNA, nevertheless we retain the use of primer potential fluorescence as a way to track the increase in DNA copies and the depletion of primers. Although it is necessary to continue to track the increase of DNA and the decrease of primers just as before, for the TaqMan model with probe depletion the actual fluorescence values produced are based on probe cleavage, which can be distinct from primer extension.

One can view the calculation of fluorescence in the TaqMan model in terms of contrast with what would have been produced from a dye model. For example, at every cycle, the fluorescence units of the TaqMan model will be less than the dye model by the amount of fluorescence produced by dyes for the initial DNA at cycle zero. This is absent from the TaqMan curve because it produces fluorescence only as new DNA copies are produced and probes are cleaved, whereas dye fluorescence is related to all of the DNA copies present. In addition, as has been described above, not every primer extension results in a cleaved probe due to considerations such as probe depletion and cleavage efficiency.

Therefore, the TaqMan model with primer depletion can be implemented in terms of adding and accumulating a portion of the new fluorescence that a dye model would have produced for that cycle. Thus, a similar algorithm to the dye model can be used with the adjustment of determining what portion of each new cycle's fluorescence potential would be actualized and accumulated. This is calculated by applying the solution given above for determining what portion of the new primer extensions involve a bound probe, and also applying a factor for the portion of bound probes to successfully cleave.

### III. IMPROVED D₀ DETERMINATION USING INTRINSIC REFERENCING

“Intrinsic referencing” is a method to remove scale information from the qPCR data and to put all qPCR curves on the same scale. For example, such intrinsic referencing removes instrumental variation and fluorophore dependent effects.

Each well in a qPCR experiment can be viewed as a separate “instrument” even though the different wells are acquired on the same physical device. For different machines, the instrument variations are even larger. Such different instruments have different illumination intensity, different materials in the optical path, and different detection geometry, filtering, and detection efficiency. Different fluorophores (including both covalently attached fluorophores such as FAM and duplex binding dyes such as SYBR-Green) also change the Y-axis scale of qPCR data because different fluorophores have different absorbance extinction coefficients, different quantum yields, and different amounts of quenching (due to collisional deactivation or due to impurities in each sample). Such variations corrupt comparisons of qPCR data from different wells or from different machines. To remove such variations due to different instruments and fluorophores, qPCR CopyCount uses intrinsic referencing. The key to intrinsic referencing is to have a method that can accurately determine a particular reference point (called F_{reference}) on the qPCR curve regardless of the starting amount of DNA (or RNA) and regardless of changes that multiply all the points in the qPCR by an arbitrary constant (so called “Scale changes”) such as occurs for different instruments and fluorophores. This generally requires analysis of the shape of the qPCR curve without regard for the Y-axis scaling. Since both the F(D₀) and F_{reference} are acquired with the same instrument and with the same fluorophore, the resulting ratio of those two quantities will be instrument and fluorophore independent.

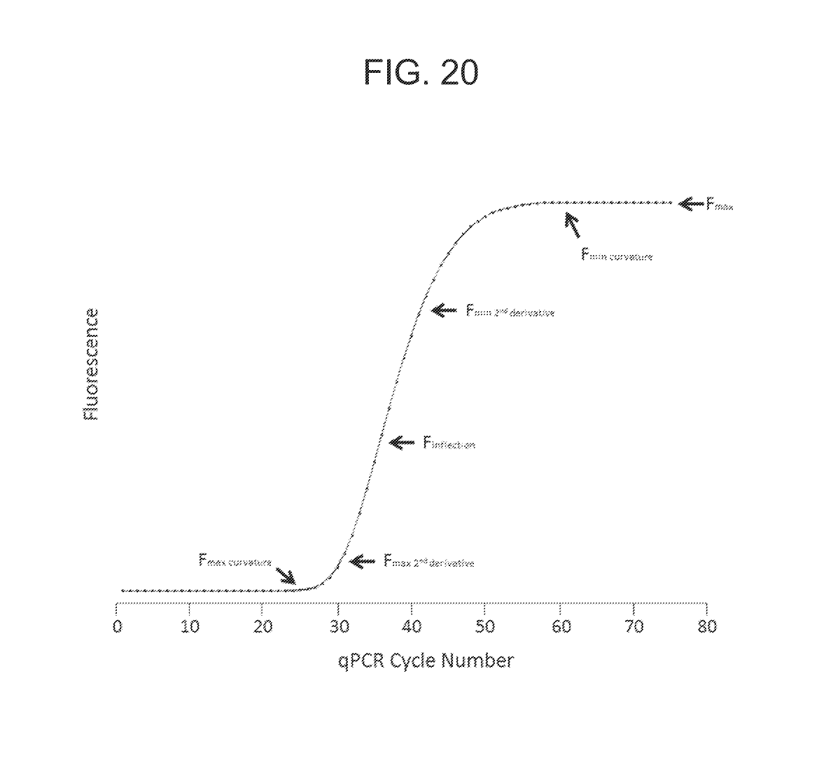

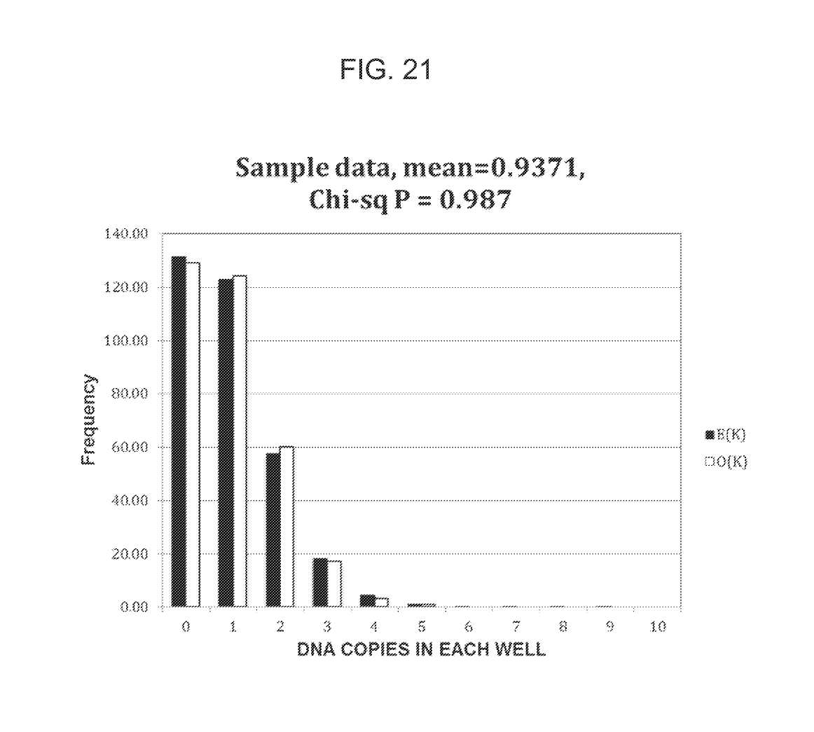

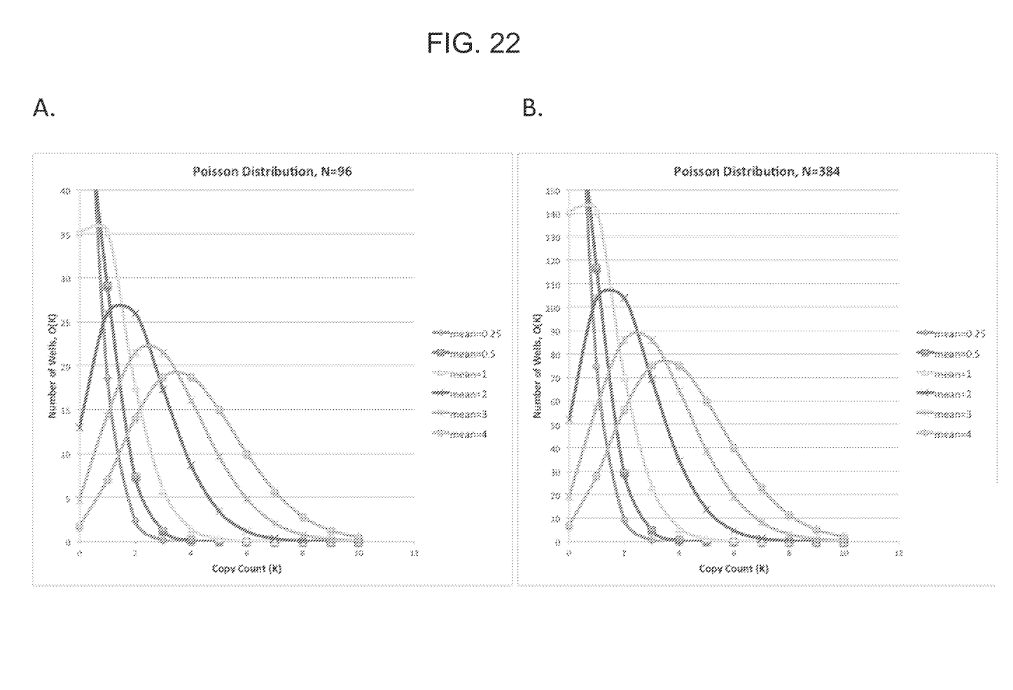

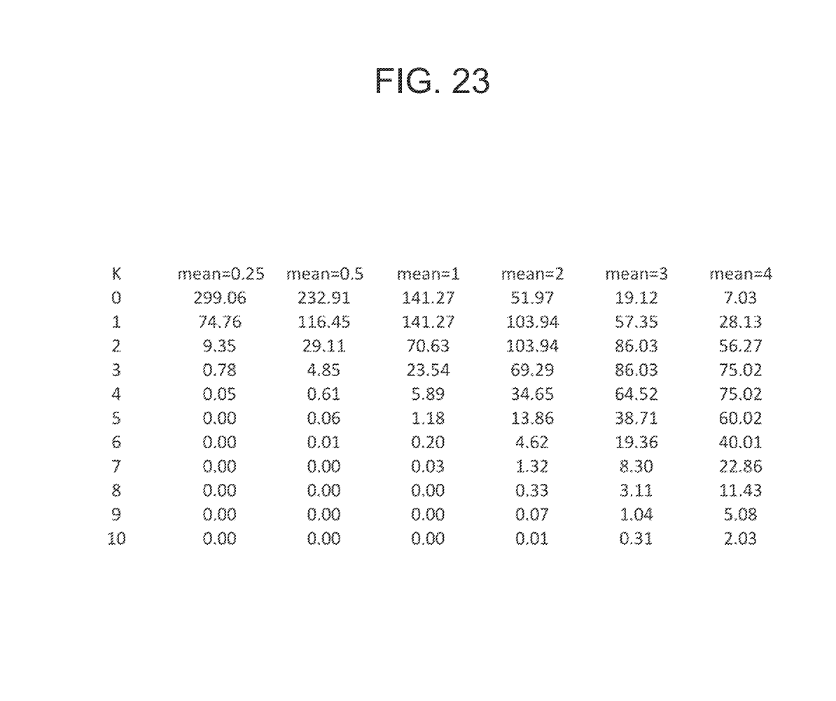

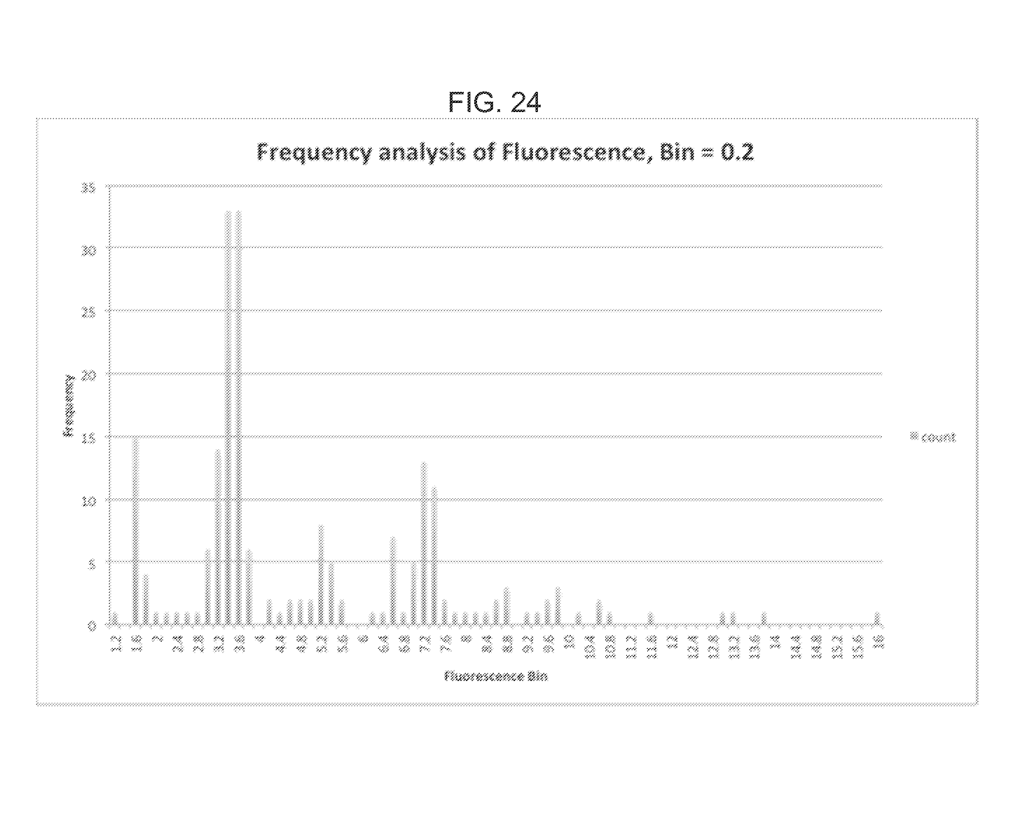

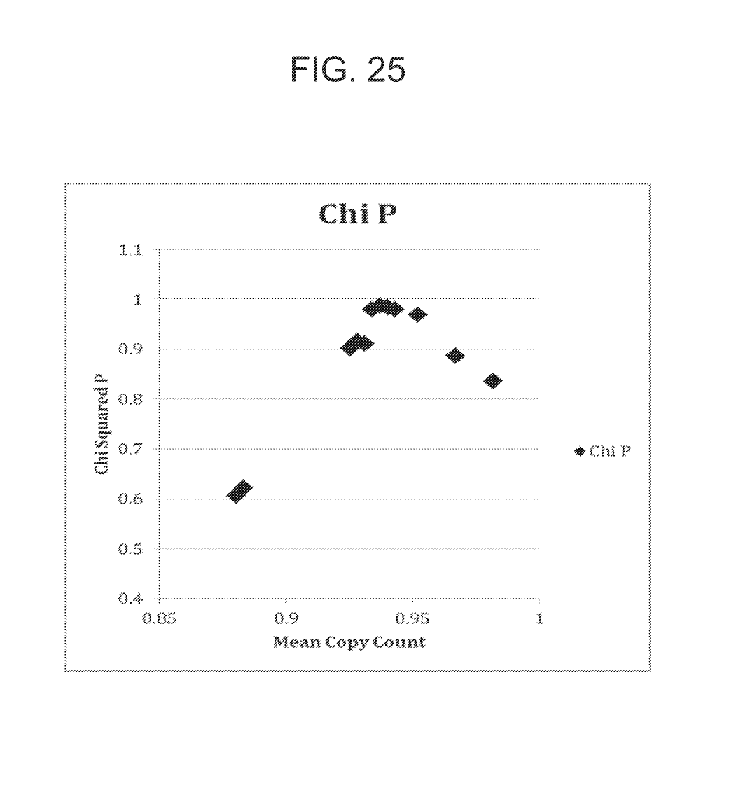

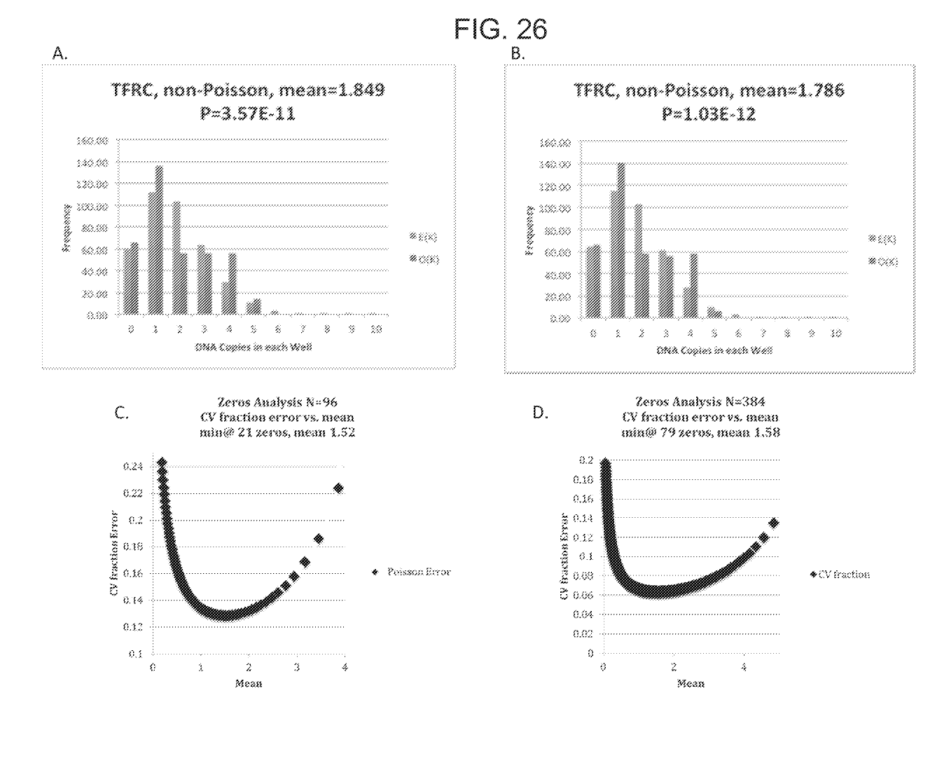

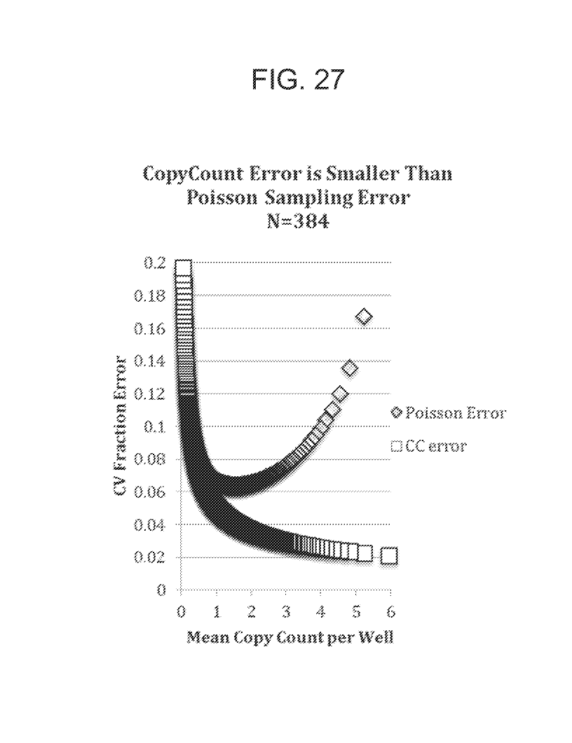

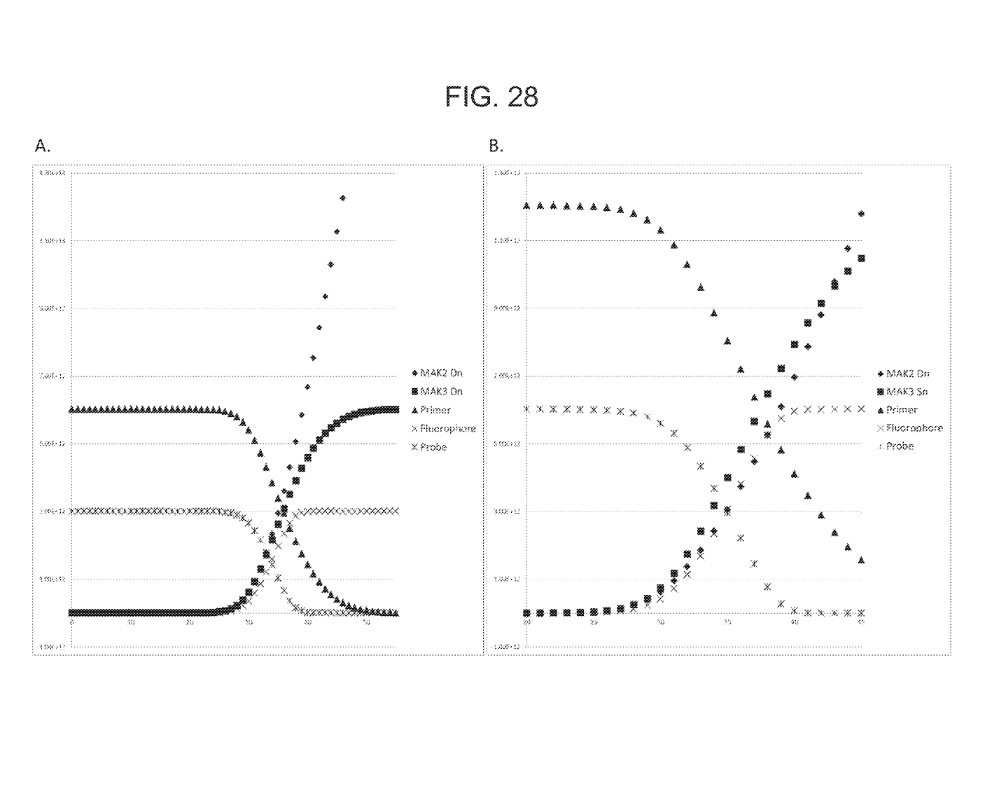

This invention discloses six examples of methods for finding such reference points called Fₘₐₓ_{_}_{curvature}, Fₘₐₓ_{_}_{second}_{_}_{derivative}, F_{inflection}, Fₘᵢₙ_{_}_{second}_{_}_{derivative}, Fₘᵢₙ_{_}_{curvature}. and Fₘₐₓ. FIG. 20 shows the approximate locations of the six intrinsic reference points. qPCR data from different instruments generally have different signal-to-noise. As a result, determining the positions of the intrinsic reference points from the analysis of raw data can result in significant error. Thus, the preferred embodiment for determining the intrinsic reference points is to fit the qPCR data to a smooth non-linear function and then to perform analytical or numerical derivatives on that function, rather than to perform numerical differentiation on the raw data. In some embodiments the data are truncated to remove data points at the beginning or end of the curve or even in the middle of the curve (if spurious points are evident).

One choice for a smooth curve that fits the raw data is the asymmetric log-logistic function described by Spiess and coworkers (Spiess, A.-N., Feig, C. and Ritz, C. “Highly accurate sigmoidal fitting of real-time PCR data by introducing a parameter for asymmetry”, BMC Bioinformatics 2008, 9:221-222.). The asymmetric log-logistic is given by Eqn. 25:

$${F}_{N} = M \times N + {F}_{b} + \frac{{F}_{max} - {F}_{b}}{{( 1 + {e}^{q \times ( log ( N ) - log ( r ) )} )}^{s}} \qquad Eqn . 25$$

where F_{N}=the fluorescence at cycle N, M=the slope of background fluorescence, F_{b}=the Y intercept for background fluorescence, Fₘₐₓ=the maximum fluorescence, q controls the steepness or abruptness of the elbows, r approximates the cycle number (i.e. x-axis value) corresponding to the inflection point of the curve, and s accounts for asymmetry of the bends in the elbows (Symmetric bends correspond to s=1).

Determining the proper log-logistic function. For a given qPCR dataset (i.e. the fluorescence vs. cycle data), the best-fit log-logistic function is determined by performing a Marquart-Levenberg non-linear least squares fit (see, e.g., Numerical Recipes in C, The Art of Scientific Computing, Cambridge University Press). This least squares fitting is performed on either the complete dataset of a truncated dataset (i.e. to remove excessively noisy points). This best fit function can then be differentiated and further analyzed to determine the intrinsic reference points as described below.

Determining the six intrinsic reference points. There are several ways to analyze the log-logistic function to determine the intrinsic reference points. The Fₘₐₓ is a direct output of the best-fit log-logistic function. The other five intrinsic reference points are determined using calculus or numerical optimization methods. The first method is to analytically determine the first, second, and third derivatives of the function. The inflection point is the cycle number where the second derivative of the log-logistic function equals zero.

III.a. Inflection Point

In certain embodiments of the technology of the invention, a preferred implementation of intrinsic referencing is to use the fluorescence at the inflection point in the qPCR curve, F_{inflection}. The “intrinsically referenced F(D₀)” is designated by a prime: F′(D₀), which is simply the ratio of the F(D₀) and F_{inflection} as follows:

$${F}^{′} ( {D}_{0} ) = \frac{F ( {D}_{0} )}{{F}_{inflection}} \qquad ( 26 )$$

where F_{inflection} is the fluorescence at the inflection point in the PCR curve (i.e. the point where the second derivative of the curve equals zero). Why does dividing by F_{inflection} help? The fluorescence of a sample is given by the following relationship:

F=εb[c]×φ_{F}×φ_{Q}×I (27)

where ε is the absorbance extinction coefficient, b is the pathlength, [c] is the sample concentration, φ_{F} is the fluorescence quantum yield, φ_{Q} is the fluorescence quenching, and I is an instrument dependent constant (that is different for different wells on one machine or different machines). The sample concentration is related to the number of copies of DNA at cycle zero, D₀, as follows:

$$[ c ] = \frac{{D}_{0}}{V \times {N}_{A}} \qquad ( 28 )$$

where V is the sample volume in liters and N_{A} is Avogadro's number (6.022141E23). Now we substitute Eqns. 27 and 28 into Eqn. 26 with cancelation of V and N_{A} to get:

$${F}^{′} ( {D}_{0} ) = \frac{F ( {D}_{0} )}{{F}_{inflection}} = \frac{ɛ {bD}_{0} \times {φ}_{F} \times {φ}_{Q} \times I}{ɛ {bD}_{inflection} \times {φ}_{F} \times {φ}_{Q} \times I} = \frac{{D}_{0}}{{D}_{inflection}} \qquad ( 29 )$$

Except for the DNA copy numbers, notice how all the other factors cancel, including the instrument dependent effects, I, and the fluorophore dependent effects (i.e., extinction coefficient, quantum yield, and quenching). Next, we apply equation 29 to the case of a single copy of DNA:

$${F}^{′} ( {D}_{1 copy} ) = \frac{F ( {D}_{1 copy} )}{{F}_{inflection}} = \frac{ɛ b \times 1 \times {φ}_{F} \times {φ}_{Q} \times I}{ɛ {bD}_{inflection} \times {φ}_{F} \times {φ}_{Q} \times I} = \frac{1}{{D}_{inflection}} \qquad ( 30 )$$

where we used the fact that by definition D_{1 copy}=1. As used herein, the terms “D_{1 copy}” and “D₀=1 copy” are used interchangeably to refer values for D from reactions having a single copy at cycle 0. Equation 30 has the surprising property that the intrinsically referenced fluorescence at the inflection point, F′(D_{1 copy}), involves a fixed number of copies of DNA. Recalling our analogy to the counting of apples, where to count the apples we weighed the apples including the basket, subtracted the basket, and then divided by the weight of 1 apple. Similarly, we now take the F′(D₀) (which already has the background subtracted) and divide by F′(D_{1 copy}), which should reveal the number of copies of DNA at cycle zero, D₀, as shown in Eqn. 31:

$${D}_{0} = \frac{{F}^{′} ( {D}_{0} )}{{F}^{′} ( {D}_{1 copy} )} = {D}_{inflection} \times {F}^{′} ( {D}_{0} ) \qquad ( 31 )$$

We recognize immediately that Eqn. 31 is simply the rearrangement of Eqn. 29, but now we realize from Eqn. 30 that D_{inflection} can be determined experimentally by simply the reciprocal of F′(D_{1 copy}). Equation 31 reveals the origin of the term “counting PCR” in which literally the number of copies of DNA at cycle zero is obtained by dividing the total fluorescence for all copies divided by the fluorescence for 1 copy.

Next, we recognize by rearranging Eqn. 28 that the number of copies of DNA at the inflection point, D₀, is directly proportional to the volume of the PCR reaction (presuming that the concentration of all chemicals in the reaction stay the same) and the proportionality constant, U, which depends upon other factors that will be described below.

$${D}_{inflection} = U \times V = \frac{1}{{F}^{′} ( {D}_{1 copy} )} \qquad ( 32 )$$

Combining equations 31 and 32 gives:

D₀=U×V×F′(D₀) (33)

Eqn. 33 is an important equation since it relates the desired D₀ to easily determined quantities (namely V and F′(D₀)) and the U is a constant that is assay dependent.

### Other Methods for Intrinsic Referencing.

The inflection point is not unique as a method for performing intrinsic referencing. The inflection point happens to be a point that can be easily identified on the PCR curve regardless of the starting number of copies of DNA, D₀, used in a qPCR reaction. Thus, any alternative easy to identify points would also work for scaling the F(D₀) and we have identified five alternatives to the F_{inflection point}: Fₘₐₓ, F_{max second derivative}, Fₘₐₓ_{_}_{curvature}, Fₘᵢₙ_{_}_{curvature}, and Fₘᵢₙ_{_}_{second}_{_}_{derivative}. Notably, other points on the curve that are arbitrary multiples (equal to 1 or less) of the four points are also suitable reference points (for example 0.5*F_{inflection} or 0.86921*F_{max second derivative} or 0.6*Fₘₐₓ are also a suitable reference points). The four methods for intrinsic referencing have different inherent assumptions that underlie their use. The point of maximum curvature occurs at the lowest cycle number, and as a result it depends the least on other variables and is mostly determined by the shape of an exponential amplification curve. The maximum of the second derivative occurs at a higher cycle number than max curvature and thus it is dependent upon the exponential amplification and upon the damping in PCR efficiency that occurs due to the value of K in the MAK2 model. The inflection point is influenced by the exponential amplification, the PCR efficiency at each cycle, as well as the consumption of reagents in the qPCR reaction (including the depletion of NTPs, primers, and in extreme cases concentration of enzyme). The Fₘₐₓ method, which uses the fluorescence at saturation of the curve (i.e. in the ideal limit of infinite cycle number) depends upon all of the above factors plus the enzyme concentration, NTP concentration, and Magnesium concentration. In practice, the Fₘₐₓ method can be difficult to apply in cases where the number of cycles acquired is insufficient to extrapolate to deduce the maximum fluorescence value (often there is a linear upper baseline that makes it not obvious what the maximum Fluorescence value is). The Fₘₐₓ method is particularly problematic in cases where the D₀ is 1 copy and thus saturation may not be achieved in a typical qPCR number of cycles (typically 40 cycles are used, but even with 55 cycles the reaction may not achieve full saturation). Nonetheless, we have successfully applied the Fₘₐₓ method and obtain results that are only slightly worse than the inflection point method.

For the example GAPDH data set (see Examples), we obtained percentage standard deviations in relative quantification as follows for the MAK2 model of Boggy and Woolf:

| Method | % Standard Error |
|---|---|
| F(D₀) | 15.5 |
| F_{inflection} | 4.9 |
| Fₘₐₓ | 5.9 |
| Max 2^{nd} Derivative | 8.7 |
| Max Curvature | 9.1 |
| Min 2^{nd} Derivative | ND* |
| Min Curvature | ND* |

*Where ND means not determined.

The Table above gives typical errors for the six different reference points. These data reveal that the inflection point method has the lowest % standard error when sufficient data are available. The Fₘₐₓ method is the next most accurate method, though it is subject to the problem that the Fₘₐₓ may be difficult to determine in insufficient cycles are acquired. Nonetheless, the Fₘₐₓ method can be the method of choice for samples that have high starting concentration. The inflection point method has the drawback that the reaction must have a low enough concentration so that some lower baseline is obtained and the inflection point occurs at a cycle number greater than cycle 5. Both the maximum second derivative method and the maximum curvature method are very demanding on the signal-to-noise in the qPCR data and thus they have lower performance than the inflection point and Fₘₐₓ methods. In addition, the use of the inflection point method means that the process of counting copies of DNA uses only data that are a few cycles after the inflection point, so that fewer cycles need to be experimentally acquired. The minimum number of cycles to collect to ensure that saturation is achieved even for a single copy of DNA depends upon the volume of the sample (35 cycles is sufficient for reactions of 10 nL, while 50 cycles is sufficient for reactions of 20 μL). The minimum curvature, and the minimum second derivative points perform less well and require qPCR data that extend well beyond (i.e. typically at least five points) the inflection point.

For the primer depletion model, the errors in F(D₀) are already quite small (typically below 8% for copy numbers above about 1000 copies, wherein the error is largely determined by the user pipetting error) and F′(D₀) using the inflection point is only slightly smaller than the error in F(D₀). This smaller error in F(D₀) is a consequence of the fact that the primer depletion model is superior to the MAK2 model of Boggy and Woolf. Nonetheless, intrinsic referencing retains the strong advantage of producing machine independence.

### Numerical Methods for Determining the Maximum Curvature, Maximum Second Derivative, and Inflection Point.

### III.b Max Curvature:

Within the range of cycles used for fitting, qPCR CopyCount forms an interpolating cubic spline between either a) the raw data points; or b) the fitted data points. The qPCR CopyCount process for copy counting from qPCR signals derives a unique x value (i.e. cycle number) where the curvature is maximum by an iterative or analytical approach.

Iterative approach. In an initial pass to examine intervals at their endpoints, qPCR CopyCount finds the interval endpoint position with the greatest curvature. This is accomplished by performing either of two different methods using either three points or five points (i.e. cycles of pPCR):

Let h be the x distance (in cycles) between two points x(i) and x(i+1).
a) If using three points numbered i through i+2, then qPCR CopyCount approximates the first derivative for central position i+1 as:

y′=(f(i+2)−f(i))/(2*h)
and qPCR CopyCount approximates the 2nd derivative for central position i+1 as:

y″=(f(i+2)−2*f(i+1)+f(i))/(ĥ2)
then qPCR CopyCount approximates the first derivative for central position i+2 as:

y′=(−f(i+4)+8*f(i+3)−8*f(i+1)+f(i))/(12*h)
and qPCR CopyCount approximates the 2nd derivative for central position i+2 as:

y″=(−f(i+4)+16*f(i+3)−30*f(i+2)+16*f(i+1)−f(i))/(12*ĥ2)

qPCR CopyCount then finds the maximum curvature point by a process of iterative bisection. For each iteration, it divides h (i.e. the x distance between points) in half and reevaluate the curvature at the midpoints of the regions above and below the current maximum. If either of the midpoints forms a new maximum, then the algorithm re-centers around that new maximum. Otherwise, the algorithm will retain the current maximum and continue narrowing.

The iterative looping stops when we have reached a sufficiently refined value of x for the maximum curvature.

Analytical approach: qPCR reactions involve an exponential amplification process in which at the beginning cycles the process is nearly perfectly exponential, but later in the process (i.e. as the curve gets closer to the inflection point), then the qPCR amplification process starts becoming less efficient and thus no longer a perfect exponential growth. This observation suggests that if data from the qPCR reaction are truncated to include only those points that are well below the inflection point, then an ideal exponential function is a good approximation of the actual qPCR amplification. For practical purposes, analytical fitting using an exponential function truncates the data at 3-5 points below the inflection point (the method for finding the inflection point is described elsewhere in this patent). For example, if the inflection point occurs at cycle 30, then points 1-27 or 1-26 or 1-25 would be used (the interval with the best fit to an exponential function is retained). As an approximation to the exponential amplification that occurs in qPCR (i.e. neglecting the decreasing efficiency that occurs with each cycle) qPCR CopyCount applies the function: y=a*2^{x}, where a=F(D₀), and y is the fluorescence at cycle n, F(Dₙ). Note that since this is an analytical function, non-integer cycles, n, are allowed. Note that it is important to subtract the linear background from the data before performing the exponential fitting analysis. The background slope and intercept are obtained from the MAK2 fitting with four parameters (F_{b}, Fₘ, F(D₀), and K) described above.

For a function y=a*2^{x}, the curvature is given by:

$$Curvature = \frac{{y}^{″}}{{( 1 + {( {y}^{′} )}^{2} )}^{3 / 2}} \qquad ( 34 )$$

then the first derivative is:

$${y}^{′} = \frac{d y}{d x} = a \times {2}^{x} \times Ln ( 2 )$$

and the 2nd derivative is:

$${y}^{″} = \frac{{d}^{2} y}{d {x}^{2}} = a \times {2}^{x} \times {( Ln ( 2 ) )}^{2}$$

and the curvature is:

$$Curvature = \frac{a \times {2}^{x} \times {( Ln ( 2 ) )}^{2}}{{( 1 + {( a \times {2}^{x} \times Ln ( 2 ) )}^{2} )}^{3 / 2}} \qquad ( Eqn . 35$$

To find the cycle number where the curvature is maximum, take the first derivative of the curvature with respect to the cycle number, sets the derivative to zero and solves for x (i.e. the cycle number) as follows:

solve:

$$\frac{d ( curvature )}{d x} = \frac{d}{d x} ( \frac{a \times {2}^{x} \times {( Ln ( 2 ) )}^{2}}{{( 1 + {( a \times {2}^{x} \times Ln ( 2 ) )}^{2} )}^{3 / 2}} ) = 0$$

giving

$$x = \frac{Ln ( \frac{1}{2 {{a}^{2} ( Ln ( 2 ) )}^{2}} )}{Ln ( 4 )} \qquad ( Eqn . 36 )$$

where a=F(D₀). Note that the maximum curvature method can only be applied if the maximum curvature occurs above a cycle number of zero (i.e. the maximum concentration of DNA at cycle zero must be below about 500,000 copies of DNA per nL or qPCR reaction).

By either method for determining x (i.e. the iterative approach or the analytical approach), qPCR CopyCount then uses that x value (where the curvature is maximum) with the cubic spline method (preferably applied to the Y values from the analytical fit or alternatively (but less preferred) to the original Y values from the raw data—i.e. the raw fluorescence values minus background) to calculate a corresponding y value. This y value is the Fₘₐₓ_{_}_{curvature} that is used for the intrinsic referencing.

In an alternative less-preferred embodiment, qPCR CopyCount performs the exponential fitting to the original qPCR data interval without subtracting the background. Then the y-value corresponding to the maximum curvature contains the background fluorescence. If this alternative is performed, then the Fₘₐₓ_{_}_{curvature} that is used for the intrinsic referencing is obtained by taking the y value corresponding to maximum curvature and subtracting the background (Fₘ*x+F_{b}).

### III.c Max Second Derivative

Alternative embodiments of the maximum second derivative method include the following:
a) iterative evaluation of cubic splines interpolated between the modeled fit values.
b) iterative evaluation of cubic splines interpolated between a simulated MAK2 curve.
c) analytical evaluation of a polynomial interpolation between 5 points of a simulated MAK2 curve.
d) analytical evaluation of a polynomial interpolation between 5 points of the raw data values.

In certain preferred embodiments, method d is applied.

Both of the iterative evaluation methods (a or b) use iterative refined searching to narrow down to the maximum second derivative. They differ only in that method “a” is based on the data points of the modeled curve produced by a nonlinear regression through the data, whereas method “b” is based on data points produced through a simulation of the MAK2 curve, where Yn+1=K*log(1+Yn/K).

The iterative approach can be described using the particular case of the context of the MAK2 curve (method “b”). The version using values from the fit curve works equivalently. The MAK2 case (i.e. method “b”) proceeds as follows.
1. A series of values can be calculated based upon the MAK2 model, where Yn+1=K*log(1+Yn/K).
2. The y values between these points, i.e. within each cycle interval, can be estimated by using interpolation. This can be done using a cubic spline or some other interpolating function that provides a means for generating arbitrary intermediate y values, given an x value.
3. Within any 3 points, the 2^{nd} derivative of the central point central position i+1 can be estimated as y″=(f(i+2)−2*f(i+1)+f(i))/(ĥ2), where h is the x distance (in cycles) between two points x(i) and x(i+1).
4. An initial pass through the data points can be used to locate a preliminary point with a maximum 2^{nd} derivative. That point and its neighboring point to either side form the region of interest for iterating to refine the estimate for the point of maximum 2^{nd} derivative.
5. On each iteration, the second derivative is evaluated for two mid-points that occur in between the central current maximum point and each of its neighboring points. If either of these has the greatest 2^{nd} derivative so far, it becomes the center of a smaller interval in the next iteration. On the other hand, if neither of these new midpoints has a superior 2^{nd} derivative, then the current central point with the maximum 2^{nd} derivative is retained while the width of the interval is narrowed.
6. With each iteration, the width on the x axis of the interval being considered is half the width of the previous iteration's interval. The looping can exit when the estimate of x and y for the maximum 2^{nd} derivative drop below some required threshold.

By contrast with the iterative methods, either of the analytical methods (c or d) allows forming a polynomial interpolating function, making it possible to analytically describe the expression for the maximum second derivative. This allows directly calculating the x value for the estimated maximum second derivative. From this x value, the corresponding y can also be produced.

For version c using MAK2, we can follow the following strategy.
1) Generate a series of data points using MAK2 simulation, where Yn+1=K*log(1+Yn/K);
2) Find an estimated preliminary maximum 2nd derivative. As noted previously, using three data points one can approximate the 2nd derivative for central position i+1 as:

y″=(f(i+2)−2*f(i+1)+f(i))/(ĥ2), where h is the x distance (in cycles) between two points x(i) and x(i+1);
3) Identify the five data points centered on that preliminary maximum;
4) Use polynomial interpolation to form a known 4th order curve. Given five coefficients, cof[4] through cof[0], we have

y=cof[4]*x̂4+cof[3]*x̂3+cof[2]*x̂2+cof[1]*x̂1+cof[0]*x̂0

y′=4.*cof[4]*x̂3+3.*cof[3]*x̂2 +2.*cof[2]*x̂1+cof[1]*x̂0

y″=12.*cof[4]*x̂2+6.*cof[3]*x̂1+2.*cof[2]*x̂0

y′″=24.*cof[4]*x̂1+6.*cof[3]*x̂0=0

x at the max 2^{nd} derivative=−6.*cof[3]/24.*cof[4]
5) Use the coefficients of that polynomial function to find a true 2nd derivative maximum; and
6) For the maximum's x, find the curve's corresponding y value.

Method d is similar to method c, except that the raw data points are used and the 4^{th} order polynomial function is based on the five raw data points centering on the point with the preliminary maximum second derivative.

### The Inflection Point Method:

qPCR is a process that occurs using integer cycle numbers. However, the inflection point in the curve that is needed for intrinsic referencing is the inflection point for an ideal continuous curve. Thus there is a need to perform data interpolation to determine the non-integer cycle number where the inflection point occurs.

Methods for determining the inflection point comprise the two alternative methods, as follows:

### Method One: Interpolating Between Raw Data Points

1. For estimating inflection point from the raw data, we interpolate between the raw data points. This can be done using a cubic spline or some other interpolating function that provides a means for generating arbitrary intermediate y values, given an x value.
2. The program determines the second derivative values y″ at the cycle endpoints. For some methods of constructing the cubic spline, this information can be a byproduct of that process. [Example: the spline function given by Numerical Recipes, by Press, Teukolsky, Vetterling, and Flannery, Cambridge University Press]. Otherwise, as noted previously, using three data points one can approximate the 2nd derivative for central position i+1 as:

y″=(f(i+2)−2*f(i+1)+f(i))/(ĥ2)
3. From among any intervals that transition from a non-negative 2nd derivative to a negative 2nd derivative at the endpoints of the interval, we select the interval with the greatest increase in y between the endpoints. (This interval contains the inflection point.)
4. The program performs a binary search, iteratively bisecting the current interval, to narrow down to the inflection point. By determining the second derivative at the midpoint, one can choose a new interval half the size of the previous interval that preserves the property of a transition from a nonnegative second derivative to a negative second derivative. For this purpose, the y values can be supplied by the interpolating function (e.g. cubic spline) and the second derivative values can be estimated using the three point method just mentioned in step 2.

### Method Two: Regression Fitting to the Data

As an alternative to fitting the data by interpolation, such as with a cubic spline, one may also fit a curve to the raw data, e.g., with a nonlinear regression. Two candidates for this procedure are the five-parameter logistic curve and the preferred five parameter log-logistic curve. As indicated by Boggy and Wolf [A Mechanistic Model of PCR for Accurate Quantification of Quantitative PCR Data, PLoS ONE, August 2010, Volume 5, Issue 8.]:
“The equation for the five-parameter log-logistic function is:

Fn=Fb+(Fmax−Fb)/(1+ĉ(q*(log(n)−log(r))))̂s (37)
where Fn, Fb, and Fmax are the fluorescence at cycle n, background fluorescence, and maximum fluorescence respectively; and parameters q, r, and s adjust the shape of the curve. The logistic model is identical to the log-logistic model in equation (37) except the (log(n)−log(r)) term is replaced by (n−r).”

Within this method, the nonlinear regression function that fits the data replaces the interpolating function of Method One.

### Method for Determining the Inflection Point:

In some embodiments, the inflection point can be obtained by performing differentiation of the fit log-logistic curve:

1^{st} derivative=qs(F_{b}−Fₘₐₓ)r^{-q}N^{q-1}(r^{-q}N^{q}+1)^{-s-1}+M

2^{nd} derivative=qs(Fₘₐₓ−F_{b})r^{-2q}N^{q-2}(r^{-q}N^{q}+1)^{-s-2}((qs+1)N^{q}−(q−1)r^{q})

In other embodiments, the first, second, and third derivatives may be determined numerically.

In certain embodiments, each of the five remaining reference points can be determined by a bisection algorithm:

$$Curvature = \frac{{y}^{″}}{{( 1 + {( {y}^{′} )}^{2} )}^{3 / 2}} \qquad ( 34 )$$

Inflection point is where the second derivative=0 and is given by:

N for F_{inflection}=((q−1)r^{q}/(qs+1))

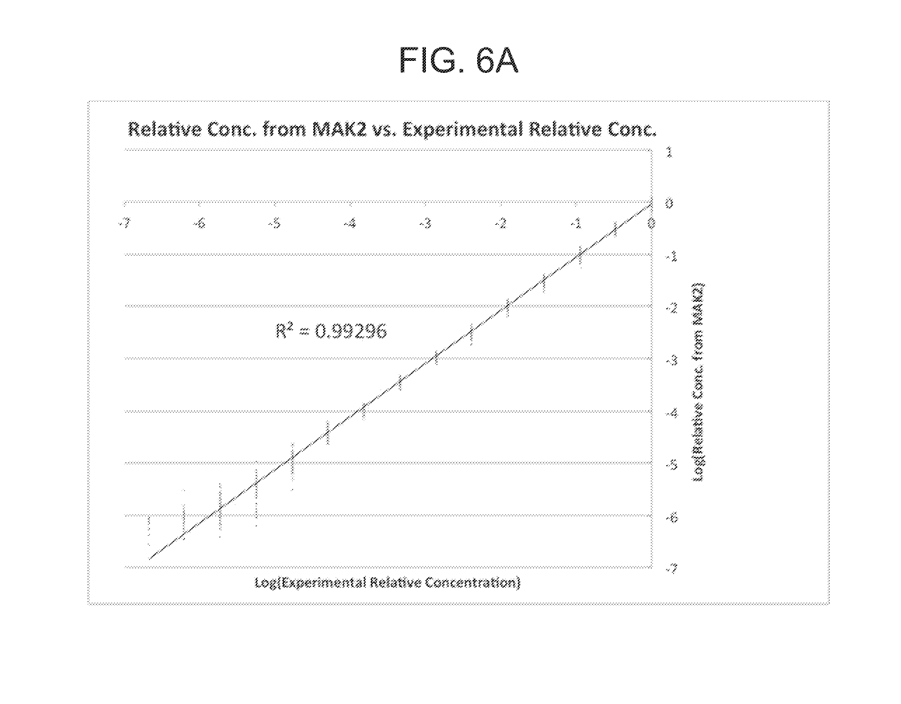

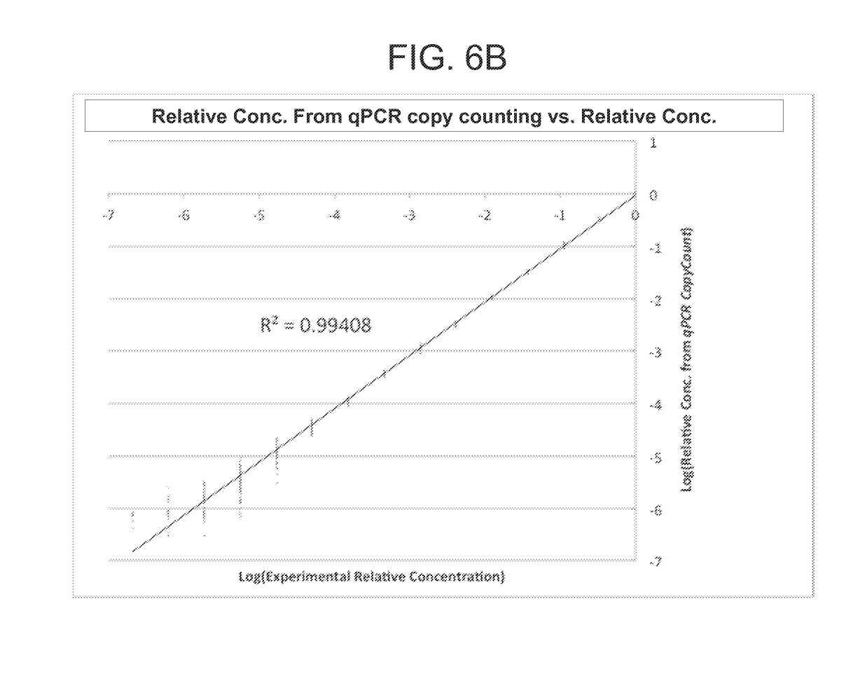

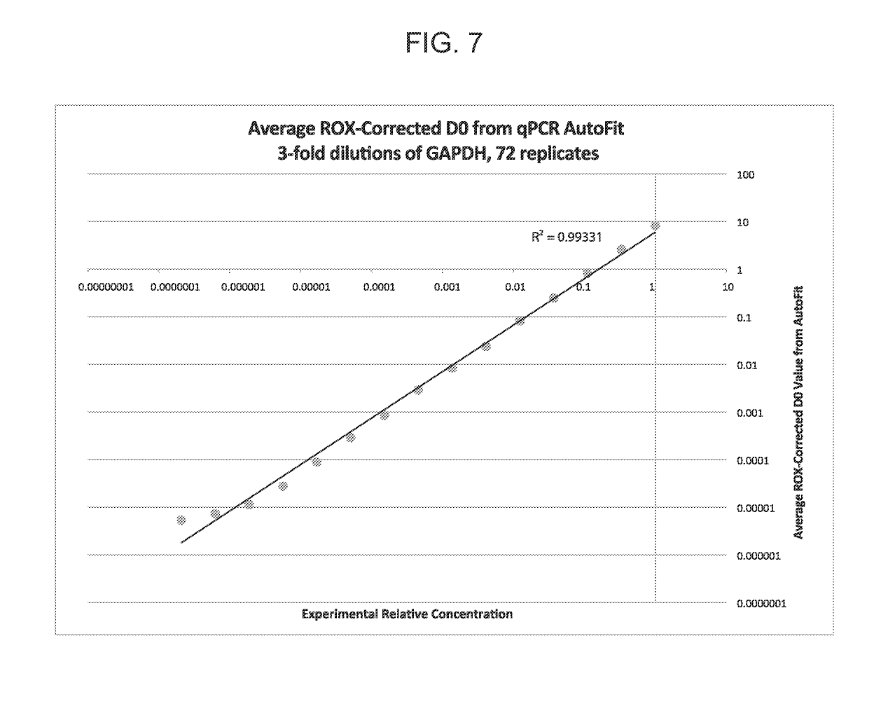

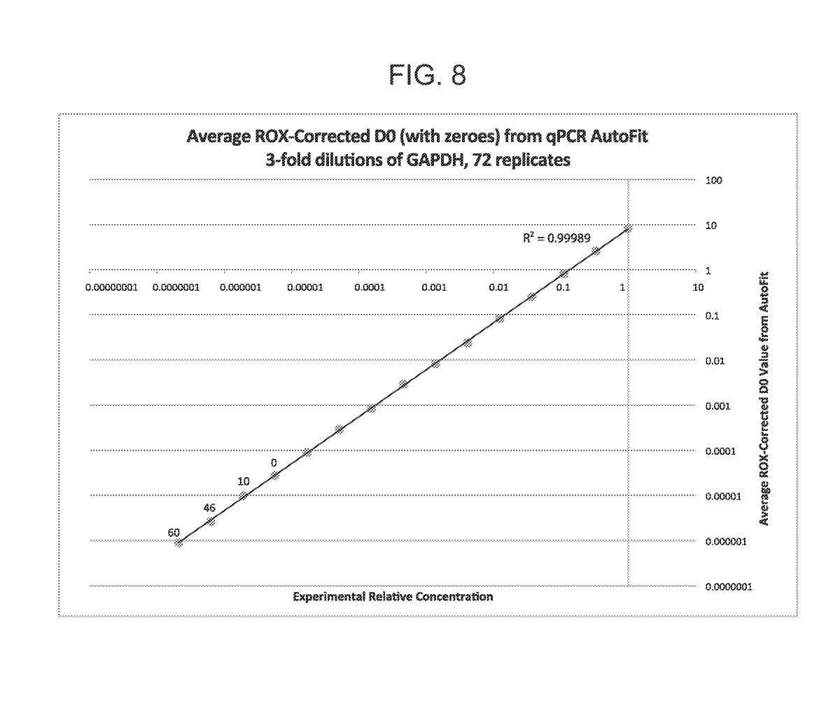

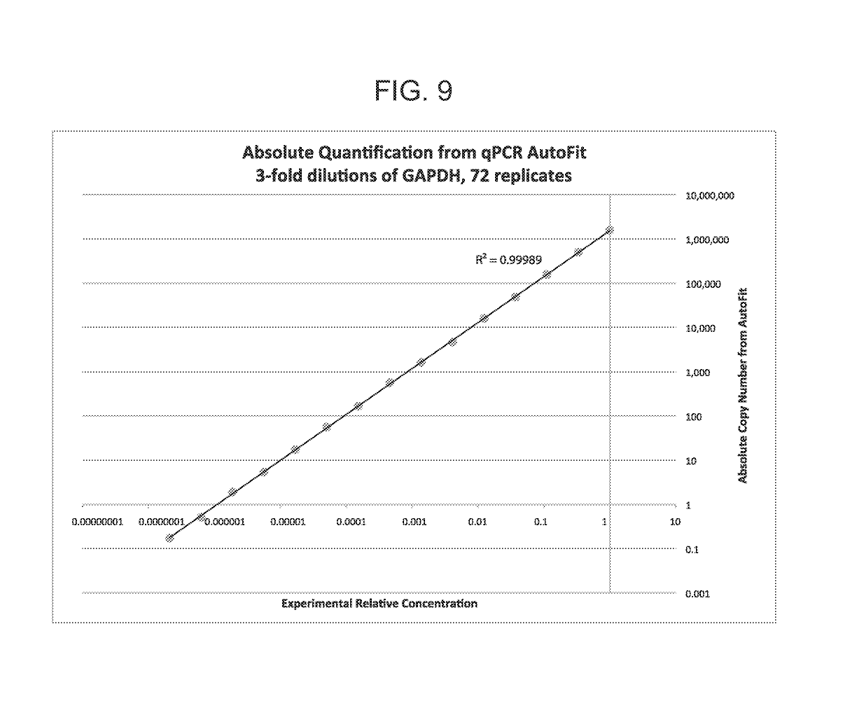

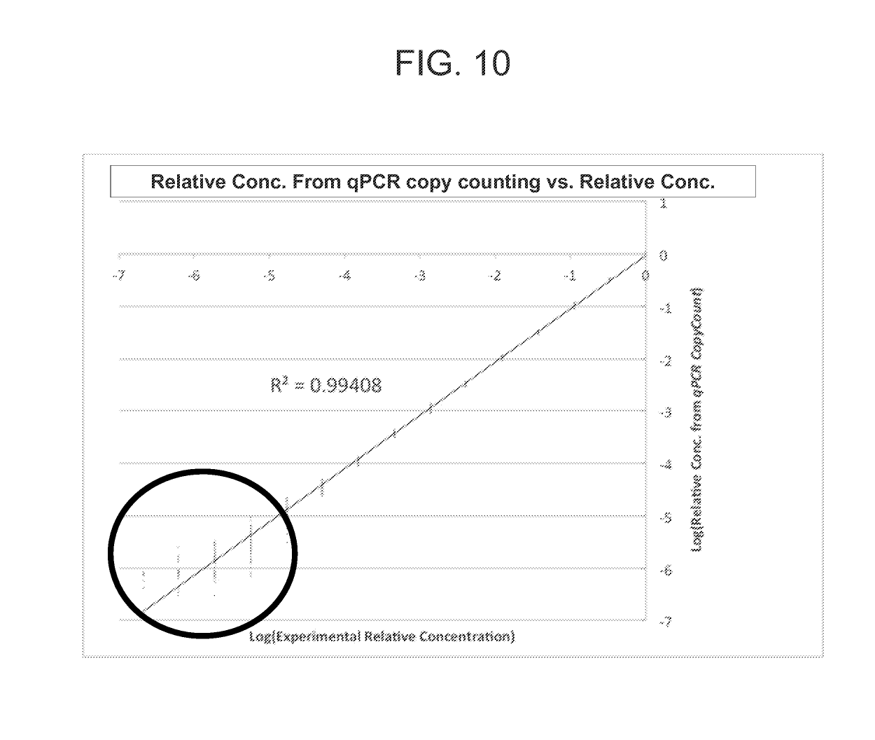

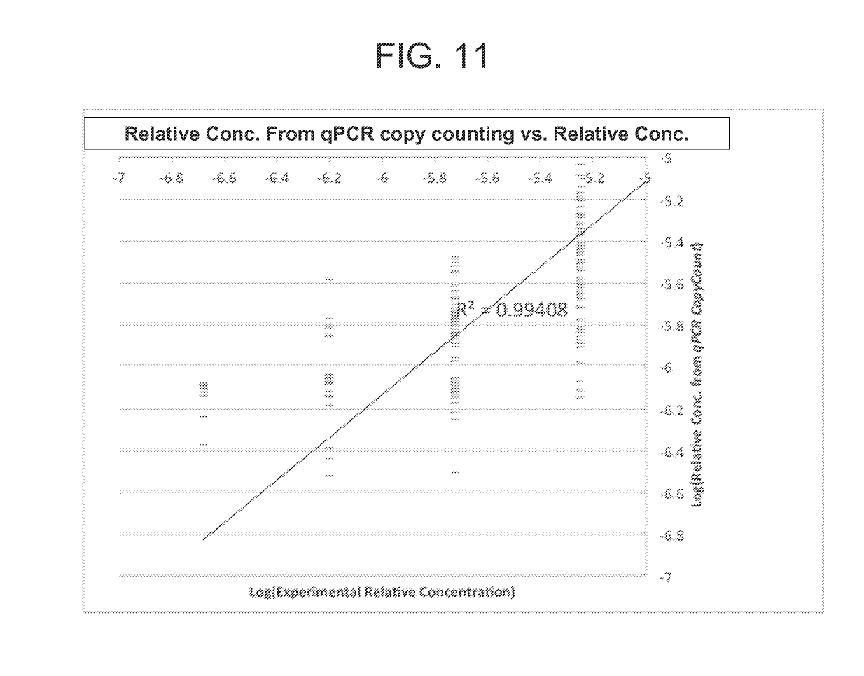

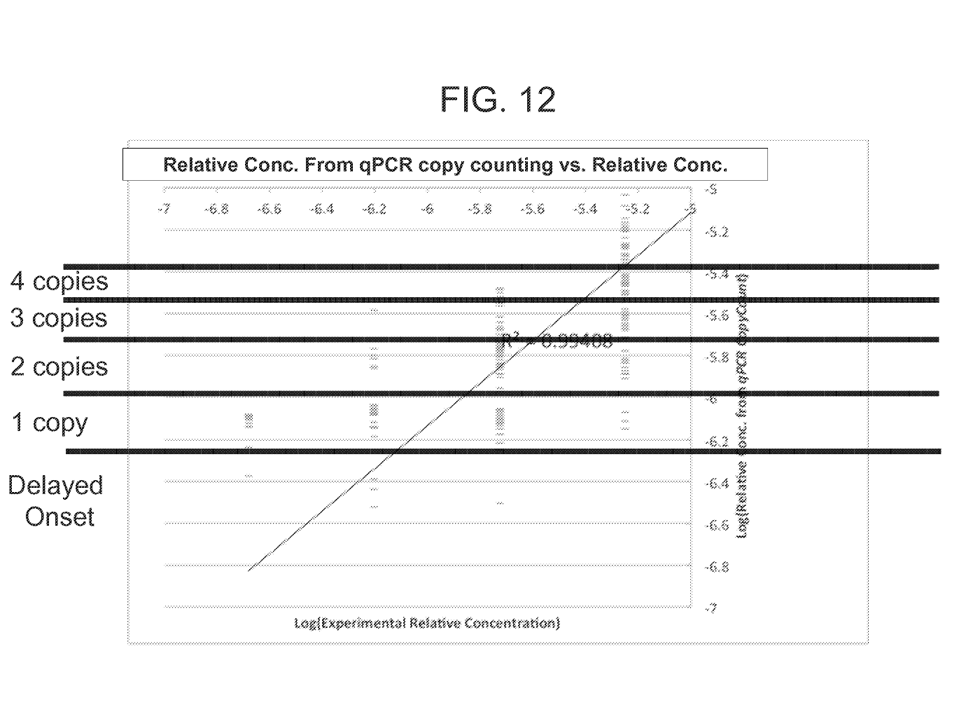

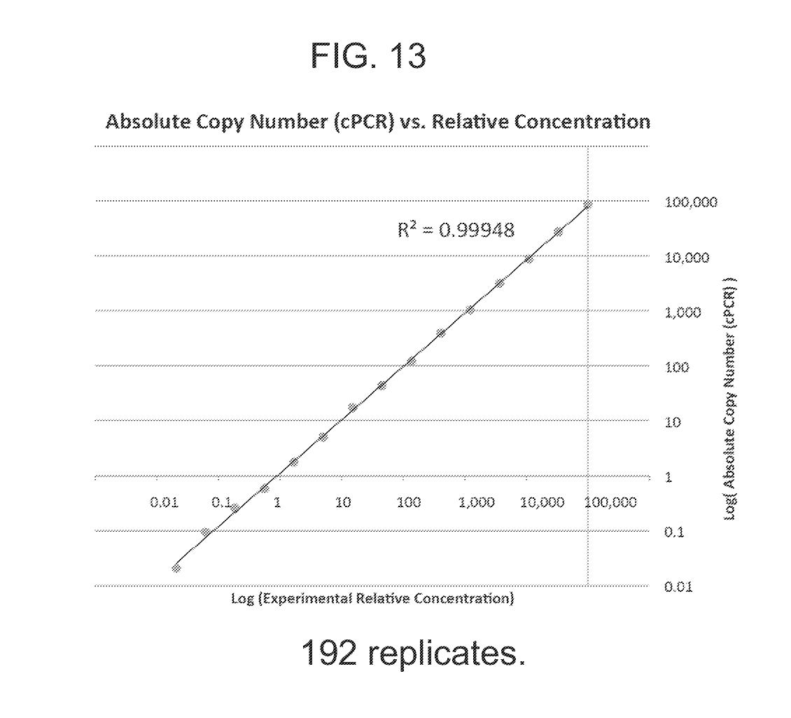

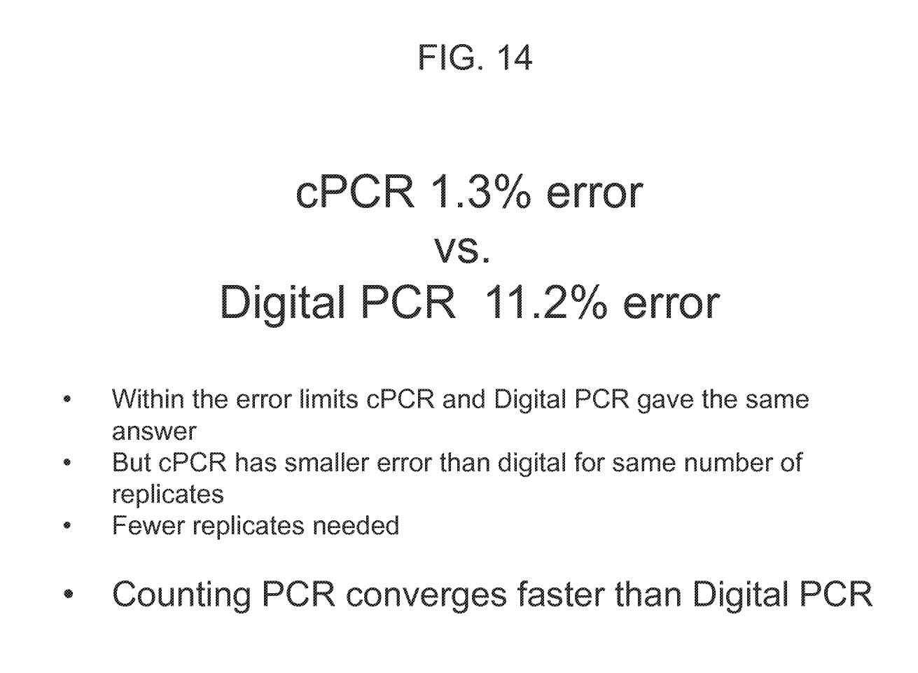

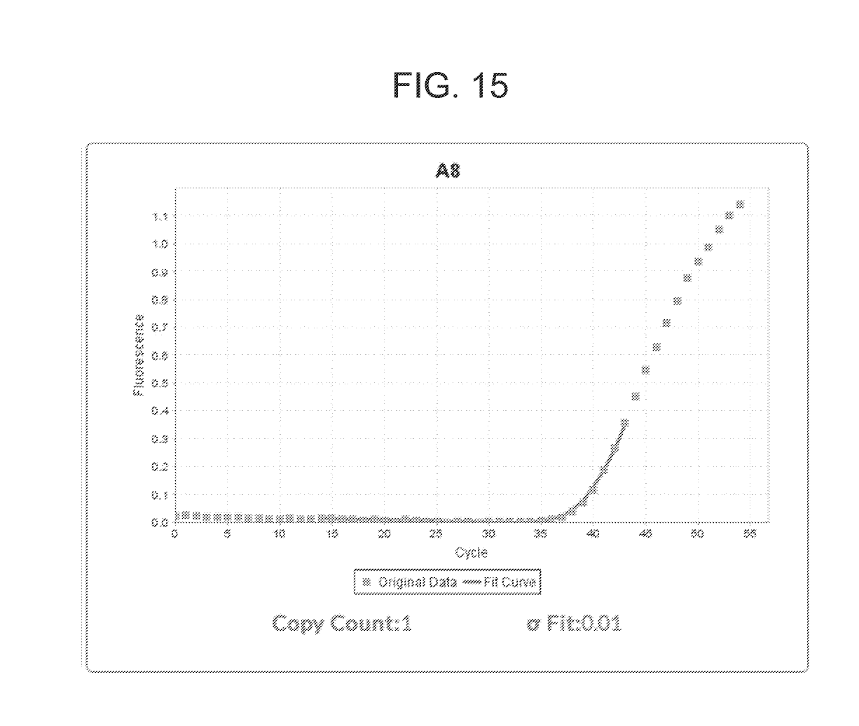

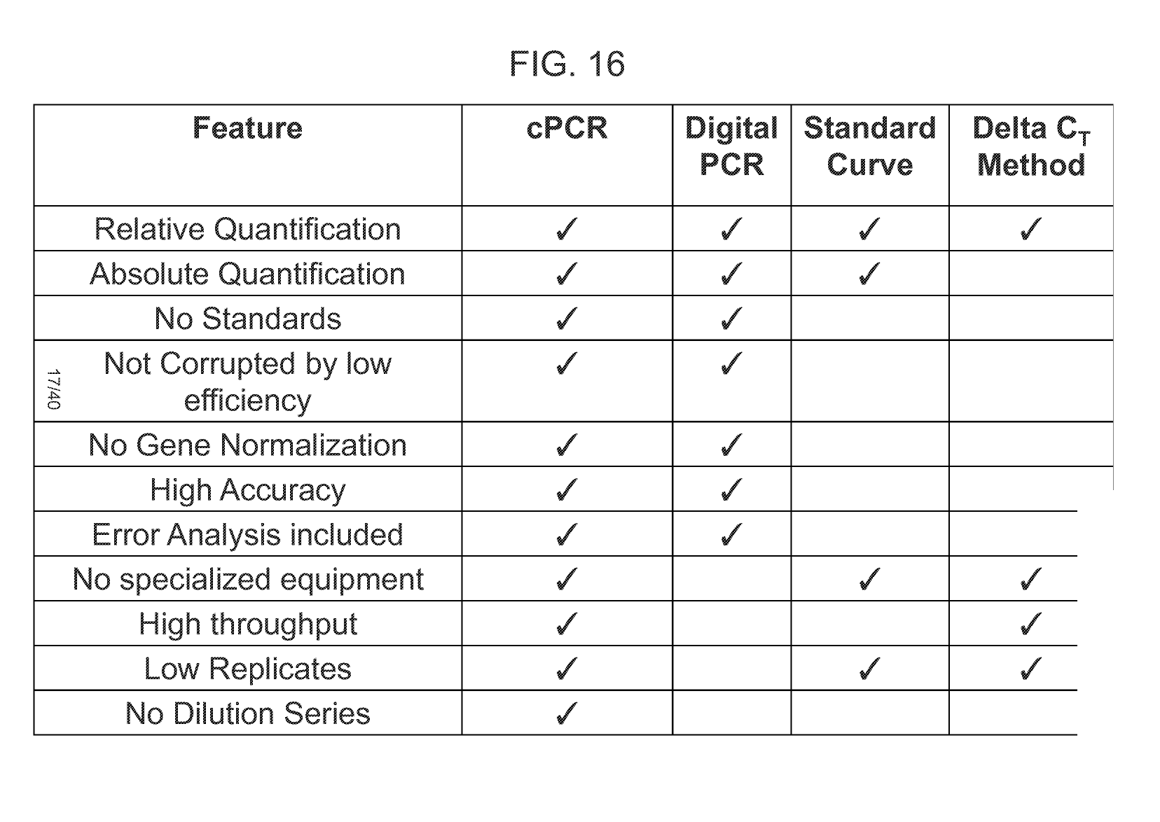

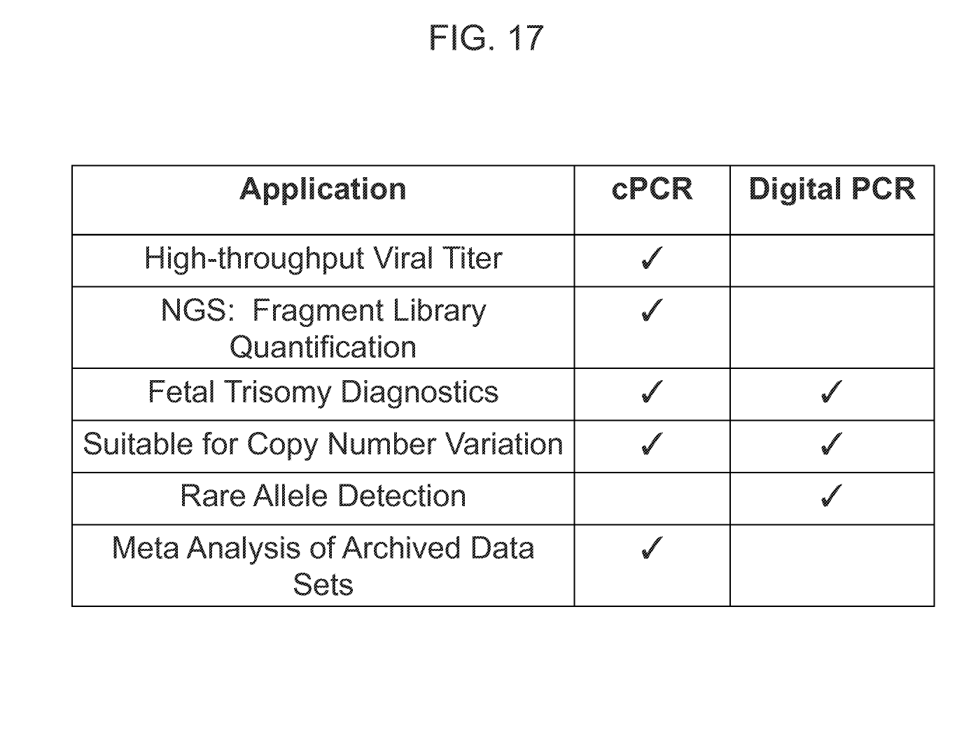

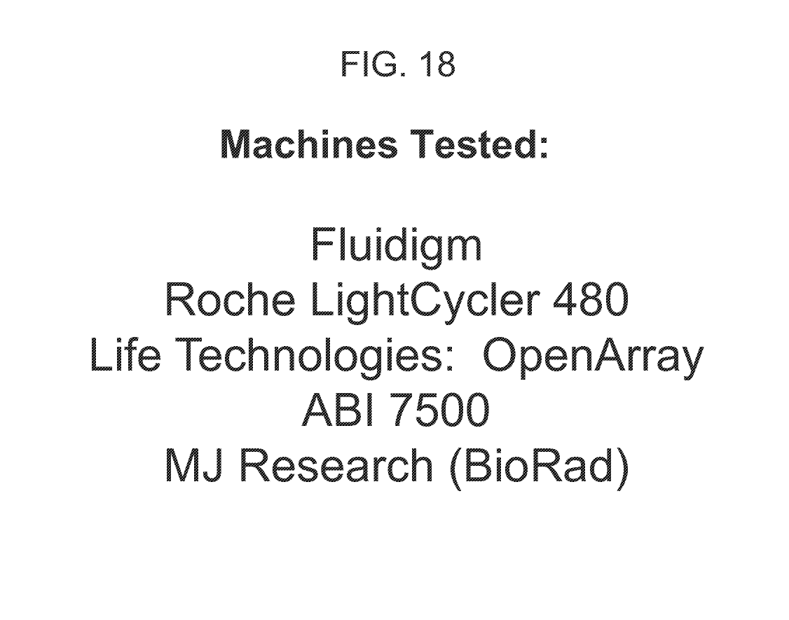

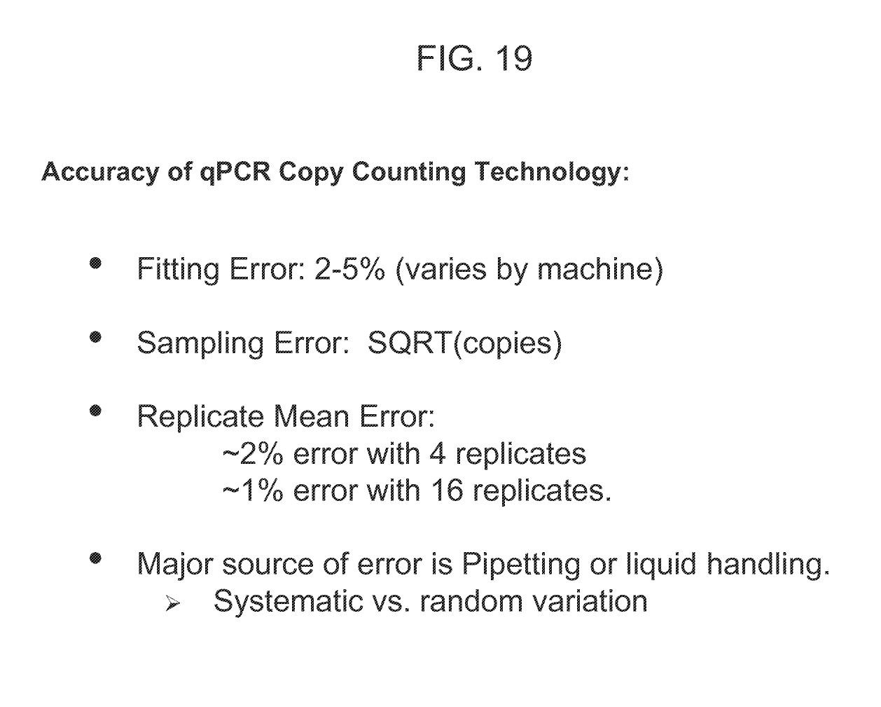

FIG. 9 shows the effect of applying the intrinsic referencing analysis to the same raw data set shown in FIG. 6. Compare the quality of these data to those in FIGS. 7 and 8 (i.e. with ROX correction). The intrinsic referencing has standard error that is as small as that from ROX correction.

### III.d Max

The Fₘₐₓ method retrieves the fluorescence of the maximum cycle number, Fₘₐₓ, and divides F(D₀) by that amount. This gives the following equation for F′(D₀):

$${F}^{′} ( {D}_{0} ) = \frac{F ( {D}_{0} )}{{F}_{ma x}} \qquad ( 38 )$$

Such intrinsic referencing to Fₘₐₓ removes scale information from the qPCR data and puts all qPCR curves on the same scale. This removes the same variations as ROX correction, namely variation in instrument illumination, pipetting errors, etc. Variants of the Fₘₐₓ method are to average several of the last few cycles of the qPCR curve or to fit the last several points to a linear equation, either process will lessen the contribution of noise that using the last cycle alone might have. Alternatively, several qPCR replicates of the same reaction may be run and averaged, or have the median taken, again with the goal of minimizing noise. Note that normalizing to the Fₘₐₓ is equivalent to normalizing at other cycle numbers that are some fraction of Fₘₐₓ.

By performing intrinsic referencing on each qPCR data set, relative quantification for different targets is determined as follows:

$$\frac{{D}_{o}^{A}}{{D}_{o}^{B}} = \frac{U \cdot {F}^{′} ( {D}_{o}^{A} )}{U \cdot {F}^{′} ( {D}_{o}^{B} )} = \frac{{F}^{′} ( {D}_{o}^{A} )}{{F}^{′} ( {D}_{o}^{B} )} \qquad ( 39 )$$

where U is a universal constant that is assay- and reaction-volume dependent, but is otherwise the same for all targets (including those with different dyes so that multiplexing is enabled). An important implication of the universal constant is that intrinsic referencing allows for universal calibration to deduce the absolute DNA copy number, D₀, as shown in equation (40):

D₀=U·V·F′(D₀) (40)

The technology of the present invention for counting copies of PCR products also provides an estimate of the standard deviation of F′(D₀), sigma_F′(D₀). The typical sigma_F′(D₀) determined from a single qPCR well is less than 5%. By performing replicates and averaging their values to obtain the mean, the sigma_mean_F′(D₀) can be reduced as follows:

$$sigma\_mean {\_F}^{′} ( {D}_{0} ) = \frac{{sigma\_F}^{′} ( {D}_{0} )}{\sqrt{N}} \qquad ( 41 )$$

where N is the number of replicates. With just 4 replicates, the error from the qPCR counting method is less than 3%, and with 16 replicates the error is less than 2%. Such relative quantification has significant applications for, e.g., non-invasive cancer diagnostics, fetal diagnostics, determination of copy number variations, SNP genotyping, expression profiling, and other applications.

### IV. CALIBRATION OF U

Since U is related to D_{inflection} according to equation 32, we recognize that since F′(D_{1 copy}) is instrument independent, then D_{inflection} and U are also instrument independent quantities. However, U does depend upon the chemical composition of the qPCR reaction (i.e. the assay). Thus, the calibration of U only needs to be performed one time for each new assay (i.e. each new design of primers and probes (or change in concentrations of primers and probes) and master mix). There is no need to perform such a measurement on each new sample or even for different instruments.

In certain embodiments, methods for calculating U comprise the following two methods: A. Empirical calibration of each assay, and B. Theoretical calibration.

IV.a. Empirical Calibration of U.

Equation 32 shows an embodiment of how to determine U experimentally. A complete protocol for the experimental determination of U for a given assay is given in Example 3: Two-step Calibration Procedure for determining U. The program then determines U from the calibration plate data by the following procedure given in the section: IV.d calibration plate methods (i.e. the “no boundaries” method, which is the preferred embodiment).
1. Dilute a sample to an approximate average of approximately 0.5 to 3.5 molecules of target DNA per well (e.g., as is commonly done for digital PCR)
2. Perform a qPCR experiment in volume V, and the fit with the MAK2 model to get F(D0) for all the qPCR wells.
3. Determine the F_{inflection} using the numerical methods described above.
4. Take the ratio of F(D₀) and F_{inflection} to get F′(D₀).
5. Average all of the F′(D₀) for all the wells including those with zero copies.
6. Analyze the Poisson distribution to determine the Mean copy number for the plate. In an alternative embodiment, the mean can be determined using digital PCR or by performing a standard curve (only one time for a given assay, it will never need to be run again).
7. Compute the fluorescence from one molecule using equation 50.
8. Use Eqn. 32 to calculate U. U=1/(F′(D₀=1 copy)*V)

It will be appreciated that other methods of determining F′(D_{1 copy}) described herein may also be used.

IV.b. Theoretical Calibration of U

In many applications (such as viral load determination, and some applications for fragment library quantification for next generation sequencing), an approximate absolute quantification (with error less than 30%) is sufficient and sample throughput is more important than accuracy. For such cases, using a general approximate U value is appropriate so that there is no need to perform the assay calibration given above. An alternative way to compute U is to understand the physical basis for U, and then compute it from first principles and given the chemical composition of the PCR reaction. Our current method can predict U within a standard error of 30% given the pdK, [primer], [probe], amplicon length, MGB y/n, and target double stranded y/n (U is independent of volume, but the reaction volume is needed to compute D₀). Other variables that affect U are the amplicon length (or more correctly the amplicon sequence), primer lengths (or more correctly the primer sequences), [NTPs], [Mg], and enzyme concentration, but these effects are mostly contained within the pdK parameter and the [NTPs] is approximately accounted for in the amplicon length parameter. The U_{inflection} depends most strongly on [probe] and [primers] which have an inherent error of about 10-15% due to inaccuracy in the UV absorbance extinction coefficients. This experimental inaccuracy in the [primer] and [probe] puts a lowers limit of about 15% for even a perfect theory for predicting U.

### U Prediction Method for TaqMan Based Detection:

The precise theory of how U depends upon the [primer], [probe], pdK, amplicon length, presence of MGB, and other factors is not known. Thus, we have developed empirical models that are called parameterized “heuristic_U equations”. Various mathematical equations were empirically tested and their parameters (i.e. adjustable coefficients) were determined by fitting a set of experimental U values that were acquired for 51 assays with systematically varied [primer], [probe], amplicon length, MGB y/n, and target double stranded y/n. The pdK parameter was derived from the fitting of the qPCR curves and averaging them (after removing outliers and not including those wells with zero copies of DNA) among the replicates used for the calibration plate. These data were then fit to the following heuristic_U equation and the coefficients determined by the linear least squares method.

$$U = \frac{\begin{matrix}a + b \times [ probe ] + c \times LN [ primer ] + \\ d \times LN ( amplicon length )\end{matrix}}{DS \times e} \qquad ( Eqn . 80 A )$$

Where in the preferred embodiment the coefficients are:

a=4.28146E17, b=2.10815E23, c=1.89549E16, d=1.29155E16, and e=1.525868 if MGB is present and 1 otherwise, and DS=2 if the target is double stranded and DS=1 if the target is single stranded. This method predicts U for the 51 TaqMan assays with an average deviation of 29% and the largest deviation is 92%. In alternative embodiments, the a, b, c, d, and e coefficients have values within a factor of 3 of those given above.

$$U = \frac{\begin{matrix}a + b \times [ probe ] + c \times LN [ primer ] + \\ d \times LN ( amplicon length )\end{matrix}}{DS \times e \times pdK} \qquad ( Eqn . 80 B )$$

Where the coefficients are:

a=2.16579E17, b=−6.28527E22, c=1.06329E16, d=2.44001E15, and e=1.525868 if MGB is present and 1 otherwise. This method predicts U for the 51 TaqMan assays with an average deviation of 40%. In alternative embodiments, the a, b, c, d, and e coefficients have values within a factor of 3 of those given above.

Alternative embodiments for estimation of U involve SQRT[primer], and adding a term for the pdK, adding a term for LN(pdK), multiplying by pdK, or dividing by pdK. However, none of those alternative functional forms performs as well as the above equation.

Other variables that may affect U are amplicon length (or more correctly the amplicon sequence), primer lengths (or more correctly the primer sequences), [NTPs], [Mg], and enzyme concentration, and perhaps other effects that influence the position of the inflection point. Whichever reagent is limiting in the reaction will have the largest influence on U. In principle the [Mg] could also be limiting since the pyrophosphate generated in the reaction strongly complexes with Mg²⁺ and if the Mg is exhausted, then the PCR reaction will stop. Suppose that [NTPs] is limiting, and that the other reagents are in excess. In that case, the NTPs will be used with each cycle depending upon the sequence of the amplicon not including the primer binding site. Since both strands are assumed to be replicated with equal efficiency. Either G+C or A+T will likely occur more in the amplicon sequence than the others and thus that nucleotide would be exhausted the fastest and the efficiency of reaction would begin to decrease. If we presume equal occurrence of all the nucleotides in the reaction, then a very good approximation to the actual sequence base composition is the amplicon length.

Alternative Estimation of U Using the Second Derivative Minimum for the Intrinsic Reference Point:

The U value obtained at the second derivative max is less dependent upon the [primer] and [probe] and also that the pdK plays less of a role because the reaction is still highly efficient at the second derivative max. Thus, an alternative embodiment is to use the U_{SDMax} to get U prediction equations. However, the U_{inflection} has better clustering of D₀ values than the U_{SDMax}. Thus, the U_{inflection} has the best performance for relative quantification, whereas U_{SDMax} has better performance for predicting U for absolute quantification if a calibration plate is not available. Below is described a method to get the best of both worlds.

Recall that the D₀ can be obtained from U, V, and Fⁿ(D₀), where the n is meant to reflect the one of the six different intrinsic reference points:

D₀=Uⁿ×V×Fⁿ(D₀) (Eqn. 81)

Thus, the equations for D₀ using the inflection point and second derivative maximum give the same D₀ value and the equations can thus be combined:

$${D}_{0} = {U}_{reflect} \times V \times \frac{F ( {D}_{0} )}{{F}_{inflect}} = {U}_{SDma x} \times V \times \frac{F ( {D}_{0} )}{{F}_{SDma x}} \qquad ( Eqn . 82 )$$

Upon canceling V and F(D₀) and rearranging we get:

$${U}_{inflect} = {U}_{SDmax} \times \frac{{F}_{inflect}}{{F}_{SDmax}} \qquad ( Eqn . 83 )$$

The inflection point has better relative quantification, while the U_{SDmax} has a smaller absolute error than U_{inflect}. To minimize errors due to noise, it is preferred that the U_{SDmax} should be averaged over all the replicates available.

### U Prediction Method for Dye Based Detection:

U values were experimentally determined for 13 assays with systematically varied [primer], amplicon length, and [SYBR]. The pdK parameter was derived from the fitting of the qPCR curves and averaging them (after removing outliers and not including those wells with zero copies of DNA) among the replicates used for the calibration plate. The data show that for Dye based assays, the U value is independent of [SYBR] using a range of 0.5× to 2× of the manufacturer recommended [SYBR]. The results also show that U for Dye based assays is independent of [primer] and pdK. However, U shows a strong dependence on amplicon length. Previous results (See, e.g., Zipper, H., Brunner, H., Bernhagen, J. and Vitzthum, F. “Investigations on DNA intercalation and surface binding by SYBR Green I, its structure determination and methodological implications” (2004) Nucleic Acids Res. 32, e103.) showed that as the dye-to-base pair ratio (dbpr) decreases, the SYBR switches from groove binding to intercalation and there is a decrease in fluorescence. The dbpr changes as the PCR reaction progresses to higher cycle numbers. These changes in dbpr happen faster for onger sequences than for shorter sequences. As a result of the Dye binding mechanism and length dependent quenching, there is an observed length dependence to the U value for SYBR dye based detection (and similar length dependence of U is observed for SYTO-13 and EvaGreen dyes that are alternatives to SYBR). Simultaneously, High concentrations of SYBR are known to inhibit PCR, whereas SYTO-13 inhibits PCR to a lesser extent than SYBR.

The 13 experimental U values were then fit to the following length-dependent heuristic_U equations for Dyes:

$$\begin{matrix}U = \frac{a \times {( amplicon length )}^{b}}{DS} & ( Eqn . 80 C ) \\ U = \frac{c + d \times L N ( amplicon length )}{DS} & ( Eqn . 80 D )\end{matrix}$$

where the coefficients are: a=2.89608E17, b=−0.453547, c=1.004636E17, d=−1.388986E16, and DS=2 if the target is double stranded and DS=1 if the target is single stranded. Eqns. 80C and 80D predict the U values for 13 Dye-based assays within 8.6% and 9.6% average deviation, respectively. In alternative embodiments, the a, b, c, and d coefficients have values within a factor of 3 of those given above.

### IV.d Calibration Plate Methods

For best results with qPCR copy counting, it is necessary to obtain an accurate value of U, which is simply the reciprocal of the intrinsically referenced fluorescence for a single copy of DNA divided by the volume. This value of U is assay specific and depends upon the [primers], [probe], [NTPs], amplicon sequence (or length), and [enzyme]. The fluorescence from a single copy can be most accurately determined experimentally by a simple one-time and assay-specific calibration. The calibration consists of performing one plate of measurements on a sample diluted to an average of approximately 0.5 to 3.5 copies per well (the necessary dilution can be accurately determined by performing a preliminary single qPCR reaction). For a 96 well plate, this procedure provides calibration for absolute quantification to about 10% accuracy (and about 5% accuracy for a 384 well plate). Note that this calibration requires no standards. One calibration applies to all instruments and samples. As long as one does not change primer design, primer concentration, or master mix, there is no need to repeat the assay calibration.

### Example of Experimentally Determining a U Calibration for a Given Assay:

Step 0: User provides whole plate (i.e. at least 96 wells) of raw data for a sample that has been diluted to approximately 1 molecule per well on average (in fact, about 1.5 molecules per well is optimal for minimum error). This can be off by up to a factor of 3 (i.e. mean >0.3 and <3.0) for 96 wells (or factor of 4 for 384 wells) without compromising the calibration.

Step 1: Count the number of wells that have zero copies of DNA set Z equal to that number. Determine the number of wells with greater than zero copies of DNA. This is called the “Positive Count”=PC=N−Z, where N=number of replicates.

Step 2: Compute Poisson Mean (Eqn. 44) and the Poisson error (Eqn. 47) (described in Dube, S., Qin, J., and Ramakrishnan, R. (2008) Plos ONE 3(8) e2876). If the number of zeros is fewer than 6, then go to step 2A below. Note that these methods work best for 384 wells or more. For 96 wells, the method works but the error limits are larger.

The Poisson mean copy number per well, M, is estimated using Z and PC:

$$M = - L N ( 1 - \frac{PC}{N} ) \qquad ( Eqn . 44 )$$

If there are no zeros (i.e. PC=N), then set PC=N−1. In addition, the mean copy count will be improved in step 4. The Poisson distribution is not symmetric (particularly if N and PC are both small) and thus we compute the lower standard deviation and the upper standard deviation of the Poisson Mean:

$$\begin{matrix}Lower\_SD = M + L N ( 1 - \frac{PC}{N} + \sqrt{\frac{PC}{{N}^{2}} \times ( 1 - \frac{PC}{N} )} ) & ( Eqn . 45 ) \\ Upper\_SD = - L N ( 1 - \frac{PC}{N} - \sqrt{\frac{PC}{{N}^{2}} \times ( 1 - \frac{PC}{N} )} ) - M & ( Eqn . 46 ) \\ Average\_SD = \frac{Lower\_SD + Uppper\_SD}{2} & ( Eqn . 47 )\end{matrix}$$

Compute the Average_SD given in Eqn. 47. If the number of zeros is fewer than 6, then the Poisson error is unreliable. For 96 wells the minimum error is 12.9% (for 21 zeros). For 384 wells the minimum error is 6.4% (for 79 zeros). Nonetheless, the copy counting method given below is more accurate than the Poisson method given here.

Step 2A: Alternative to Poisson Mean. In the event that the number of zeros is fewer than 6, an alternative method is used to compute the preliminary mean copy number (this preliminary value will be optimized during the minimization of sum_chi_Squared in step 5). Fitting data with a mean copy number greater than 4 is generally not recommended. The estimated mean can be obtained from the following equations (use results from steps 3A and 3B):

$${CV}_{Total} = \frac{{sigma\_F}^{′} ( {D}_{0} )}{{mean\_F}^{′} ( {D}_{0} )} \\ {CV}_{Poisson} = \sqrt{{CV}_{Total}^{2} - {CV}_{Expt}^{2}} \\ M = \frac{1}{{CV}_{Poisson}^{2}} ( 49 , related to Eqn . 60 ) \qquad ( Eqn . 48 )$$

This may be called the “standard deviation method for computing the mean”. Note that CV_expt should be set to 0.03 to correspond to typical user pipetting error. If M is greater than 4, then the results from Eqn. 60 are not reliable because the CVₚₒᵢₛₛₒₙ is too small. Another alternative is to compute M=heuristic_U*V*mean_F′(D₀), which is accurate within about 30% error. The heuristic_U is computed from the parameters [probe], [primer], amplicon length, pdK, MGB, and double strand using Eqn. 80, 80B, or 80C.

Step 2C: If the user provides the mean copy number (e.g. measured from digital PCR), then F₁ is computed using Eqn. 50:

$${F}_{1} = \frac{\overline{F}}{M} \qquad ( Eqn . 50 )$$

No optimization is performed. The calibration error is approximated by:

$$Calibration\_Error = \frac{{sigma\_F}^{′} ( {D}_{0} )}{{mean\_F}^{′} ( {D}_{0} ) \times \sqrt{N}} \qquad ( Eqn . 51 )$$

Step 3: The “no boundaries method” for calculating U. In this method, for a Poisson distribution the mean copy count, M (from Eqn. 44), is given by the average of all the wells, including the zeros. The mean fluorescence, F-bar, is computed by directly averaging all of the F′(D₀) values for the whole replicate set (including the zeros). A preliminary F₁ is then computed using Eqn. 50.

$$M = \frac{\overline{F}}{{F}_{1}} \\ {F}_{1} = \frac{\overline{F}}{M} \qquad ( Eqn . 52 )$$

The F₁ obtained at this point is approximate for two reasons: A. the mean from the Poisson distribution (Eqn. 44) or the mean from the standard deviation of fluorescence (Eqn. 49) are approximate and B. the F-bar is approximate because of the effect of delayed onset. Thus, there is a need to obtain a refined value of F₁ by getting a better estimate of the M and F-bar. Fluorescence values from wells that have delayed onset may not be correct. For example, a qPCR well that appears to exhibit a F′(D₀)=0.5* F₁ is providing evidence that in fact it is a single molecule with a delayed onset of 1 cycle. To correct for this, we identify all the wells that have copy number greater than 0 but less than 0.7, and replace those fluorescence values with the current F₁ and then recompute a refined F-bar. The refined F-bar is in turn used to compute a refined copy number for all wells (Eqn 53). The refined copy counts can then be averaged (including the zeros) to obtain a refined mean. Similarly, the wells with copy numbers of 1.3 to 1.7 or 2.3 to 2.7 also involve delayed onset and are replaced with 2*F₁ and 3*F₁, respectively, and then a further refined F-bar is computed. Such refinement is repeated until a self-consistent solution for F₁ is obtained.

### Details for Implementing the “No Boundaries Method”:

Pseudocode for implementing the “no boundaries method”:

```
    //Store the N values of F′(D₀) in an array F[i]
        Double SUM=0, CC=0;
        For(i=1;i<=N;i++)
        {
            CC= F[i]/F1; //temporary copy count
            if(CC > 0 && CC<0.7) //this is checking if 0<CC<0.7
            {
            SUM += F1; //this corrects all the F[i] values that are due to delayed
onset of PCR
        }
        else SUM += F[i];
    }
    F1 = SUM/(N*M); //division by N to get mean_F′(D0), division by M gives the F1
    Note: this new F₁ is more accurate than the original F₁. The new F₁ can be further
    improved (by about 1 percent) by running the pseudocode again, but using the new
    F₁ value to compute the temporary copycount and also in the SUM.
```

A. Subroutine (pass in F₁ and the F′(D₀) values, pass out the CC values and the M). Use the new F₁ from step E to convert all the F′(D₀) values to CC using Eqn. 53. Use our rounding method (if (0<CC<0.7) then CC=1, else perform normal rounding). Using the rounded CC values, compute a new mean of the copy counts (including the zeros), call this M1 (step 4G shows an alternative to compute M1).
B. Subroutine. Set up an array, O[K], where K is an index corresponding to the copy count and O[K] stores the number of wells that have copy count, K. In step 4, these O(K) values will be compared to the Poisson distribution expected number of wells that have copy count K, E(K). An alternative to compute M1 (“max K” in this equation is the largest K value observed on the plate):

$$Average\_Copy \_Number = M 1 = \frac{{\sum }_{K = 0}^{max K} ( K \times O ( K ) )}{N} \qquad ( Eqn . 54 )$$

Step 4: Refinement of F₁ by minimizing the sum of chi squared. The basis for this method is to improve the quality of F₁ by utilizing the mean, the width, and the asymmetry of the distribution. In this step, we are no longer depending on the positive count, which itself has some error. In principle, this refinement method even works if no zeros are observed, as long as the Poisson error is the largest contribution to the error (which occurs for mean copy counts less than 10).

This refinement is performed as follows:
A. Set up a subroutine to compute the expected Poisson distribution.

Definitions:

K=number of copies of DNA in a well (integer)

N=number of wells

M=mean copy number per well (the Ml calculated in step 3, Eqn. 54)

P(K)=probability of observing K in a given well (double precision)

E(K)=expected number of wells that contain K copies (double precision)

$$\begin{matrix}P ( K ) = \frac{{M}^{K} \times {e}^{- M}}{K !} & ( Eqn . 55 ) \\ E ( K ) = N \times P ( K ) & ( Eqn . 56 )\end{matrix}$$

Compute E(K) for K=0, 1, 2, 3, . . . , max_K+3. The “max_K” is the largest K value on the plate. Store these E(K) values in an array. This max_K value is needed in step 4B.
B. Combining low population bins.
   Problem: One of the dangers of the chi-squared method is that low population bins (i.e. K values) can make a disproportionate contribution to the chi-squared-P (step 5). For example, suppose that a bin with K=8 is expected to have an occurrence of 0.01, but by chance a K=8 occurs once, then its contribution to the sum chi-squared is (1−0.01)̂2/0.01=99, which is a huge (and non-physical) contribution that would result in a Chi_Sq_P<<0.05 (i.e. declaration as non-Poisson). To deal with this, the statistics community has developed the rule of thumb to combine bins that have a frequency lower than 6. In our case, we are concerned with low counts for either E(K) or O(K) values from step 3G. These can occur at the beginning of the distribution (i.e. low copy number bins) or the end (i.e. high copy number bins).
   Pseudocode is provided for both cases:

```
Combining high copy number bins: Start with the largest K with an E(K) > 0.001
(declare int hi_max_K set equal to that K value). Determine the largest K with an
O(K) >= 1 and call that “Obs_max_K”. If Obs_max_K > hi_max_K then set
hi_max_K = Obs_max_K. Then combine the bins as follows:
double sum_hi_E=0, sum_hi_O=0; //expected and observed sums of combined bins
for(i=hi_max_K; sum_hi_E<6;i−−){ //&& sum_hi_O<6
  sum_hi_E += E(i);
  sum_hi_O += O(i);
}
int hi_min_K = i; //so the bins that are combined range from hi_min_K to
hi_max_K, inclusive. Be sure not to decrement i too much here.
chi-sq contribution = (sum_hi_E − sum_hi_O)\hat{ }2/sum_hi_E
```

```
Combining low copy number bins for case where mean is large: Combine the low K
bins as follows:
double sum_lo_E=0, sum_lo_O=0; //expected and observed sums of combined bins
for(i=0; sum_lo_E<6 ;i++){ //&& sum_lo_O<6
  sum_lo_E += E(i);
  sum_lo_O += O(i);
} //note that if the K=0 bin already has 6 or more members, then i=0 at this point
int lo_max_K = i; //so the bins that are combined range from 0 to lo_max_K,
inclusive. Be sure not to increment i too much here.
chi-sq contribution = (sum_lo_E − sum_lo_O)\hat{ }2/sum_lo_E
```

C. Set up a subroutine to compute the sum of chi squared (the Sum goes to max_K+3 because the Poisson distribution may predict significant numbers of counts for larger K values than observed experimentally and we want the optimization algorithm to take that into account):

$$Sum\_chi \_squared = {\sum }_{K = 0}^{max K + 3} \frac{{( O ( K ) - E ( K ) )}^{2}}{E ( K )} \qquad ( Eqn . 57 )$$

D. The F₁ from step 3 is likely within 5% of the correct value. Nonetheless, we will search adjusted values of F1 ranging from 0.8*F1 to 1.2*F1 in increments of 0.005 (e.g. try 0.8*F1, then try 0.805*F1, then 0.810*F1, etc.). A total of 81 different trials are tested. Alternative embodiments search a wider range and with better resolution than 0.005 (though the minimum is NOT a smooth function—see Appendix FIG. 3). For each of these adjusted F₁ values, compute the CC, M1, and O(k) as in step 3. Also compute the E(K) and the sum_chi_squared. Keep the F1 that results in the minimum sum_chi_squared.
E. Compute U:

$$U = \frac{1}{V \times {F}_{1}} \qquad ( Eqn . 58 )$$

Analysis (using Poisson sampling analysis) of the Error in U from the copy counting method shows that the error is approximately (if the mean is <1.5):

$$Sigma\_U = \frac{U}{\sqrt{N \times M}} \qquad ( Eqn . 59 )$$

Larger means result in higher Sigma_U, which is best approximated using Eqn. 47. Below is the equation for calculating the calibration error (for M<1.5):

$$Calibration\_Error = \frac{1}{\sqrt{N \times M}} \qquad ( Eqn . 60 )$$

Step 5: Compute the chi-squared P. The closer chi_squared_P is to 1 the better (i.e. it can be concluded that the observed distribution agrees with the predicted Poisson distribution). If Chi-squared-P is <0.05, that indicates that the observed distribution is not Poisson. In this event, it is likely that the assay itself needs to be re-designed or that the calibration plate needs to be re-run to determine whether there was an error in the initial calibration, e.g., a mistake was made in setting up the plate.

The Chi-squared P, is computed in Excel with the following equation:

Chi_squared_P=1−CHISQ.Dist(Sum_Chi_squared, max, true)

The first argument is the sum of chi squared from step 4B. The second argument is the degrees of freedom=max (it is the number of bins −1 and the number of bins is max +1 because the zeros bin is also included), the third argument indicates that we are using the “Cumulative Chi Squared distribution”. An example value is Chi_squared_P=0.987 for sum chi squared=0.627 and dof=5.

Step 6: Cluster Method for Calibration of U. In certain cases (e.g. when the number of wells in the calibration plate is <300 or if the delayed onset fraction is >0.4) it is preferable to determine the U for a given plate by analyzing the clusters of F′(D₀). Figure XA shows a plot of the ranked F′(D₀) values for the human EGFR gene. This calibration plate exhibits a delayed onset fraction of ˜24%. The basic idea of the cluster method is to identify the cluster of F′(D₀) values that corresponds to a single molecule at cycle zero (recall that the calibration plates are set up using a mean copy number of approximately 1.5 copies of DNA per well so that it is expected that the largest cluster corresponds to 1 molecule or 2 molecules of target DNA). The average of the values in that cluster corresponds to F₁.

Here is pseudocode to get the correct cluster corresponding to F₁:

//Sort the F′(D0) from lowest to highest

| | |
|---|---|
| int counter[i] = 0; | //These are arrays of dimension N+1 |
| double average[i]=0; | //where N is the number of wells, the zeroth element is not used |

```
//first compute all clusters
for (i=1; i<=N,i++) // i is an index for each well and its corresponding F(i)
{ //compute the number of wells that have F′(D0) within +/− 10% of guessed F1
      for (j=1; j<=N,j++)
      {
            if( F(j) > 0 && F(j) > F(i) − F1*0.1 && F(j) < F(i) + F1*0.1)
            {
                  counter[i]++;
                  //average[i] += F(j); //this gives the sum of all the F(j) in the cluster
            //this might not be needed
            }
            if(F(j) > F(i) + F1*0.1) break; //no need to keep searching because the F[i]
      are already sorted
      }
      //average[i] /= counter[i]; //this gives the average F for all the F(j) in the cluster
      //this is not correct. Not the whole cluster but the middle of the cluster.
}
//find the cluster with the maximum value
int max=0;
int max_i=0;
for (i=1; i<=N,i++)
{
      if(max < cluster[i]) //in the event that a cluster with equal value is found keep the
one with the smaller value of max_i
      {
            max = cluster[i]; //this store the size of the largest cluster
            max_i = i;
      }
}
//get the average of the sub cluster (i.e. the F values that have clusters within 2 of the max
cluster)
int counter = 0; //3% or 2 whichever is larger
double average_F1 = 0;
for(i=max_i; cluster[i] > cluster[max_i] − 3 ;i−−)
{
      average_F1 += F[i];
      counter++;
}
for(i=max_i + 1; cluster[i] > cluster[max_i] − 3 ;i++)
{
      average_F1 += F[i];
      counter++;
}
average_F1 /= counter; //this gives the actual average for the sub cluster
double ratio = 0;
ratio = average_F1 / F1;
//Check if this is for F1 or F2
if (ratio >= 0.7 && ratio < 1.3) final_F1 = average[max_i]; //this is the usual case
else if (ratio >= 1.3 && ratio < 1.7) final_F1 = average[max_i] / 1.5; //this should very
rarely happen
else if (ratio >= 1.7 && ratio < 2.3) final_F1 = average[max_i] / 2.0; //this could happen if
the mean copy number is higher than 2
else use no boundaries method
Compute Cluster_fraction = max / PC; //where PC is the positive count
if(Cluster_fraction < 0.17) then use old method to get U (i.e. with chi squared optimization)
//End Pseudocode for Cluster Method
```

Step 7: Compute the raw_CopyCount (which is a double) for each well.

$$CopyCount = \frac{{F}^{′} ( {D}_{0} )}{{F}_{1}} \qquad ( Eqn . 53 )$$

where F₁ in this equation is the Final_F1 computed by the cluster method.

Step 8: Special rounding of raw_CopyCount to integer. This corrects the raw copycounts and ensures that all the wells that are delayed onset are rounding to the proper higher integer. This accounts for the fact that there is some experimental error in the F′(D₀) values (i.e. a D₀=0.37 may actually be a D₀=0.5 case and it should be rounded up to D₀=1).

Pseudocode:

```
for(i=1; k<=N;i++)
{
    if(raw_CopyCount[i] > 0 && raw_CopyCount[i] < 1.0)
        integer_CopyCount[i] = 1;
    else
        integer_CopyCount[i] = round (raw_CopyCount[i] + 0.15);
}
```

Step 9: Computing the “Delayed Onset Fraction”, DOF. Certain qPCR assays demonstrate a high fraction of wells that exhibit delayed onset (i.e. where the first cycle of PCR does not amplify the target. This can occur is cases where the target DNA is not fully denatured during the denaturation step of a PCR cycle. This effect is observed most prominently for double-stranded human genomic DNA or circular genomic DNA. Typically the amount of delayed onset for double-stranded genomic DNA ranges from 15 to 30% for well-designed assays and higher for poorly designed assays. Other samples such as single-stranded cDNA, typically exhibit low amounts of delayed onset (typically 0-15%). Such delayed onset needs to be accounted for to properly calibrate U for assays that have a high DOF. Experimentally, the amount of delayed onset can be reduced by increasing the denaturation temperature or time for the first 3 cycles of PCR, increasing the primer concentration, or redesigning the primers.

The computation of the delayed onset occurs in two steps: First the amount of delayed onset that is apparent in the wells that have D₀ of approximately 0.5 and approximately 1.5 are accounted for. Second, the effect of double delayed onset (i.e. when two molecules are simultaneously delayed) is accounted for. First the raw D0 values are computed as given in step 7. The F′(D₀) are then grouped into bins:

Bin_0.5=D₀ values of 0.2 to 0.7

Bin_1=D₀ values of 0.7 to 1.3

Bin_1.5=D₀ values of 1.3 to 1.7

Bin_2=D₀ values of 1.7 to 2.3

Bin_2.5=D₀ values of 2.3 to 2.7

Bin_3=D₀ values of 2.7 to 2.3

Each bin simply contains the count of the number of wells that have D0 within the range given. For example, if 25 wells have a raw D0 between 0.7 and 1.3, then the Bin_1=25.

Preliminary Calculation of DOF

$$Preliminary\_DOF = \frac{Bin\_ 0.5 + Bin\_ 1.5}{Bin\_ 0.5 + Bin\_ 1.0 + 2 * Bin\_ 1.5 + 2 * Bin\_ 2} \qquad ( Eqn . 84 )$$

For calibration plates, delayed onset can also occur where two molecules in the same well are delayed. This has the effect of making K=2 wells look like K=1 and makes K=3 look like K=2, and K=4 like K=3. A refined value for the delayed onset that accounts for single and double delayed onset is given as follows:

$$D O F = \frac{\begin{matrix}Bin\_ 0.5 + ( 1 + 2 * Preliminary\_DOF ) * Bin\_ 1.5 + \\ ( 1 + 2 * Preliminary\_DOF ) * Bin\_ 2.5\end{matrix}}{\begin{matrix}Bin\_ 0.5 + Bin\_ 1.0 + 2 * Bin\_ 1.5 + \\ 2 * Bin\_ 2 + 3 * Bin\_ 2.5 + 3 * Bin\_ 3\end{matrix}} \qquad ( Eqn . 85 )$$

The idea of the above equation is that if a double delay occurred, then the number of ones that would result from a two being double delayed would be equal to the number of wells with “1.5 copies” times the fraction of delayed onset that occurs. In the numerator, the “Delayed_onset” is multiplied by 2 because such double delay involves two molecules being delayed. The above equation can be generalized to account for triple or higher delayed onset (i.e. three or more molecules simultaneously being delayed), but such events are rare and thus can be neglected to a good approximation.

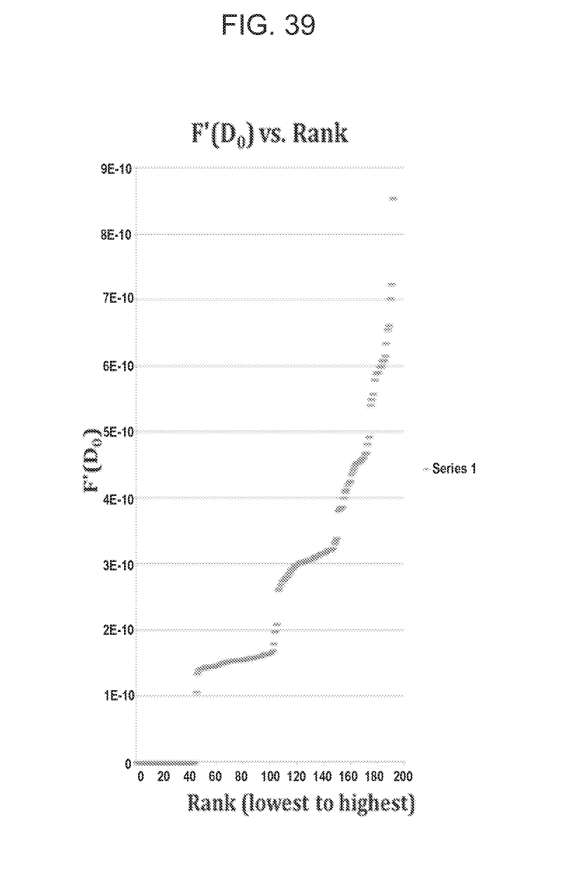

FIG. 39 shows a plot of F′(D₀) vs. rank number for human ACTB cDNA, which has little delayed onset (Delayed Onset Fraction ˜2%). F₁ in this example is approximately 1.5E-10. Note that there are few wells with D₀=0.5, 1.5, or 2.5.

### V. COUNTING COPIES OF DNA

The procedure for counting copies of DNA builds upon the MAK2 model. Intrinsic referencing can then be applied.

The two parameters of MAK2 are the DNA copy number at cycle zero, D₀, and a parameter, k, which accounts for the cycle-by-cycle change in amplification efficiency. MAK2 was previously limited to only providing relative quantification of the same target in different samples. DNA Software, has added many enhancements to MAK2 to create qPCR CopyCount, which allows for completely automated curve fitting for all major PCR instruments, provides error analysis, allows for relative quantification of different targets, allows for multiplexed detection and analysis, and greatly improves the reliability of the method. Most importantly, the present invention realizes how to extend the capabilities of qPCR CopyCount to include the method of cPCR so that now absolute quantification is possible for all qPCR reactions without using any standards. This breakthrough in understanding the mechanism of qPCR will have significant implications for DNA-based diagnostics.

V.a. Fitting the qPCR curve: The MAK2 model (see Boggy and Wolf, 2010) fits the shape of the qPCR curve to determine the fluorescence from DNA at cycle zero, F(D₀).

As noted above, background fluorescence that is independent of the DNA accumulation is adjusted in MAK2-fitting using Equation 3,

Fₙ=F(Dₙ)+Fₘ·n+F_{b} (3)

where F(Dₙ) is the MAK2-predicted fluorescence due to dsDNA at cycle n, F_{b} (also termed F_{background}) represents constant background fluorescence intercept, Fₘ is the background slope, and Fₙ is the predicted fluorescence at cycle n, the variable used for fitting qPCR fluorescence data. At cycle zero this reduces to:

F₀=F(D₀)+F_{b} (63)

Thus, F(D₀) is not the total fluorescence at cycle zero, since the background fluorescence F_{b} must be considered.

In most instances, the magnitude of F_{b} is much larger than that of F(D₀), which is typically completely lost in the noise during early PCR cycles. FIG. 1 shows a graphic representation of raw qPCR data (diamonds) with the fit (boxes) from a qPCR copy count determination for a target having exactly one (1) copy at cycle zero. For this dataset, the copy counting analysis gives: F(D₀)=3.876E-6 (arbitrary fluorescence units, AFU) and Fb(background)=7739 AFU. The RMS noise for the first 10 cycles is 31.5 AFU (0.4%). The signal of interest, F(D₀), is more than 8 million times smaller than the RMS noise. Nonetheless, the qPCR copy counting method of the technology of the present invention provides an accurate determination of F(D₀). As discussed above, F(D₀) is proportional to the absolute number of copies of DNA in the original sample (D₀).

It is a key property of the MAK2 model that it uses a mechanistic model for amplification to fit the data points in the bend in the PCR curve where the signal-to-noise ratio is excellent, to deduce the amount of DNA at cycle zero.

As discussed above, F(D₀) is proportional to the absolute number of copies of DNA in the original sample, called D₀, according to the following equation:

D₀=c·F(D₀) (2)

where c is a proportionality constant. The value of constant c is different for each instrument (and even each well on the same plate), and is dependent upon many factors, such as the extinction coefficient, fluorescence quantum yield, temperature, instrument geometry, filters used, master mix composition ([Mg], [NTPs], [Enzyme], etc.), primer concentration, amplicon sequence, and many more effects, and is generally unknown. Without determining a value of c, relative quantification of DNA can be accomplished, as discussed above. When absolute quantitation is desired, the effect of c is generally determined empirically (e.g., using the C_{q} standard curve method or digital PCR).

In developing methods of the present technology, a method of determining the value of c has been developed, such that an absolute count of DNA copy number may be obtained without the use, e.g., of empirical methods such as the C_{q} and digital PCR methods discussed above. Using the technology of the invention, the value of c can be inferred in a fashion that is independent of the instrument, fluorophore type, and accounts for many assay dependent variables. As a result, with use of the technology, every qPCR is an absolute qPCR and, for most applications there is no need for separate calibration by digital PCR or C_{q} standard curve.

Equation 2 can be reformulated so that it is independent of the instrument. Equation 2 can be written for two different qPCR reactions as follows:

$$\begin{matrix}{D}_{0}^{A} = {c}^{A} \cdot F ( {D}_{0}^{A} ) & ( 64 ) \\ {D}_{0}^{B} = {c}^{B} \cdot F ( {D}_{0}^{B} ) & ( 65 ) \\ \frac{{D}_{0}^{A}}{{D}_{0}^{B}} = \frac{{c}^{A} \cdot F ( {D}_{0}^{A} )}{{c}^{B} \cdot F ( {D}_{0}^{B} )} & ( 66 )\end{matrix}$$

If the two qPCR reactions performed on two separate samples but the assays correspond to the same target with the same reagents (i.e. same master mix and primers) using the same instrument to measure the two qPCRs, then the constants c^{A} and c^{B} are the same and ratio of the two equations (Eqn. 66) shows that the ratio of the DNA copies, D₀, equals the ratio of the F(D₀). Thus, the ratio of F(D₀) for different samples is useful for relative quantification and this is a significant application of the MAK2 method described in the U.S. Patent Appl. Ser. No. 61/528,758 and Ser. No. 13/598,599. The basic limitation of equation 66 is that the constants c^{A} and c^{B} are unequal for different targets (i.e. different primers result in different amplicons), different fluorophores, different master mix, and different instruments.

The goal of copy counting PCR (cPCR) is to determine the fluorescence from a single copy of DNA and to use that value to determine the number of copies of DNA in an unknown from its F(D₀), as shown in Equation 67:

$$DNA Copy Number at cycle zero = {D}_{0} = \frac{F ( {D}_{0} = unknown )}{F ( {D}_{0} = 1 copy )} \qquad ( 67 )$$

where F(D₀=unknown) is the fluorescence from DNA at cycle zero for a sample with an unknown number of DNA copies.

Comparison of Equations 2 and 67 reveals that that the “c” in equation 2 is simply 1/F(D₀=1 copy).

Equation 67 can also be applied generally to the fluorescence from DNA at cycle n of PCR, F(Dₙ):

$$DNA Copy Number at cycle n = {D}_{n} = \frac{F ( {D}_{n} = unknown )}{F ( {D}_{0} = 1 copy )} \qquad ( 68 )$$

Equation 68 can be used to compute the number of copies of DNA present at any cycle n of PCR, Dₙ. As the PCR reaction progresses, the number of copies of DNA increases and equation 68 is used to count the number of copies of DNA at each cycle of PCR.

There are several challenges with actually applying equations 67 and 68. First, most PCR curve fitting methods do not provide the fluorescence from DNA alone at cycle zero. This problem is obviated by using the MAK2 method discussed above. Second, there is also the problem that F(Dₙ) and F(D₀=1 copy) generally need to be acquired on the same “instrument” and that is often difficult to accurately reproduce because even different wells on the same machine have different illumination intensity, different materials in the optical path, and different detection geometry, filtering, and detection efficiency. To remove this problem, the copy counting method of the technology applies the intrinsic referencing methods described above. Thus, Equations 67 and 68 can be recast in terms of F′ quantities as follows:

$$DNA Copy Number at cycle zero = {D}_{0} = \frac{{F}^{′} ( {D}_{0} = unknown )}{{F}^{′} ( {D}_{0} = 1 copy )} \qquad ( 69 )$$

The F′ quantities shown in the equations above are both instrument independent.

Methods of determining the value for F′(D₀=1 copy), e.g., by empirical calibration of each assay design, by general calibration, and by theoretical calibration, are discussed hereinabove.

### VI. EXPERIMENTAL EXAMPLES

The following examples are provided in order to demonstrate and further illustrate certain preferred embodiments and aspects of the present invention and are not to be construed as limiting the scope thereof.

### Example 1

### Exemplary Fitting Using MAK2

Embodiments of the technology use the MAK2 fitting method of Boggy and Woolf One example of application of MAK2 fitting is provided below. See also Boggy G J and Woolf P J, PLoS One 5(8) e12355 (2010), U.S. Patent Appl. Ser. No. 61/528,758, and Ser. No. 13/598,599.

Details and examples of code, implementing MAK2 are shown below, which have been implemented in one embodiment to carry out the processes outlined in the flow chart of FIG. 1 are shown below.

The sum of squared residuals is used as the cost function for optimization. Each iteration of optimization tests values for parameters D₀, k, and F_{b} by performing a simulation of MAK2 with these values and calculating the associated cost function value. Parameter values resulting in the minimum cost function value found in 5000 iterations of Nelder-Mead optimization, e.g., are considered a correct parameter set.

The data included for optimization may be truncated to the cycle with the maximum slope increase relative to the previous cycle. Values for slope (equivalent to the first derivative with respect to cycle) are obtained by subtracting fluorescence at the previous cycle from the fluorescence for the current cycle. Values for slope increase (equivalent to the second derivative with respect to cycle) are obtained by subtracting the previous cycle's slope value from the current cycle's slope value.

The parameters in the MAK2 model are fit using custom developed Mathematica code. First, dataCTfunc is a function to define the data to be fitted. The variable ‘data’ is the name of the variable to be evaluated. The variable ‘frac’ defines the fraction of the maximum signal threshold used for excluding data in the set. Examples of data to fit are described in by Boggy and Woolf, supra.

```
dataCTfunc[data_] t=
  {
    b = Abs[data[[1, 2]]] + 0.0001;
    datadiff = { };
    For[i = 1, i < Length[data], i ++;
      diff = data[[i, 2]] − data[[i − 1, 2]]; datadiff = Append[datadiff, diff]];
    datadiffdiff = { };
    avddiffdiff = { };
    For[i = 1, i < Length[data] − 1, i++; diffdiff = datadiff[[i]] − datadiff[[i − 1]];
      datadiffdiff = Append[datadiffdiff, diffdiff]];
    For[i = 1, i < Length[data] − 2, i++; av = datadiffdiff[[i]] + datadiffdiff[[i − 1]];
      avdiffdiff = Append[avdiffdiff, av]];
    pos = position[avdiffdiff, Max[avdiffdiff]][[1]][[1]];
    CT = position[datadiffdiff, Max[datadiffdiff[[pos ;; pos + 1]]]][[1]][[1]] + 2;
    data[[1 ;; CT]]}[[1]];
```

Next, a cost function is defined using the residual sum of squares. The variable ‘yList’ is the list of DNA values and the variable ‘intensity’ is a list of the corresponding fluorescence intensity values. These lists are populated during each iteration of optimization when a simulation with the current parameter values is performed. The for loop performs this operation. The variable ‘q’ is the current dsDNA value and ‘d’ is the value for the previous cycle. The data used to evaluate the cost function are determined by ‘dataCTfunc’ above.

```
c[x; {_?NumberQ..}] ;= TimeConstrained[Module[{ }, {
      yList = {d0} / . Thread[par -> x];
      intensity = { };
      q = d + k + Log[i + d / k] /. Thread[par → x];
      For[count = 1, count < Length[dataCT] + 1, count++, y1 = q / . d → yList[[count]];
        yList = Append[yList; y1];
        intensity = Append[intensity, (y1 + base) / . Thread[par -> x]]];
      Total[[(#[[2]] − intensity[[#[[1]]]]) \hat{ } 2 & /@dataCT)]}][[1]], 2];
```

The optimization method is defined in the following code block. Nelder-Mead (also known as downhill simplex or amoeba) nonlinear optimization is used (see, e.g., Nelder J A & Mead R. “A simplex method for function minimization”. Computer Journal 7: 308-313 (1965)). The number of loops is initialized with the variable ‘NumberOfLoops’. If the optimization algorithm converges on a local minimum, a new loop will begin and start optimizing from a new point. ‘PertuAmp’ is the variable that affects the window size for an initial parameter value for a new loop.

```
method =
  {{“DifferentialEvolution”, “SearchPoints” → 400, “RandomSeed” → RandomInteger[{1, 100}]},
    {“SimulatedAnnealing”, “PerturbationScale” → 3, “BoltzmannExponent” →
      $Function [ \{ 1 , df , f0 \} , - \frac{df}{{e}^{\frac{1}{10}}} ] , “ RandomSeed ” \to RandomInteger [ \{ 1 , 100 \} ] \} ,$
    {“NelderMead”, “ShrinkRatio” → 0.95\grave{ }, “ContractRatio” → 0.95\grave{ },
      “ReflectRatio” → 1.5\grave{ }, “RandomSeed” → RandomInteger[{1, 100}]},
    {Automatic, “RandomSeed” → RandomInteger[{1, 100}]},
MethodType = 3;
NumberOfLoops = 0;
PertuAmp = 0.9; (*you can fix amplitude of perturbation
  $or make it a decreasing function of the loop number \frac{1}{{10}^{NumberOfLoops}} *)$
```

MAK2 (below) is the main function. The variables ‘data’ and ‘frac’ are passed to ‘dataCTfunc’. The three parameters and corresponding constraints must be defined. An initial parameter set is also defined with ‘current fit’. The maximum number of optimization iterations is set with ‘MaxIter’. No new loops (see below) will be started after this value has been reached. If the current cost function value is lower than any previous value, then the display is updated.

```
MAK2[data_] :=
  {SelectionMove[nb, All, GeneratedCell];
    coutlow = 1;
    dataCT = dataCTfunc[data];
    par = {d0, k, base}; dhigh = .01; dlow = 10\hat{ } (−12);
    constraints = Join[{d0 < dhigh, d0 > dlow,
      k < Max[data[[All, 2]]], k> 0.001, base >−Abs[i.1*b], base < Abs[i.1*b]]];
    (d0 → dlow * 100, k → 1.0, base → b) [[All, 2]] >> “current_fit”;
    MaxIter = 5000;
    ShowStatus[status_] := LinkWrite[$ParentLink,
    SetNotebookStatusLine[FrontEnd EvaluationNotebook[ ], ToString[status]]];
    nb = EvaluationNotebook[ ];
    spar = ToString/@par;
    i = 0;
    NumberOfLoops = 0;
    While[i < MaxIter, NumberOfLoops++;
    curr = ReadList[“current_fit”][[i]];
    v = Flatten[#, 1] & /@Transpose[(par, N[{1 − PertuAmp, 1 + PertuAmp} (#)] & /@Abs[curr]}];
    rt = SessionTime[ ];
    NMinimize[{c[par], constratints}, v, Method → method[[3]],
    MaxIterations → 1000, StepMonitor → (par >> “current_fit”;
      cout = N[Round[c[par] * 10\hat{ }8] / (10\hat{ }8)];
      If[cout ≦ coutlow * 0.99999,
        lowoutRoutine = {Module[{st = “Cu” <> ToString[N[(c[par])]] <> “ ”<>“i=”<>
            ToString[i]<>“ ”<>“tx”<>ToString[Round[SessionTime[ ] − rt]]<>
            “ ”<>“loops=”<>ToString[NumberOfLoops]},
          ShowStatus[st]; NotebookDelete[nb];
          Print[show[(ListPlot[data, PlotLable -> st,
              PlotRange → All], ListLinePlot[intensity, PlotRange → All]}]];
          rt = SessionTime{ };]; print{Thread[spar → par]]; SelectionMove{
          nb, All, GeneratedCell]; coutlow = cout];];
      i++)]];
  };
```

The F(D₀) values obtained are used to generate plots for MAK2 quantification. To generate C_{q} values, first a quantification threshold is chosen that represented about 10% of the maximum signal achieved in a data set (0.1 for our data, 0.05 for rutledge data, and 1 for reps data). Background intensity is determined as described above for determining data to include in MAK2 model-fitting. The C_{q} is calculated as the fractional cycle (linearly interpolated) where (intensity—background intensity) was equal to the quantification threshold.

Code for calculating C_{q} is as follows:

```
CTRoutine[data_, signal_] : =
  {For[i = Position[Abs[0.5 * Max[data[[All, 2]]] − data[[All, 2]]],
        Min[Abs[0.5 * Max[data[[All, 2]]] − data[[All, 2]]]]][[1]][[
      1]], data[[i, 2]] > data[[i − 1, 2]], i−−};
    base = Mean[data[[1 ;; i, 2]]];
    For[j = i + 1, data[[j, 2]] − base <= signal, j++];
    j − 1 + (signal + base − data[[j − 1, 2]]) / (data[[j, 2]] − data[[j − 1,
    2 ]])
    }[[1]];
```

The exponential function used for fitting qPCR data is (Equation 71):

Fₙ=F(D₀)*Eⁿ+F_{b} (71)

where Fₙ is the fluorescence intensity at cycle n, F_{b} is background fluorescence, E is the constant amplification efficiency of the reaction, and F(D₀) is the initial fluorescence.

Data are fit with Equation 71 using nonlinear model-fitting (NonlinearModelFit function) in Mathematica. The data used for fitting is the minimum amount of data (beginning with cycle 1) that results in a nonlinear fit of the data. The D₀ values are used to generate plots for quantification by exponential curve-fitting.

The equation for the five-parameter log-logistic function is (Equation 72):

$${F}_{n} = {F}_{b} + \frac{{F}_{max} - {F}_{b}}{{( 1 + {e}^{q * ( log ( n ) - log ( r ) )} )}^{s}} \qquad ( 72 )$$

where Fₙ, F_{b}, and Fₘᵢₙ are the fluorescence at cycle n, background fluorescence, and maximum fluorescence, respectively; and parameters q, r, and s adjust the shape of the curve. The logistic model is identical to the log-logistic model in Equation 72 except the (log(n)−log(r)) term is replaced by (n−r). Parameter s in Equation 72 accounts for asymmetry in qPCR data and the four-parameter model is a special case of the five-parameter model, where s=1. The first reported sigmoidal model for quantifying qPCR data was a 4-parameter logistic model (Liu W H, Saint D A, “Validation of a quantitative method for real time PCR kinetics”, Biochemical and Biophysical Research Communications 294: 347-353 (2002)). Spiess et al. found that log-logistic models often perform better at data-fitting than logistic models (“Highly accurate sigmoidal fitting of real-time PCR data by introducing a parameter for asymmetry”, BMC Bioinformatics 9: 221 (2008)), so 4- and 5-parameter log-logistic functions may be used in the comparison of quantification methods.

Fitting data with four and five-parameter log-logistic functions is performed in the R package qpcR. The function perbatch is used for batch fitting an entire data set and the value for sig.init2 is used for estimating the initial fluorescence for each run. This estimate is generated by fitting qPCR data with the log-logistic model and then fitting the log-logistic model with the exponential model in Equation 71 to find F(D₀).

### Example 2

### qPCR Copy Counting

The qPCR copy counting method was applied to the quantification of a single housekeeping gene, GAPDH (glyceraldehyde phosphate dehydrogenase). The experiments were performed using a Fluidigm BioMark HD System with the Dynamic Array™ IFC 96×96 chip, each chamber on the chip having a volume of 6.7 nL. The raw quantitative amplification data set was provided by Gang Sun, Fluidigm Corporation.

A series of 72 replicates of 15 dilutions (3-fold dilution each) were performed (total of 1080 qPCR reactions). Raw qPCR TaqMan fluorescence (FAM dye) vs. cycle data (raw data provided by Fluidigm Corp.), along with the passive ROX fluorescence data for the 1080 qPCR reactions were analyzed. The dilution proportion and sample layout were not input in the fitting program. The qPCR copy counting method was then used to fit all of the data, and the resulting quantitative relative F(D₀), which were graphed against the known concentrations (FIGS. 6A and 6B). The results show that the F(D₀) values from qPCR copy counting method are comparable in accuracy to results obtainable with a Cq standard curve (although no standard was used and no Cq standard curve was generated).

The standard error in D₀ in the nine highest dilutions is 10%. Larger errors in the six lowest concentrations are larger due to low sampling of the Poisson distribution. The three lowest dilutions (i.e. experimental relative concentrations) have 12, 26, and 62 replicates that have D₀>0, while 60, 46, and 10 replicates have D₀=0, for the respective dilutions.

The same raw data set was further analyzed by performing replicate averaging and using the copy counting (cPCR) principles to determine absolute copy number quantification (FIG. 7). There is excellent linearity in the trend line down to a 1 million fold dilution (0.000001 on the X-axis). The last three points fall off the line due to the low copy number resulting in some of the replicates having F(D₀)=0.

However, at low mean copy number some of the wells have F(D₀)=0, which corresponds to zero molecules of DNA. Wells with zero copies of DNA are perfectly valid data points and should be included in the averaging process. Upon inclusion of the F(D₀)=0 replicates into the average (i.e. by dividing by the total number of replicates, 72, instead of dividing by the number of replicates with F(D₀)>0), the plot shown in FIG. 8 was obtained. The results show outstanding linearity over a 4.5 million fold range in concentration.

### Counting Copies of DNA for Absolute Quantification

The results shown in FIG. 8 demonstrate that the fitting method is a reliable method for distinguishing those wells that contain sample DNA (i.e. in which F(D₀)>0), from those that do not (i.e. F(D₀)=0). Based upon that observation, Poisson statistical analysis as described by (Dube, Qin et al. 2008) was applied. Specifically, the data from the three lowest dilutions in FIG. 7 were analyzed and an error-weighted average was determined (FIG. 9).

FIG. 9 shows the results from application of the Poisson statistics to the prior data set. The results indicate that the copy counting method can reliably deduce absolute quantification of copy numbers if sufficient replicates are available for each dilution. The standard deviation in the absolute quantification is 11.2%.

An understanding of the difference between accuracy and precision is critical to interpreting the results from qPCR CopyCount. The three most important metrics to understand are σ_{Relative}, σ_{Calibration}, and σ_{Absolute}. Respectively, these three metrics correspond to the precision (i.e. for relative quantification), the systematic error, and accuracy (i.e. for absolute quantification) of the DNA copy count.

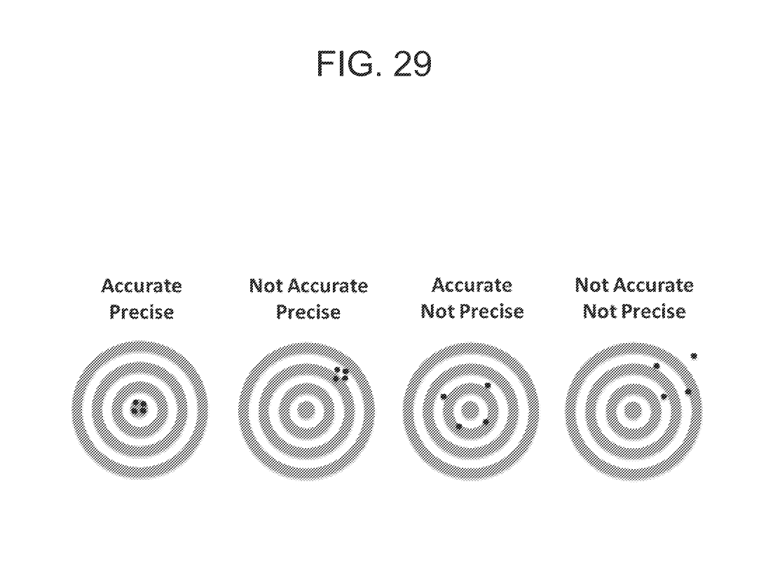

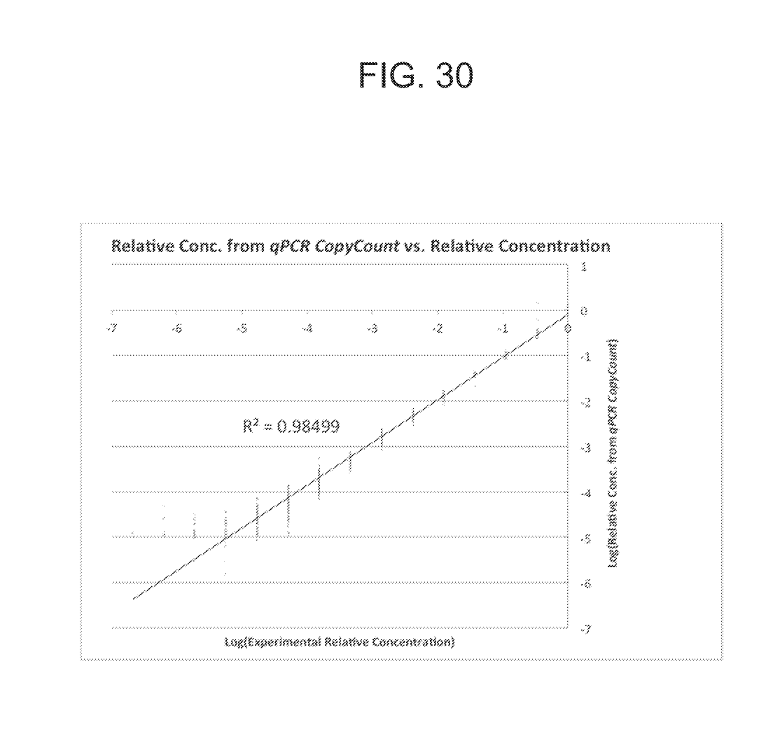

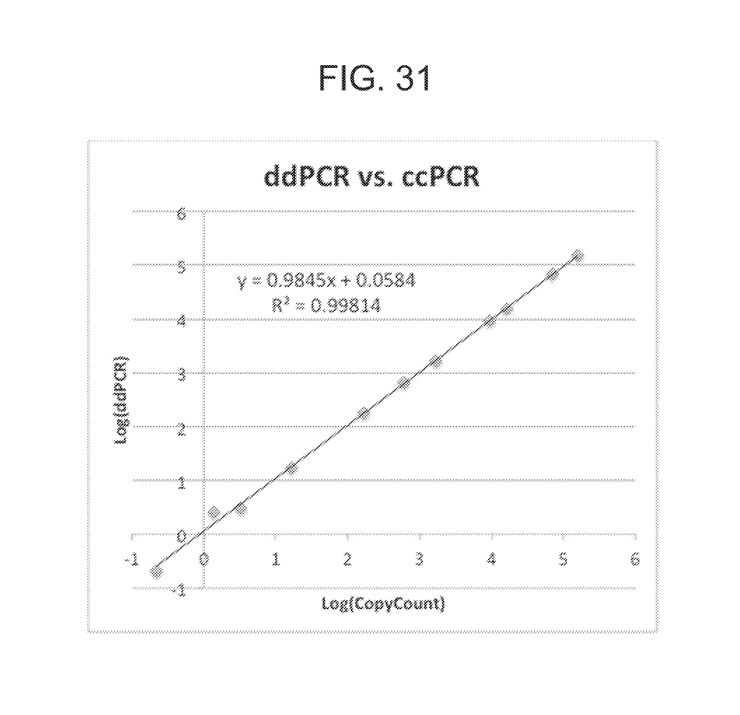

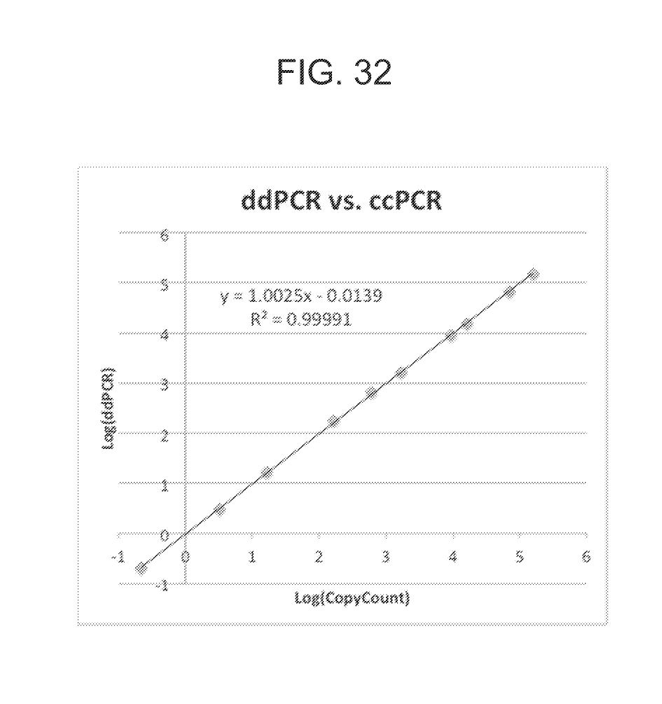

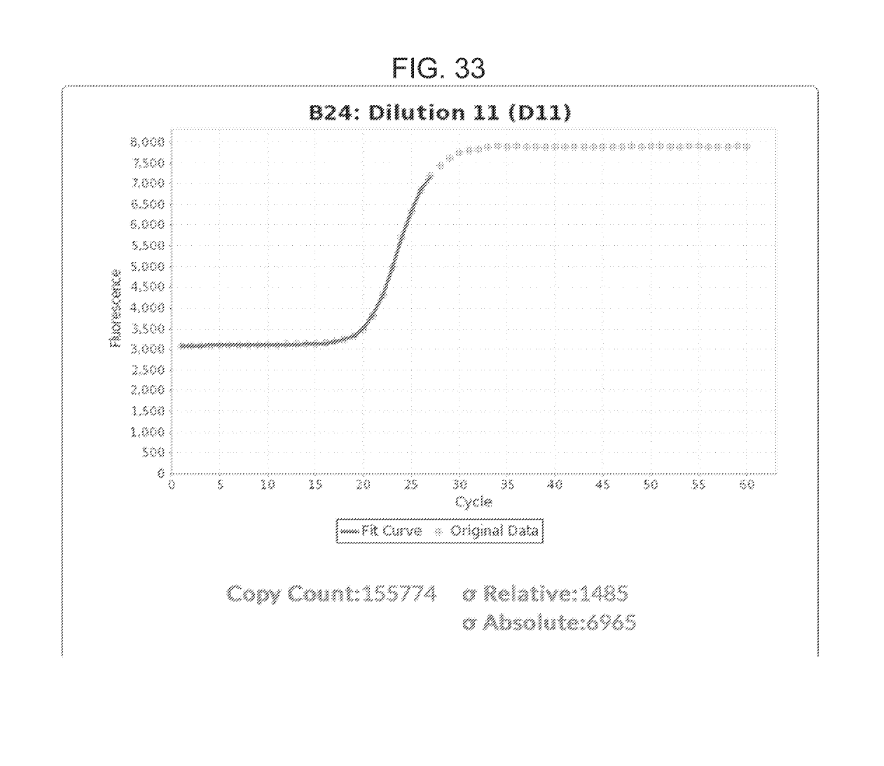

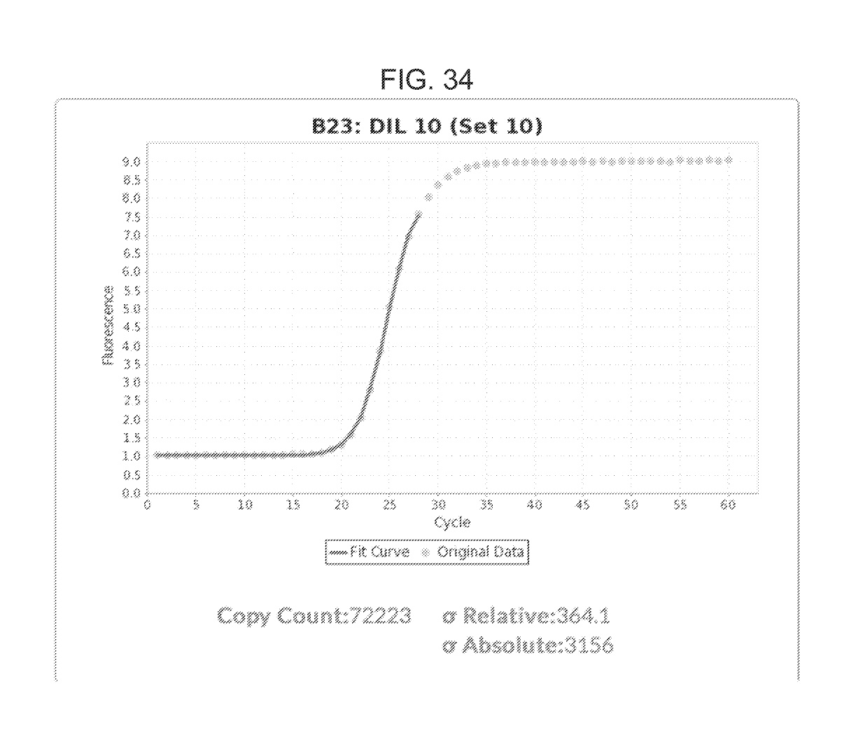

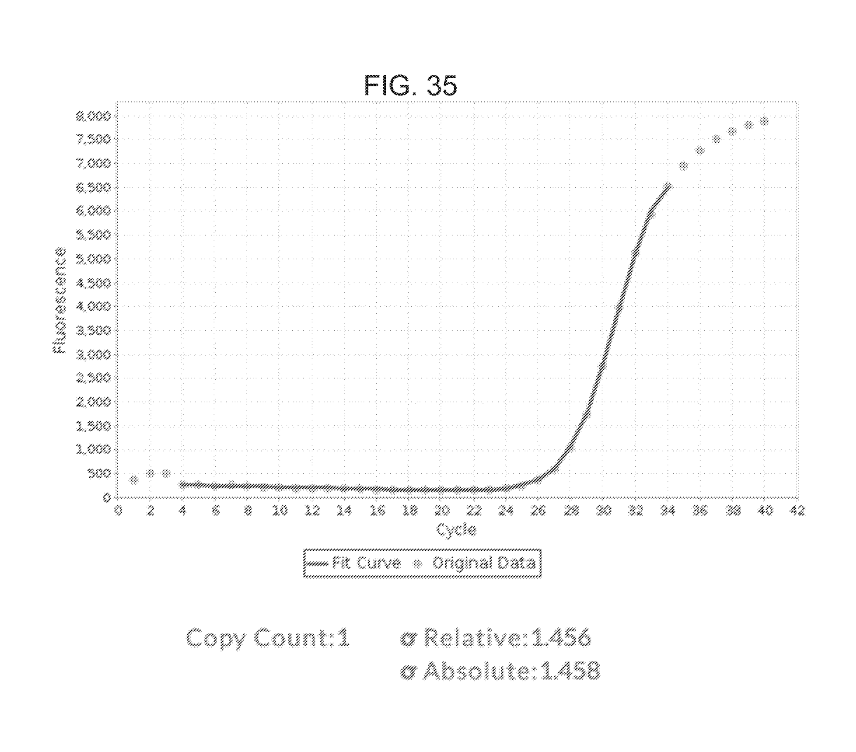

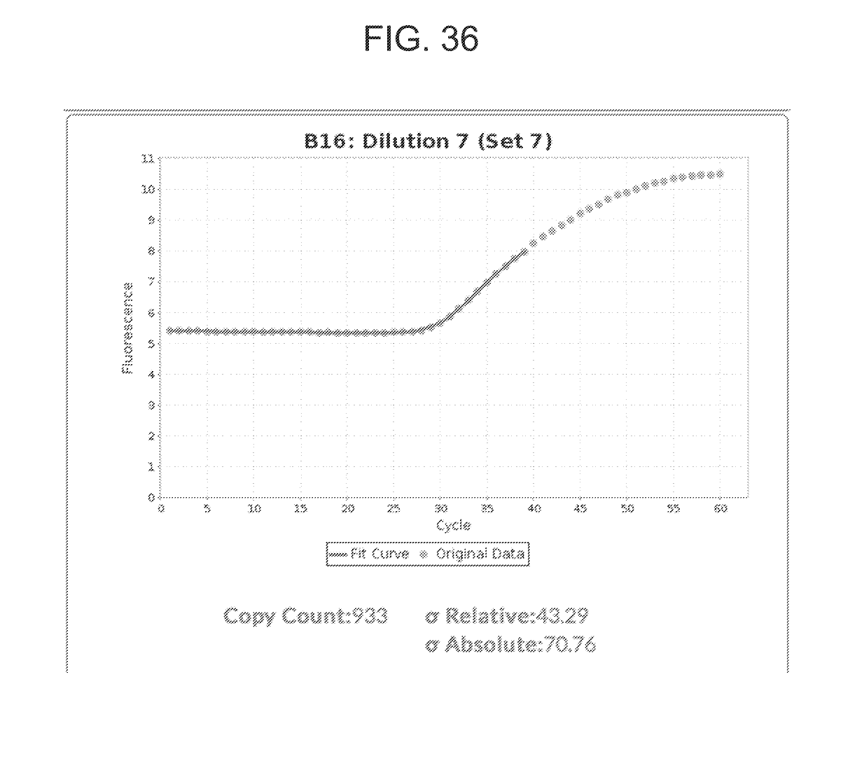

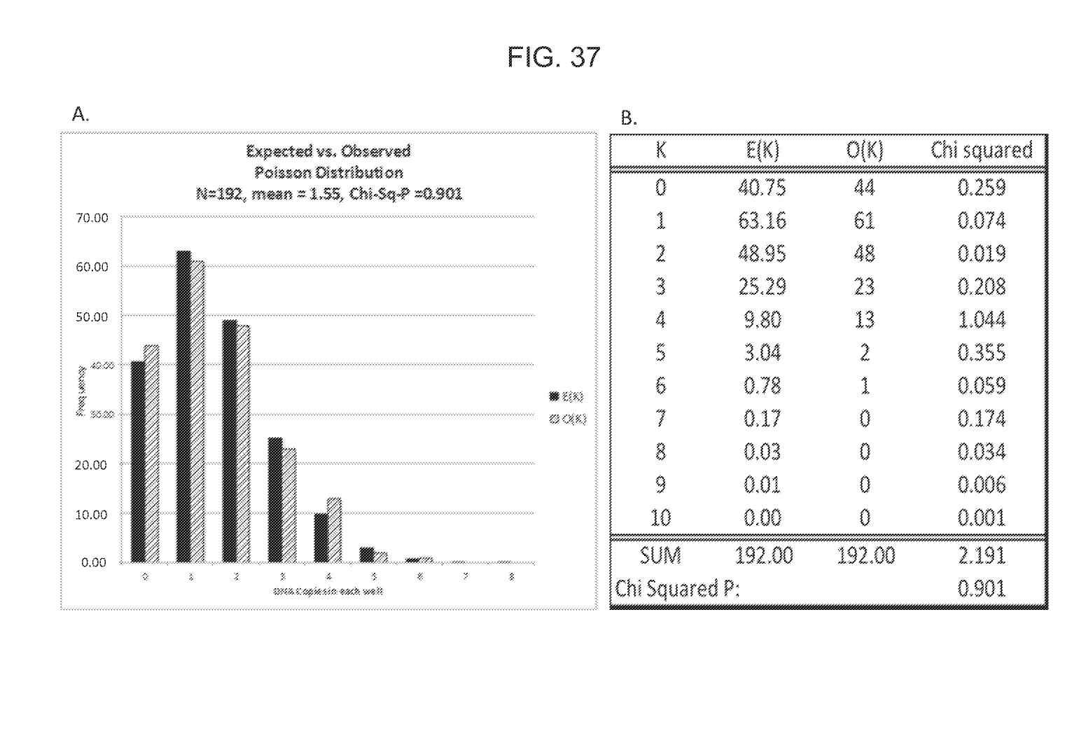

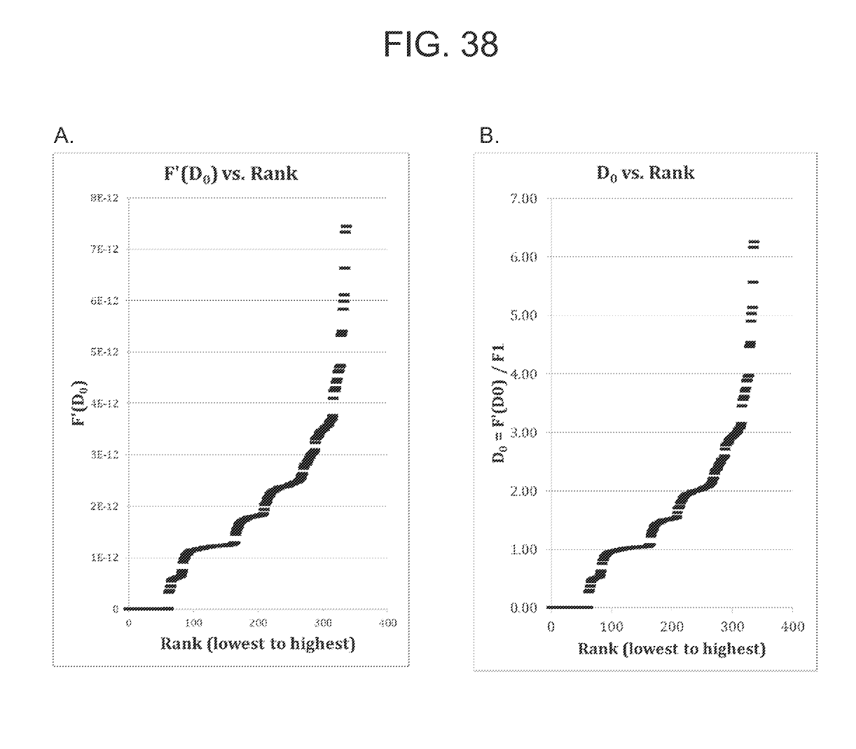

To illustrate the fundamental difference between accuracy and precision, the analogy of shooting a target is instructive (FIG. 29). The far left panel of FIG. 29 shows the case of a rifle with calibrated sighting scope in the hands of a professional marksman with a steady hand. The middle left panel is the result for a professional marksman using a rifle whose sighting scope is not calibrated. The middle right panel is the result for an amateur (with a shaky hand) using a calibrated rifle. The far right panel is for an amateur shooting an un-calibrated rifle.

Applying this analogy to qPCR, each shot of the rifle corresponds to a single qPCR reaction. The σ_{Relative} is a measure of precision or random error. σ_{Calibration} is a measure of the systematic error or accuracy. The σ_{Absolute} is the total error that results from both σ_{Relative} and σ_{Calibration}. Factors that contribute to σ_{Relative} include the Poisson sampling error (described below), pipetting errors in the amounts of target and other reagents, and noise in the qPCR data. Averaging the replicates reduces the random error and results in a smaller σ_{Relative}, which represents the standard error of the mean for the replicate set. The more replicates that are performed, the smaller the σ_{Relative}. The σ_{Calibration} can be improved by performing a calibration plate with more replicates and using the proper mean copy number.

Good Practices for Copy Counting Using qPCR:
1. For unknown samples, perform as many replicates as possible to decrease the relative error.
2. Calibrate pipettes and use good pipetting technique to reduce the random and systematic errors (thereby improving both relative and absolute quantification).
3. Perform a calibration plate measurement using the maximum number of replicates permitted by the plate size. The calibration plate generally needs to be performed only once for each assay design, and is instrument- and sample-independent. Performing a large number of replicates for the calibration plate gives the subsequent qPCR copy counting runs for that assay design the highest possible accuracy.
   Note: If no calibration plate is run, then the copy count results are nonetheless highly precise and thus reliable for relative quantification, but the absolute quantification inaccuracy may be within 20-30%, or higher.

### Basic Error Analysis:

About replicates. Performing replicate PCR experiments allows the calculation of the standard deviation among those replicates:

$${σ}_{Replicate} = \sqrt{\frac{1}{N - 1} {\sum }_{i = 1}^{N} {( {CC}_{i} - μ )}^{2}} \qquad ( Eqn . 73 )$$

where N is the number of replicates, μ is the average copy count of the replicates, and CCᵢ is the copy count number for well i. The σ_{Replicate} represents the expected error for a single qPCR well. The Poisson sampling error is given by the square root of the copy count.

Thus, if the mean copy count is small (less than 400 copies), then the Poisson sampling error is the dominant contribution to the σ_{Replicate}. Such Poisson error occurs even if the user pipettes perfectly.

The average, or mean, is significantly more reliable than a single measurement. The standard error in the mean, σ_{Mean} (also called σ_{Relative}), is given by:

$${σ}_{Mean} = {σ}_{Relative} = \frac{{σ}_{Replicate}}{\sqrt{N}} \qquad ( Eqn . 74 )$$

Thus, performing more replicates can dramatically reduce the error in the mean (i.e. σ_{Relative}). For example, performing 16 replicates results in 4-fold smaller error than a single qPCR reaction.

The coefficient of variation, CV, is the ratio of the error divided by the mean. Thus, the CV_{Relative} is given by:

$${CV}_{Relative} = \frac{{σ}_{Relative}}{μ} \qquad ( Eqn . 75 )$$

The absolute error is the combination of the relative error and the calibration error as follows:

CV_{Absolute}+\sqrt{CV_{Calibration}²+CV_{Relative}²} (Eqn. 76)

The table below illustrates the effects of different combinations of calibration and relative errors. Note the absolute errors are dramatically smaller for calibrated assays than for uncalibrated assays.

TABLE 1
Effect of different Calibration and Relative errors on the Absolute
Error from Equation 4.

| Case | CV_{Calibration} | CV_{Relative} | CV_{Absolute} |
|---|---|---|---|
| Accurate and Presise | 0.03 | 0.01 | 0.032 |
| Inaccurate and Precise | 0.20 | 0.01 | 0.200 |
| Accurate and Imprecise | 0.03 | 0.04 | 0.050 |
| Inaccurate and Imprecise | 0.20 | 0.04 | 0.204 |

Notes:
Errors shown are typical for if the calibration plate has 384 wells (i.e. CV_{Calibration} = 3%) and 16 replicates (CV_{Relative} = 1%) are performed on the unknowns. Assays that are uncalibrated are assumed to have 20% calibration error. A sample with only a single replicate (imprecise) are assumed to have 4% relative error.

### Example 3

### Two-Step Calibration Procedure for Determining U

In some embodiments, the following procedure is performed on each new qPCR assay that will be analyzed by the qPCR copy counting method. The method is called “2-step” because it involves two qPCR reactions: one preliminary PCR with 4 replicates to get a rough concentration measurement, and one full plate of PCR reactions to get the precise calibration. This method is faster, more accurate, and more reliable than a dilution series with standards.

The calibration needs to be performed only once on each new assay design, i.e. the same calibration will work on any instrument and with any sample and will never need to be redone as long as the primers are not redesigned, the primer and probe concentrations are not changed, and the PCR buffer components (i.e. [NTPs], [Mg], [Enzyme], and enzyme type) are not changed. Thus, it is generally best to perform the calibration once with as many replicates as possible so that the assay can be used in the future with optimal accuracy.

As discussed above, the calibration error, σ_{calibration} depends upon the number of replicates and the mean copy number among the replicates. A 384-well calibration of an assay will provide σ_{calibration} of about 5% inaccuracy if the mean copy number per well is 1.5. For a 96 well plate, the calibration errors will be twice as large as from a 384 well plate. Below is the equation for calculating the approximate calibration error (for M<1.5):

$$Calibration\_Error = \frac{1}{\sqrt{N \times M}} \qquad ( Eqn . 77 )$$

where N is the number of replicates and M is the average copy number per well. Note that for technical reasons, it is not advisable to go above a copy number of 3.5 in performing your calibration plate. Thus, to give a little safety margin, we recommend that you use a mean copy number of about 1.5 for the calibration plate. We also strongly recommended that you use a calibration plate with as many replicates as possible so that error is minimized.

Example of a Laboratory Protocol for Running a Calibration Plate:

Note: If you know your initial DNA concentration very accurately (within 25% error), then you can skip step 1 and go directly to step 2.

Step 1A. Initial PCR. This protocol is written assuming 20 μL qPCR reactions. If your instrument uses a different volume, then scale the amounts of target and other reagents such as master mix and primers and probes accordingly. Prepare a 10 μL sample, labeled “Target DNA”, that contains between 1,000 and 100,000,000 copies of Target DNA (no need to be wasteful here, we just need at least 1000 molecules for the entire calibration procedure). Add 2 μL of the target DNA to a centrifuge tube labeled “reaction mix”. Add to the “reaction mix” tube 50 μL of 2× master mix (or 10 μL of 10× master mix) and appropriate volumes of primers and probe. Add water to make the final volume=100 μL, which is sufficient for 5 PCR reactions, but only 4 PCR reactions will be run. Mix well and pipette 20 μL of the resulting mix into each of four reaction wells in the qPCR plate. The excess ˜20 μL can be discarded (100 μL of reaction mix was prepared to be sure that there is enough for the 4 reactions to get a full 20 μL).

Step 1B. Obtain Estimated Copy Count. Run qPCR CopyCount on the four qPCR reactions from step 1A. This will give a rough estimate of the copy count, CC. Average the CC for the four replicates. This estimate generally provides the DNA copy number to within ±30%.

Notes:
1. It is important to acquire a sufficient number of PCR cycles to allow for saturation to be observed. For example, 55 cycles for 10-20 μL reaction volumes (if the volume is much smaller, e.g., 33 nL, then fewer cycles can be used as long as full saturation is observed even for a single copy of DNA at cycle zero).
2. PCR extension time of 1 minute is recommended to ensure that all amplicons are fully extended.

Step 2A. Prepare Calibration Plate. The goal of this step is to prepare a PCR reaction sufficient for 400 wells that each contain about 1.5 molecules of DNA on average (so a total or 600 target molecules are needed). Compute the total molecules that remain in the 8 uL Target DNA sample from step 1A. This is accomplished using the copy count, CC, from step 1B as follows:

Total Target DNA Molecules−CC×20 (Eqn. 78)

where the factor of 20 is because the remaining Target sample has 4-fold as much DNA in 8 μL compared to 2 μL, and that was effectively split into 5 reactions worth of volume in step 1A. From the total from Eqn. 2, compute the volume that contains 600 molecules. For example, if the Total=1352 molecules then the volume needed is:

$$Volume Needed = 8 µL \times \frac{600 molecules}{1352 Total molecules} = 3.6 µL \qquad ( Eqn . 79 )$$

Note that the volume used does not need to be perfectly exact (for example if you pipetted 3.5 μL that would be fine), the number of molecules could be off by a few percent and that will have no effect on the calibration. If the volume computed with Eqn. 3 is too small (like 0.01 μL), then you will need to first dilute the sample by adding water, and then pipetting out the amount needed taking into account the added dilution. Pipette the volume needed from Eqn. 3 into a fresh 20 mL tube labeled “Calibration Reaction Mix”. Since we are preparing reaction mixture for 400 reactions with 20 μL each, the total reaction volume is 8000 μL. Add to the “calibration reaction mix” tube 800 μL of 10× qPCR components (master mix, primers and probe) and add water to make the final volume=8000 μL, which is sufficient for 400 PCR reactions, but only 384 PCR reactions will be run.

Step 2B. Run Your Calibration Plate. Run qPCR CopyCount and select “Calibration Plate”. Upload the data from step 2A, and give a name for the assay that you are calibrating. The program will do the rest. The calibration for that assay will be saved to your database of assays so that you can use it for sample unknowns in the future.

Notes:
1) Rarely, some assays may be very poorly designed resulting in aberrant behavior. If your calibration produces a message “Calibration plate unreliable due to poor Chi-squared P”, this is an indication that your assay is poorly designed (e.g. the primers are highly inefficient due to competing secondary structure) or that there is some other problem with the PCR, such as very bad contamination or poor reagent quality.
2) If your estimated copy count in step 1B is above 3.5, then you will get the message: “Calibration plate unreliable due to high copy number”. This means that you will need to further dilute your sample and run a new calibration plate.

### Example 4

### Calculation of Copy Counting Calibration Error and Poisson Error

This pseudocode computes the error in the CopyCount calibration error and the Poisson error.

```
//Calculation of CopyCount Calibration Error
double mean = 1.0; //this is the actual mean set by the user
int N=96; //this is the number of wells in the calibration plate
double sum=0;
double CC_mean[1000]=0; //single dimension array that stores trial CopyCount means
double Poisson_mean[1000] =0; single dimension array that stores trial Poisson means
int zeros_count = 0;
For(j=0;j<=999;j++) //perform 1000 trials
{
      For(i=1;i<=N;i++)
      {
            int X = rand_poisson(mean) //generates a random integer from a Poisson
distribution with given mean
            sum += double(X);
            if(X==0) zeros_count += 1;
      }
      CC_mean[j]= sum/double(N); //this is the trial CopyCount mean
      if(zeros_count != 0 && zeros count !=N)
            Poisson_mean[j] = −LN(1−(N− zeros_count)/N);
      else j−−; //decrement j to repeat any trial that has no zeros or all zeros
}
Calibration_error = Stdev (CC_mean[j] ) / mean; //compute standard deviation of the 1000
trial CopyCount means and divide by the mean to get the calibration error.
Poisson_error = Stdev (Poisson_mean[j]) / mean; //compute standard deviation of the 1000
Poisson means and divide by the mean to get the Poisson error.
```

### Example 5

### Initial Estimation of Parameters for Probe Deletion Model

Inputs From the User:
Volume
[primer]
[probe]
DS=true/false

List of Unknowns:
F_{b}, M, D₀, pdK, PDC, CL, F₁

Procedure for Estimating Initial Guesses for All Unknowns:
1. Determine if the data set has at least 5 points above the inflection point. If the number of points above inflection is greater than 4, then use steps 2-11 below. Otherwise, fit the well using the primer depletion model.
2. Assume that Volume, [primer], and [probe] are all fixed values (i.e. don't let them float).
3. Get F_{b} and M from preliminary MAK2 fitting.
4. Use the log-logistic to get the inflection point. We will need both the X and Y values of the inflection point. The Y-value needs to be background corrected. We will call the X-value of the inflection of the log-logistic=X_{inflection}^{log-log}.
5. Initially assume CL=1. CL is only allowed to vary between 0 and 1.
6. Initially assume pdK=0.25. pdK must be >0 (i.e. it is not allowed to be negative or zero).
7. Initially assume PDC=2E-8. PDC can vary between 0 to infinity. Note that in the limit where PDC=0 is simply the limit where all targets are bound by probes, which uses up the probes in an abrupt manner.
8. Computation of F₁ is as follows:
   if there are 10 points above the inflection point then estimate F₁:

$${F}_{1} = \frac{F ( IP + 10 ) - F ( IP - 10 )}{{B}_{0} \times V \times {N}_{A}} \qquad ( Eqn . 12 )$$

else estimate F₁ differently:

$${F}_{1} = \frac{2 \times ( F ( IP ) - F ( IP - 10 ) )}{{B}_{0} \times V \times {N}_{A}} \qquad ( Eqn . 12 B )$$

where IP is the x-value of the inflection point from the log-logistic fitting from step 3. F(IP+10) means the background corrected fluorescence at the cycle inflection x-value+10. If there are fewer than 10 points below the inflection point, then use the of the first point for F(IP-10).
9. Lastly, we estimate the D₀ value. First we assume D₀=1 and then simulate the PCR curve using all the other estimated parameters given in steps 1-6 and using all the equations 2-11. For that simulated curve with D₀=1, fit the log-logistic to that to get its inflection point, which we will call X_{inflection}^{D0=1}. We then compute an estimate of D₀ as follows:

$${D}_{0} = {2}^{{X}_{inflection}^{D 0 = 1} - {X}_{inflection}^{log - log}} \qquad ( Eqn . 13 )$$

if the resulting D₀<1, then set D₀=1.
10. Fit the curve using Marquart non-linear least squares method, allowing F_{b}, M, D₀, pdK, PDC, CL, and F₁ to float (i.e. 7 unknowns). P₀ and B₀ are fixed to the values given by the user.
11. In some embodiments it is useful to perform the fitting in stages in which some parameters are allowed to vary (V), while others are fixed (F). The table below presents one such strategy.

| Parameter | Round 1 | Round 2 | Round 3 |
|---|---|---|---|
| D₀ | V | V | V |
| P₀ | F | F | F |
| B₀ | F | F | F |
| F_{b} | V | F | F |
| M | V | F | F |
| pdK | V | V | V |
| PDC | F | V | V |
| CL | F | F | V |
| F1 | V | V | V |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1. A method for determining the amount of a target nucleic acid, comprising:
a) providing a quantitative amplification data set from a target nucleic acid in a sample,
i) wherein providing said quantitative amplification data set comprises exposing said one or more samples to amplification reaction reagents for quantitative amplification under conditions wherein said target nucleic acid is amplified, and wherein quantitative amplification data is collected,
ii) wherein said quantitative amplification data set describes a curve having an intrinsic reference point F_{reference}, wherein F_{reference} is selected from the group consisting of Fₘₐₓ_{_}_{curvature}, and Fₘₐₓ_{_}_{second}_{_}_{derivative}, F_{inflection}, Fₘᵢₙ_{_}_{second}_{_}_{derivative}, Fₘᵢₙ_{_}_{curvature}, and Fₘᵢₙ, wherein Fₘₐₓ_{_}_{curvature} is the fluorescence at a point of maximum curvature of said curve, Fₘₐₓ_{_}_{second}_{_}_{derivative} is the fluorescence at the point of the maximum of the second derivative F_{inflection} is the fluorescence at the inflection point in a curve of said quantitative amplification data set, Fₘᵢₙ_{_}_{second}_{_}_{derivative} is the fluorescence at the point of the minimum of the second derivative, Fₘᵢₙ_{_}_{curvature} is the fluorescence at a point of minimum curvature of said curve, and Fₘₐₓ is the fluorescence at saturation of curve;
b) providing a mass action kinetic model of exponential amplification comprising parameters F(D₀), pdK, Pₙ, F_{b}, and Fₘ, wherein F(D₀) represents the amount of signal from double-stranded DNA at cycle 0, which is proportional to D₀, the amount of double stranded DNA at cycle 0; pdK is a constant related to the rate constant for primer hybridization divided by the rate constant determining the rate of DNA amplicon reannealing during a quantitative amplification reaction, Pₙ is the concentration of primer for cycle n; F_{b} represents a constant background fluorescence present in the data; and Fₘ represents the slope of the background fluorescence present in the data, and optionally comprising parameters Bₙ and/or EC, wherein Bₙ, is the concentration of probe at cycle n and, EC for efficiency of probe cleavage, wherein said mass action kinetic model of quantitative amplification comprises the relation:
$${D}_{n} = {D}_{n - 1} + k ln ( 1 + \frac{{D}_{n - 1}}{k} ) ,$$
wherein Dₙ represents an amount of DNA in a sample at the end of an nth cycle of a quantitative amplification reaction, and k is a constant;
c) fitting said quantitative data set with said mass action kinetic model of a quantitative amplification reaction to determine a value F(D₀) for said target nucleic acid; and
e) determining a value for F′(D₀), wherein said value F′(D₀) is proportional to D₀, the amount of target nucleic acid in said sample, wherein determining the value for F′(D₀) comprises determining a value for signal at an intrinsic reference point F_{reference} in said quantitative amplification data set.

2. (canceled)

3. The method of claim 1, wherein said quantitative amplification data set comprises data from an amplification reaction comprising a forward primer and a reverse primer, wherein the initial concentration of the forward primer, FP₀, is or is not the same as the initial concentration of the reverse primer, RP₀, and wherein said mass action kinetic model of exponential amplification comprises the parameters FP_{N} and RP_{N}, wherein FP_{N} is the concentration of forward primer for cycle n, and RP_{N} is the concentration of reverse primer for cycle n, wherein FP_{N} at cycle N is determined according to the equation:
FP_{N}=FP_{N-1}−(LS_{N}−LS_{N-1})−(AS_{N}−AS_{N-1})
and wherein RP_{N} at cycle N is calculated according to the equation:
RP_{N}=RP_{N-1}−(LA_{N}−LA_{N-1})−(AA_{N}−AA_{N-1}).

4. (canceled)

5. The method of claim 2, wherein the value for F′(D₀) is determined from the value of F(D₀) according to the equation:
$${F}^{′} ( {D}_{0} ) = \frac{F ( {D}_{0} )}{{F}_{reference}} .$$

6. (canceled)

7. The method of claim 1, wherein F_{reference} is F_{inflection}.

8. The method of claim 1, further comprising a step of determining a value for D₀, wherein D₀ is proportional to the value F′(D₀) according to Equation 40:
D₀=U×F′(D₀) (40)
wherein U is a universal constant, wherein a value for U is determined using a process comprising:
i) providing a target nucleic acid in a quantitative amplification reaction mixture of volume V, wherein a single copy of said target nucleic acid is present in said mixture;
ii) exposing said mixture to conditions under which said target nucleic acid is amplified to produce a quantitative amplification data set,
iii) determining values for F(D₀=1 copy) and F_{reference} from said quantitative amplification dataset;
iv) calculating a value for F′(D₀=1 copy), wherein
$${F}^{′} ( {D}_{0} ) = \frac{F ( {D}_{0} )}{{F}_{reference}}$$
and
v) calculating U, wherein
$${D}_{reference} = U \times V = \frac{1}{{F}^{′} ( {D}_{1 copy} )} \qquad ( 32 )$$
and
vi) optionally, computing a fraction of delayed onset,
wherein a DNA copy number for said target nucleic acid at cycle 0 is calculated according to Equation 33:
D₀=U×V×F′(D₀) (33)

9. (canceled)

10. The method of claim 1, wherein said quantitative amplification data set is a truncated quantitative amplification data set provided by exposing said one or more samples to amplification reaction reagents under conditions wherein said target nucleic acid is amplified and wherein quantitative amplification data is collected, wherein collection of quantitative amplification data is terminated at a threshold fluorescence value to generate said truncated quantitative amplification data sets.

11. (canceled)

12. The method of claim 1, wherein said quantitative amplification data set from said target nucleic acid comprise a plurality of replicate quantitative amplification data sets for target nucleic acid, wherein a mean fitting error component sigma_mean_F′(D₀) is determined from the replicate quantitative amplification data set for said target nucleic acid.

13.-17. (canceled)

18. The method of claim 1, wherein fluorescence from DNA at cycle n F′(Dₙ) is calculated from fluorescence observed during the nth cycle of said polymerase chain reaction (Fₙ), and wherein Fₙ=F′(Dₙ)+Fₘ*n+F_{b}.

19.-20. (canceled)

21. The method of claim 1, wherein said providing the quantitative amplification data set comprises providing a plurality of replicate mixtures, each comprising a zero, one or a plurality of copies of said target nucleic acid in a quantitative amplification reaction mixture, wherein said exposing comprises exposing said plurality of replicate mixture of volume V to said conditions such that a plurality of replicate quantitative amplification data sets is produced; and wherein said determining values for F(D₀=1 copy) comprises averaging one or more data points from said plurality of replicate quantitative amplification data sets, and F_{reference} is one of the six intrinsic reference points.

22. A system for absolute quantitation of target nucleic acid in a sample, the system comprising:
a) a real-time PCR apparatus for acquiring quantitative amplification data sets, wherein a quantitative amplification data set describes a curve having an intrinsic reference point F_{reference}, and wherein F_{reference} is selected from the group consisting of Fₘₐₓ_{_}_{curvature}, and Fₘₐₓ_{_}_{second}_{_}_{derivative}, F_{inflection}, Fₘᵢₙ_{_}_{second}_{_}_{derivative}, Fₘᵢₙ_{_}_{curvature}, and Fₘₐₓ, wherein Fₘₐₓ_{_}_{curvature} is the fluorescence at a point of maximum curvature of said curve, Fₘₐₓ_{_}_{second}_{_}_{derivative} is the fluorescence at the point of the maximum of the second derivative, F_{inflection} is the fluorescence at the inflection point in a curve of said quantitative amplification data set, Fₘᵢₙ_{_}_{second}_{_}_{derivative} is the fluorescence at the point of the minimum of the second derivative, Fₘᵢₙ_{_}_{curvature} is the fluorescence at a point of minimum curvature of said curve, and Fₘₐₓ is the fluorescence at saturation of curve;
b) a microprocessor configured to:
i) fit quantitative amplification data sets with a mass action kinetic model of exponential amplification comprising parameters F(D₀), pdK, Pₙ, F_{b}, and Fₘ, wherein F(D₀) represents the amount of signal from single or double-stranded DNA at cycle 0, which is proportional to D₀, the amount of double stranded DNA at cycle 0; pdK is a constant related to the rate constant for primer hybridization divided by the rate constant for amplicon reannealing of sense and antisense strands; Pₙ is the concentration of primer for cycle n; F_{b} represents a constant background fluorescence present in the data; and Fₘ represents the slope of the background fluorescence present in the data, and optionally comprising parameters Bₙ, PDC, and/or EC, wherein Bₙ, is the concentration of probe at cycle n, PDC is the apparent probe dissociation constant, and, EC is the efficiency of probe cleavage; and
ii) calculate a value for F′(D₀), wherein said value F′(D₀) is proportional to D₀, the amount of target nucleic acid in said sample.

23. The system of claim 22, wherein said quantitative amplification data sets comprise data from amplification reactions comprising a forward primer and a reverse primer, wherein the initial concentration of the forward primer, FP₀, is or is not the same as the initial concentration of the reverse primer, RP₀, and wherein said mass action kinetic model of exponential amplification comprises the parameters FP_{N} and RP_{N}, wherein FP_{N} is the concentration of forward primer for cycle n, and RP_{N} is is the concentration of reverse primer for cycle n, wherein FP_{N} at cycle N is calculated according to the equation:
FP_{N}=FP_{N-1}−(LS_{N}−LS_{N-1})−(AS_{N}−AS_{N-1})
and wherein
RP_{N} at cycle N is calculated according to the equation:
RP_{N}=RP_{N-1}−(LA_{N}−LA_{N-1})−(AA_{N}−AA_{N-1}).

24. (canceled)

25. The system of claim 22, wherein said microprocessor is further configured to determine a value for signal at an intrinsic reference point F_{reference} in said quantitative amplification data set, and wherein the value for F′(D₀) is determined from the value of F(D₀) according to the equation:
$${F}^{′} ( {D}_{0} ) = \frac{F ( {D}_{0} )}{{F}_{reference}}$$

26. (canceled)

27. The system of claim 25, wherein said microprocessor is further configured to calculate a value for D₀, wherein D₀ is proportional to the value F′(D₀) according to Equation 40:
D₀=U·F′(D₀) (40)

28. (canceled)

29. The system of claim 22, further comprising functionality to output a result reporting a value for D₀ or a value derived from the value for D₀, wherein said value derived from the value for D₀ comprises a concentration of target nucleic acid in a volume, or a value for a number of target nucleic acid copies in a sample.

30. (canceled)

31. The system of claim 29, wherein the result is understandable by a human.

32. A method for determining the relative amounts of a plurality of target nucleic acids, comprising:
a) providing quantitative amplification data sets from at least two target nucleic acids in one or more samples;
b) providing a mass action kinetic model of exponential amplification comprising parameters F(D₀), pdK, Pₙ, F_{b}, and Fₘ, wherein F(D₀) represents the amount of signal from double-stranded DNA at cycle 0, which is proportional to D₀, the amount of double stranded DNA at cycle 0; pdK is a constant related to the rate constant for primer hybridization divided by the rate constant determining the rate of DNA amplicon reannealing during a quantitative amplification reaction; Pₙ is the concentration of primer for cycle n; F_{b} represents a constant background fluorescence present in the data; and Fₘ represents the slope of the background fluorescence present in the data and optionally comprising parameters Bₙ and/or EC, wherein Bₙ, is the concentration of probe at cycle n and, EC for efficiency of probe cleavage;
c) fitting each of said quantitative data sets with said mass action kinetic model of a quantitative amplification reaction to determine a value F(D₀^{A}) for a target nucleic acid A, and a value F(D₀^{B}) for a target nucleic acid B;
d) calculating from F(D₀^{A}) and F(D₀^{B}) an intrinsically referenced value F′(D₀^{A}) for a target nucleic acid A, and an intrinsically referenced value F′(D₀^{B}) for a target nucleic acid B, wherein said value F′(D₀^{A}) is proportional to D₀^{A}, the amount of target nucleic acid A, and said value F′(D₀^{B}) is proportional to D₀^{B}, the amount of target nucleic acid B in said one or more samples;
d) determining a ratio D₀^{A}/D₀^{B} to determine the relative amounts of target nucleic acid A and target nucleic acid B in said one or more samples.

33. The method of claim 32, wherein said quantitative amplification data sets comprise data from amplification reactions each comprising a forward primer and a reverse primer, wherein the initial concentration of the forward primer, FP₀, is or is not the same as the initial concentration of the reverse primer, RP₀, and wherein said mass action kinetic model of exponential amplification comprises the parameters FP_{N} and RP_{N}, wherein FP_{N} is the concentration of forward primer for cycle n, and RP_{N} is is the concentration of reverse primer for cycle n, wherein FP_{N} at cycle N is calculated according to the equation:
FP_{N}=FP_{N-1}−(LS_{N}−LS_{N-1})−(AS_{N}−AS_{N-1})
and wherein RP_{N} at cycle N is calculated according to the equation:
RP_{N}=RP_{N-1}−(LA_{N}−LA_{N-1})−(AA_{N}−AA_{N-1})

34. (canceled)

35. The method of claim 32, wherein the ratio D₀^{A}/D₀^{B} is determined according to the Equation 39:
$$\frac{{D}_{o}^{A}}{{D}_{o}^{B}} = \frac{U \cdot {F}^{′} ( {D}_{o}^{A} )}{U \cdot {F}^{′} ( {D}_{o}^{B} )} = \frac{{F}^{′} ( {D}_{o}^{A} )}{{F}^{′} ( {D}_{o}^{B} )} \qquad ( 39 )$$

35. (canceled)

36. The method of claim 32, wherein target nucleic acid A and target nucleic acid B have different nucleic acid sequences.

37. (canceled)

38. The method of claim 32, wherein target nucleic acid A and target nucleic acid B are from different samples.
